(19) United States
(12) Patent Application Publication
(10) Pub. No.: US 2019/0352373 A1
(43) Pub. Date: Nov. 21, 2019

(54) TGF-ß DECOY RECEPTOR

(71) Applicant: Tessa Therapeutics Pte. Ltd., Singapore (SG)

(72) Inventors: John Edward Connolly, Singapore (SG); Richard Hopkins, Singapore (SG); Yijie Philbert Ong, Singapore (SG); Sven Hans Petersen, Singapore (SG)

(73) Assignee: Tessa Therapeutics Pte. Ltd., Singapore (SG)

(21) Appl. No.: 16/480,391

(22) PCT Filed: Jan. 19, 2018
(86) PCT No.: PCT/EP2018/051287

§ 371 Date: Jul. 24, 2019

Related U.S. Application Data
(60) Provisional application No. 62/450,468, filed on Jan. 25, 2017.

(51) Int. Cl. C07K 14/71; A61P 35/00; A61K 35/17; C12N 15/85; A61K 47/54; C07K 14/725

(52) U.S. Cl. CPC C07K 14/71; A61P 35/00; C07K 14/7051; C12N 15/85; A61K 47/544; A61K 35/17

(57) ABSTRACT

A TGF-β decoy receptor comprising a TGF-β binding region and a lipid anchor region is disclosed. Also disclosed is a chimeric antigen receptor (CAR) comprising a TGF-β binding region. Also disclosed are compositions comprising, and methods using, the TGF-β decoy receptors and CARs.

### FIELD OF THE INVENTION

The present invention relates to decoy receptors of transforming growth factor β (TGF-β).

BACKGROUND TO THE INVENTION

Immunotherapy with ex vivo expanded or genetically modified T cells has shown great promise in the treatment of hematologic malignancies. However, the long term persistence of transferred T cells is limited by their activation state and interaction with host associated factors. Additionally, overcoming the suppressive nature of the tumor microenvironment remains a major challenge for T cell immunotherapy.

Transforming growth factor β (TGF-β) is an immunosuppressive cytokine whose expression is upregulated in a variety of cancers. TGF-β signals through the heterodimeric TGF-β receptor complex, which is made up of type-1 and type-2 subunits. Signalling through the TGF-β receptor has been shown to inhibit T cell proliferation, in vivo persistence, and effector function.

### SUMMARY OF THE INVENTION

In one aspect, the present invention provides a TGF-β decoy receptor comprising, or consisting of, a TGF-β binding region and a cell membrane anchor region. In some embodiments, the cell membrane anchor region comprises or consists of a lipid anchor. In some embodiments, the lipid anchor is a GPI anchor.

In another aspect, the present invention provides a TGF-β decoy receptor comprising, or consisting of, a TGF-β binding region and a lipid anchor region.

The TGF-β decoy receptor may be a fusion protein comprising or consisting of, the extracellular domain of a TGF-β receptor covalently linked to a cell membrane anchor region, optionally via a linker.

In some embodiments, the lipid anchor region comprises or consists of a lipid anchor. In some embodiments, the lipid anchor is a GPI anchor.

In some embodiments, the TGF-β decoy receptor lacks a transmembrane domain.

In some embodiments, the lipid anchor region comprises or consists of an amino acid sequence which is a lipid anchor signal sequence. In some embodiments, the lipid anchor signal sequence is a glycosylphosphatidylinositol (GPI) signal sequence.

In some embodiments, the TGF-β binding region comprises or consists of an amino acid sequence corresponding to the TGF-β binding region of a TGF-β receptor. In some embodiments, the TGF-β receptor is the type II TGF-β receptor (TGF-βR2).

In some embodiments, the TGF-β binding region comprises or consists of an amino acid sequence having at least 80% amino acid sequence identity to SEQ ID NO:4.

In some embodiments, the cell membrane anchor region comprises or consists of an amino acid sequence corresponding to the GPI signal sequence of one of CD48, CD44, CD55, CD90 or placental-type alkaline phosphatase.

In some embodiments, the lipid anchor region comprises or consists of: (i) an amino acid sequence having at least 80% amino acid sequence identity to any one of SEQ ID NOs:15 to 18; or (ii) a GPI anchor, and an amino acid sequence having at least 80% amino acid sequence identity to any one of SEQ ID NOs:19 to 22.

In some embodiments, the TGF-β decoy receptor comprises or consists of: (i) an amino acid sequence having at least 80% amino acid sequence identity to any one of SEQ ID NOs:5 to 8; or (ii) an amino acid sequence having at least 80% sequence identity to one of SEQ ID NOs:11 to 14, and a GPI anchor.

In another aspect, the present invention provides a chimeric antigen receptor (CAR) comprising a TGF-β binding region which comprises or consists of an amino acid sequence corresponding to the TGF-β binding region of a TGF-β receptor.

In some embodiments, the TGF-β receptor is the type II TGF-β receptor (TGF-βR2). In some embodiments, the TGF-β binding region comprises or consists of an amino acid sequence having at least 80% amino acid sequence identity to SEQ ID NO:4.

In another aspect, the present invention provides a nucleic acid encoding the TGF-β decoy receptor or CAR according to the present invention.

In another aspect, the present invention provides a vector comprising the nucleic acid of the present invention.

In another aspect, the present invention provides a cell comprising the TGF-β decoy receptor or CAR, the nucleic acid or the vector according to the present invention.

In another aspect, the present invention provides a method for producing a cell expressing a TGF-β decoy receptor or CAR, comprising introducing into a cell the nucleic acid or the vector according to the present invention, and culturing the cell under conditions suitable for expression of the nucleic acid or vector by the cell.

In another aspect, the present invention provides a cell which is obtained or obtainable by the method for producing a cell expressing a TGF-β decoy receptor or CAR according to the present invention.

In another aspect, the present invention provides a pharmaceutical composition comprising the TGF-β decoy receptor or CAR, the nucleic acid, the vector, or the cell according to the present invention, and a pharmaceutically acceptable carrier, adjuvant, excipient, or diluent.

In another aspect, the present invention provides some embodiments, the TGF-β decoy receptor or CAR, the nucleic acid, the vector, the cell or the pharmaceutical composition according to the present invention for use in a method of treating or preventing a disease or condition.

In another aspect, the present invention provides the use of the TGF-β decoy receptor or CAR, the nucleic acid, the vector, the cell or the pharmaceutical composition according to the present invention in the manufacture of a medicament for treating or preventing a disease or condition.

In another aspect, the present invention provides a method of treating or preventing a disease or condition, comprising administering to a subject a therapeutically or prophylactically effective amount of the TGF-β decoy receptor or CAR, the nucleic acid, the vector, the cell or the pharmaceutical composition according to the present invention.

In another aspect, the present invention provides a method of treating or preventing a disease or condition in a subject, comprising:
(a) isolating at least one cell from a subject;
(b) modifying the at least one cell to express or comprise the TGF-β decoy receptor or CAR, the nucleic acid, or the vector according to the present invention, and;
(c) administering the modified at least one cell to a subject.

In another aspect, the present invention provides a method of treating or preventing a disease or condition in a subject, comprising:
(a) isolating at least one cell from a subject;
(b) introducing into the at least one cell the nucleic acid or the vector according to the present invention, thereby modifying the at least one cell and;
(c) administering the modified at least one cell to a subject.

In some embodiments in accordance with various aspects of the present invention the disease or condition is a cancer.

In another aspect, the present invention provides a kit of parts comprising a predetermined quantity of the TGF-β decoy receptor or CAR, the nucleic acid, the vector, the cell or the pharmaceutical composition according to the present invention.

### DESCRIPTION

The present invention relates to TGF-β decoy receptors capable of binding to and inhibiting TGF-β mediated signalling, nucleic acids encoding such TGF-β decoy receptors and cells expressing the same. The TGF-β decoy receptors of the present invention function as decoy receptors for TGF-β. Therapeutic uses of the polypeptides, nucleic acids and cells is also described.

### TGF-β and TGF-β Mediated Signalling

Transforming growth factor β (TGF-β) is a multifunctional cytokine having three forms designated TGF-β1, TGF-β2 and TGF-β3. TGF-β1 to 3 form a subfamily of highly similar proteins within the TGF-β superfamily of cytokines.

Human TGF-β1 (NCBI Reference Sequence: NP_000651.3) is a 390 amino acid protein, whilst TGF-β2 (NCBI Reference Sequence: NP_001129071.1) is a 442 amino acid protein, and TGF-β3 (NCBI Reference Sequence: NP_001316868.1) contains 412 amino acids. Each of TGF-β1-3 have a 20-30 amino acid signal peptide at the N-terminus which is necessary for secretion, a pro-region called latency associated peptide (LAP), and a 112-114 amino acid C-terminal region that becomes the mature TGF-β molecule following proteolytic cleavage from the pro-region (Khalil et al. 1999 Microbes Infect. 1 (15): 1255-63). Mature monomeric TGF-β dimerize to produce the biologically active 25 KDa protein. TGF-β1 to 3 can form homodimers with the same type of TGF-β, or can form heterodimers with another type of TGF-β (e.g. a TGF-β1:TGF-β2 heterodimer). TGF-β comprises nine conserved cysteine residues, eight of which form disulfide bonds to form the cysteine knot structure which is characteristic of the TGF-β superfamily. The remaining cysteine is involved interacts with that of another TGF-β monomer to form the dimer. The surface-exposed region between the fifth and sixth conserved cysteine residues is the region which is least conserved between TGF-β1 to 3 proteins, and is thought to be important for receptor binding and specificity of TGF-β.

In this specification “TGF-β” refers to TGF-β from any species and includes isoforms, fragments, variants or homologues of a TGF-β from any species. In some embodiments, the TGF-β is human TGF-β, primate TGF-β, non-human primate TGF-β, rodent TGF-β, murine TGF-β, or mammalian TGF-β. In some embodiments, the TGF-β receptor is TGF-β1 (TGF-β1), TGF-β2 (TGF-β2), or TGF-β3 (TGF-β3).

TGF-β exerts its functional consequences through binding to and activating signalling through TGF-β receptors. TGF-β receptors comprising an extracellular domain having a TGF-β binding region, a single pass transmembrane domain and an intracellular domain comprising a serine/threonine kinase domain. There are three main types of TGF-β receptor; TGF-βR1 (NCBI Reference Sequence: NP_004603.1) TGF-βR2 (NCBI Reference Sequence: NP_001020018.1) and TGF-βR3 (NCBI Reference Sequence: NP_003234.2).

In this specification “TGF-β receptor” refers to a TGF-β receptor from any species and includes isoforms, fragments, variants or homologues of a TGF-β receptor from any species. In some embodiments, the TGF-β receptor is human TGF-β receptor, primate TGF-β receptor, non-human primate TGF-β receptor, rodent TGF-β receptor, murine TGF-β receptor, or mammalian TGF-β receptor. In some embodiments, the TGF-β receptor is TGF-β receptor 1 (TGF-βR1), TGF-β receptor 2 (TGF-βR2), or TGF-β receptor 3 (TGF-βR3).

Human TGF-βR1 (including the 33 amino acid signal peptide) is a 503 amino acid protein, and the mature form is 470 amino acids, comprising a 93 amino acid extracellular domain, a 21 amino acid transmembrane domain, and a 356 amino acid intracellular domain. The extracellular domain of the protein comprises the TGF-β binding region of TGF-βR1. The amino acid sequence of human TGF-βR1 (NCBI Reference Sequence: NP_004603.1) and domains thereof are shown below:

Human TGF-βR1 (NP_004603.1)
(SEQ ID NO: 1)

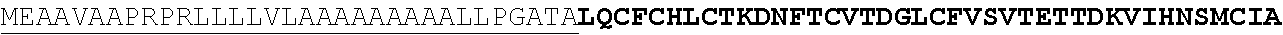

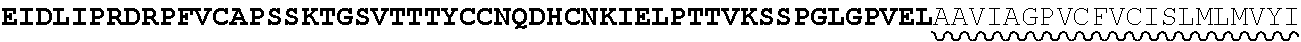

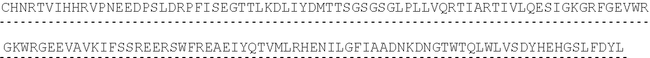

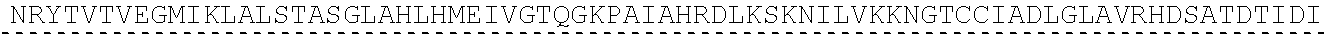

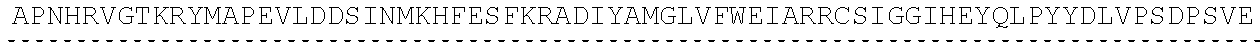

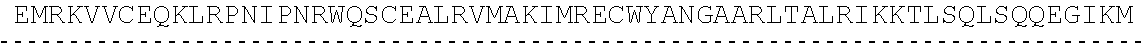

Signal peptide (residues 1-33); extracellular domain (residues 34-126); [symbol] (residues 127-147); intracellular domain (residues 148-503).

Human TGF-βR2 (including the 22 amino acid signal peptide) is a 592 amino acid protein, and the mature form is 570 amino acids, comprising a 169 amino acid extracellular domain, a 21 amino acid transmembrane domain, and a 380 amino acid intracellular domain. The extracellular domain of the protein comprises the TGF-β binding region of TGF-βR2 (Hart et al. 2002, Nat Struct Biol. 9(3):203-8; pfam08917: ecTbetaR2). The amino acid sequence of human TGF-βR2 (NCBI Reference Sequence: NP_001020018.1) and domains thereof are shown below:

Human TGF-βR2 (NP_001020018.1)
(SEQ ID NO: 2)
MGRGLLRGLWPLHIVLWTRIASTIPPHVQKSDVEMEAQKDEIICPSCNRTAHPLRHINNDMIVTDNNGAVKF

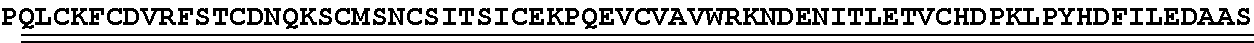

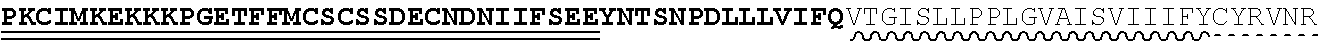

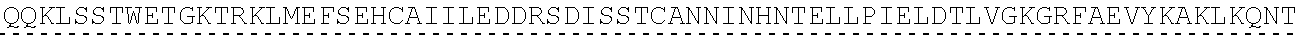

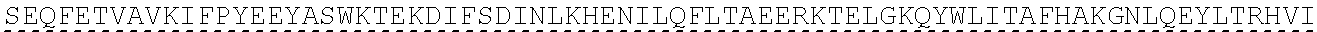

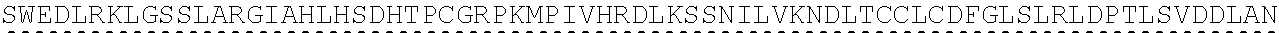

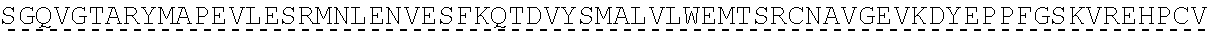

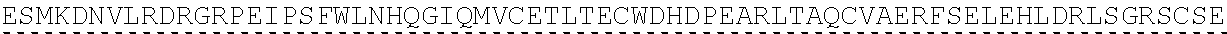

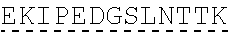

Signal peptide (residues 1-22); extracellular domain (residues 23-191); TGF-β binding region (residues 74-177); [symbol] (residues 192-212); intracellular domain (residues 213-592).

Human TGF-βR3 (including the 18 amino acid signal peptide) is a 851 amino acid protein, and the mature form is 833 amino acids, comprising a 769 amino acid extracellular domain, a 22 amino acid transmembrane domain, and a 42 amino acid intracellular domain. The extracellular domain of the protein comprises the TGF-β binding region of TGF-βR3. The amino acid sequence of human TGF-βR3 (NCBI Reference Sequence: NP_003234.2) and domains thereof are shown below:

Human TGF-βR3 (NP_003234.2)
(SEQ ID NO: 3)
MTSHYVIAIFALMSSCLATAGPEPGALCELSPVSASHPVQALMESFTVLSGCASRGTTGLPQEVHVLNLRT
AGQGPGQLQREVTLHLNPISSVHIHHKSVVFLLNSPHPLVWHLKTERLATGVSRLFLVSEGSVVQFSSANF
SLTAETEERNFPHGNEHLLNWARKEYGAVTSFTELKIARNIYIKVGEDQVFPPKCNIGKNFLSLNYLAEYLQ
PKAAEGCVMSSQPQNEEVHIIELITPNSNPYSAFQVDITIDIRPSQEDLEVVKNLILILKCKKSVNWVIKSFDV
KGSLKIIAPNSIGFGKESERSMTMTKSIRDDIPSTQGNLVKWALDNGYSPITSYTMAPVANRFHLRLENNAE
EMGDEEVHTIPPELRILLDPGALPALQNPPIRGGEGQNGGLPFPFPDISRRVWNEEGEDGLPRPKDPVIPSI
QLFPGLREPEEVQGSVDIALSVKCDNEKMIVAVEKDSFQASGYSGMDVTLLDPTCKAKMNGTHFVLESPL
NGCGTRPRWSALDGVVYYNSIVIQVPALGDSSGWPDGYEDLESGDNGFPGDMDEGDASLFTRPEIVVFNC
SLQQVRNPSSFQEQPHGNITFNMELYNTDLFLVPSQGVFSVPENGHVYVEVSVTKAEQELGFAIQTCFISPY
SNPDRMSHYTIIENICPKDESVKFYSPKRVHFPIPQADMDKKRFSFVFKPVFNTSLLFLQCELTLCTKMEKH
PQKLPKCVPPDEACTSLDASIIWAMMQNKKTFTKPLAVIHHEAESKEKGPSMKEPNPISPPIFHGLDTLTV

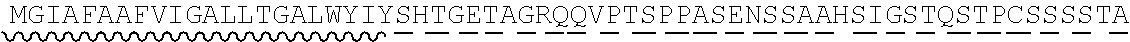

Signal peptide (residues 1-18); extracellular domain (residues 19-787); [symbol] (residues 788-809); intracellular domain (residues 810-851).

Other TGF-β binding proteins have been described. Onichtchouk et al. 1999, Nature 401(6752):480-485 describes the BMP and activing membrane-bound inhibitor (BAMBI) protein, a transmembrane glycoprotein which is closely related to the type I receptors of the TGF-β family in the extracellular domain, but which has a shorter intracellular domain with no enzymatic activity. BAMBI has been shown to inhibit TGF-β signalling by blocking interaction between TGF-βR1 and TGF-βR2. Bollard et al. 2002, Blood 99(9): 3179-3187 and Zhang et al. 2013, Gene Therapy 20,575-580 describe engineering of antigen-reactive T cells to express a dominant negative TGF-βR2 (DN-TGF-βR2), which comprise the extracellular domain of TGF-βR2 and the transmembrane region, but which lack the cytoplasmic domain required for signalling. Penafuerte et al. 2011, J Immunol. 186(12):6933-6944 describes a chimeric fusion protein of IL-2 and the soluble extracellular domain of TGF-βR2, which acts as a decoy receptor trapping active TGF-β in solution and interacting with IL-2-responsive lymphoid cells, inducing IL-2R mediated signalling. Russo et al. 2009, Int J Biochem Cell Biol 41:472-476 and Zhang et al. 2013 (supra) describe soluble TGF-β receptor molecules, comprising the extracellular TGF-β binding domain of TGF-β receptors. In some cases the soluble TGF-β receptor molecules also comprise an Fc domain.

TGF-β mediated signalling is involved in embryogenesis and tissue homeostasis, and mediates its effects through regulation of cell proliferation, differentiation, apoptosis, adhesion, invasion and the cellular microenvironment, and is described for example in Meulmeester and ten Dijke 2011, J Pathol 223: 205-218.

Active TGF-β dimers mediate signalling through the TGF-β type I and type II receptors (TGF-βR1 and TGF-βR2, respectively). TGF-βR2 is responsible for recruiting TGF-β into the heteromeric TGF-β:TGF-βR1:TGF-βR2 receptor complex, and binds to TGF-β1 and TGF-β3 with a K_{D} of ˜5 pM, and binds TGF-β3 with a K_{D} of ˜5 nM (De Crescenzo et al. 2003, J Mol Biol. 328(5):1173-83). Membrane-anchored TGF-βR3 assists TGF-β binding to TGF-βR2 (Lopez-Casillas et al., Cell 1993; 73:1435-1444). High affinity binding of TGF-β to TGF-βR2 leads to hetero-tetrameric complex formation with TGF-βR1, which in turn results in the phosphorylation of TGF-βR1 by TGF-βR2.

In most cell types TGF-βR1 transduces signalling, whilst in certain cell types ALK1 or other TGF-β superfamily type I receptors can mediate signalling responses. TGF-βR1 propagates signalling by recruitment and phosphorylation of receptor-regulated Smad proteins. Activated Smads form a complex with the common Smad (co-Smad; Smad4 in mammals) and shuttle into the nucleus where they interact with DNA-binding transcription factors to regulate transcription of target genes. Smad-independent signalling may also occur through TRAF6-TAK1-p38/JNK pathway, and TGF-βR1 also activates Erk-MAP kinase signalling through direct phosphorylation of Shc on tyrosine and serine residues (Lee et al., 2007 EMBO J 26: 3957-3967). TGF-wβR2 can also signal independently of TGF-βR1 by direct phosphorylation of Par6 (Ozdamar et al., Science; 307: 1603-1609).

As used herein, ‘TGF-β mediated signalling’ refers to signalling mediated by TGF-β and/or TGF-β receptor, fragments of TGF-β and/or TGF-β receptor, and polypeptide complexes comprising TGF-β, TGF-β receptor and/or fragments thereof.

In this specification a TGF-β receptor refers to a polypeptide capable of binding TGF-β. A TGF-β receptor may be from any species and includes isoforms, fragments, variants or homologues of a TGF-β receptor from any species. In preferred embodiments the species is human (Homo sapiens). In some embodiments the TGF-β receptor may be TGFβR1, TGF-βR2 or TGF-βR3. Isoforms, fragments, variants or homologues of a TGF-β receptor may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of a TGF-β receptor from a given species, e.g. human. Isoforms, fragments, variants or homologues of a TGF-β receptor may optionally be characterised by ability to bind TGF-β (preferably from the same species) and stimulate signal transduction in cells expressing the TGF-β receptor. A fragment of a TGF-β receptor may be of any length (by number of amino acids), although may optionally be at least 25% of the length of the mature TGF-β receptor and have a maximum length of one of 50%, 75%, 80%, 85%, 90%, 91%, 92%, 93%, 94%, 95%, 96%, 97%, 98%, or 99% of the length of the mature TGF-β receptor. A fragment of a TGF-β receptor fragment may have a minimum length of 10 amino acids, and a maximum length of one of 15, 20, 25, 30, 40, 50, 100, 110, 120, 130, 140, 150, 160, 170, 180, 190, 200, 250, 300, 400, 500, 600, 700 or 800 amino acids.

In some embodiments, the TGF-β receptor may comprise, or consist of, the TGF-β binding region of TGF-βR2. By the definition of Hart et al. 2002, Nat Struct Biol. 9(3):203-8, the TGF-β binding region of human TGF-βR2 corresponds to amino acids 74 to 177 of SEQ ID NO:2 (pfam08917: ecTbetaR2). In some embodiments, the TGF-β receptor may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of the TGF-β binding region of TGF-βR2 from a given species.

In some embodiments, the TGF-β receptor may comprise, or consist of, the extracellular domain of TGF-βR2. The extracellular domain of human TGF-βR2 corresponds to amino acids 23 to 191 of SEQ ID NO:2. In some embodiments, the TGF-β receptor may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of the extracellular domain of TGF-βR2 from a given species.

In some embodiments, the TGF-β receptor may comprise, or consist of, the extracellular domain of TGF-βR1. The extracellular domain of human TGF-βR1 corresponds to amino acids 34 to 126 of SEQ ID NO:1. In some embodiments, the TGF-β receptor may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of the extracellular domain of TGF-βR1 from a given species.

In some embodiments, the TGF-β receptor may comprise, or consist of, the extracellular domain of TGF-βR3. The extracellular domain of human TGF-βR3 corresponds to amino acids 19 to 787 of SEQ ID NO:3. In some embodiments, the TGF-β receptor may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of the extracellular domain of TGF-βR3 from a given species.

In some embodiments, TGF-β (e.g. TGF-β1, TGF-β2 or TGF-β3) is mammalian (e.g. cynomolgous, human and/or rodent (e.g. rat and/or murine) TGF-β). Isoforms, fragments, variants or homologues of TGF-β may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature TGF-β from a given species, e.g. human. Isoforms, fragments, variants or homologues of TGF-β may optionally be characterised by ability to bind a TGF-β receptor (e.g. TGF-βR1, TGF-βR2 or TGF-βR3; preferably from the same species) and stimulate signal transduction in cells expressing the TGF-β receptor. A fragment of TGF-β may be of any length (by number of amino acids), although may optionally be at least 25% of the length of mature TGF-β and may have a maximum length of one of 50%, 75%, 80%, 85%, 90%, 91%, 92%, 93%, 94%, 95%, 96%, 97%, 98%, or 99% of the length of mature TGF-β. A fragment of TGF-β may have a minimum length of 10 amino acids, and a maximum length of one of 15, 20, 25, 30, 40, 50, 100, 150, 175, 200, 225, 250, 275, 300, 325, 350, 375, 400, 425 or 440 amino acids.

### TGF-β Mediated Signalling and Disease

TGF-β/TGFβ receptor signalling is also implicated in cancer, as described for example in Nacif and Shaker 2014, J Cancer Ther 5:735-747, Meulmeester and ten Dijke 2011, J Pathol 223: 205-218, and Akhurst and Hata 2012 Nat Rev Drug Discov. 11(10): 790-811.

Mutation of TGF-β signalling components has been observed in many cancers; for example, in colon, gastric, biliary, pulmonary, ovarian, oesophageal, and head and neck carcinomas TGF-β receptor mutations are highly represented with microsatellite instability (MSI), and Smad4 mutations have been observed in pancreatic cancers and gastrointestinal tumours. TGF-β exerts tumour-suppressive effects e.g. through regulation of cell proliferation, apoptosis, and indirectly through the tumour stroma.

Whilst TGF-β exerts protective or tumor suppressive effects on epithelial cells or during the early growth-sensitive stages of tumorigenesis, later in tumor development carcinoma cells are refractory to TGF-β-mediated growth inhibition, and during the late stages of malignancy tumor progression is driven by TGF-β overload.

In the tumor microenvironment TGF-β stimulates tumor progression via pro-tumorigenic effects on vascular and immune cells and fibroblasts, promoting invasion and metastasis. Once tumor cells have undergone genetic and/or epigenetic changes that attenuate the growth suppressive effects of TGF-β, TGF-β1 can drive malignant progression and metastasis (Connolly et al. 2012, Journal of Biological Sciences, 8, 964-978). Different types of tumours such as gliomas and breast and prostate cancer seem to acquire mutations preferentially not in the core components of TGF-β signalling and retain the ability to exploit TGF-β signalling to induce pathways that promote epithelial-to-mesenchymal transition (EMT), tumour invasion, metastatic dissemination, and evasion of the immune system.

Torre-Amione et al. 1990, Proc Natl Acad Sci USA 87: 1486-1490 reported the ability of TGF-β to inhibit tumour-induced CD8+ cytolytic T-lymphocyte (CTL)-mediated rejection of a murine tumour, and growth and metastasis of melanoma or lymphoma cell lines is reduced in mice expressing a dominant negative form of TGF-βR2 (DN-TGF-βR2) in all T-cells (Gorelik et al. 2001 Nature Med 7:1118-1122). TGF-β has been shown to repress production of pro-apoptotic factors in CTLs such as perforin, granzyme A, granzyme B, FAS ligand, and interferon-γ (Thomas et al. 2005 Cancer Cell 8: 369-380). Bollard et al. 2002, Blood 99(9):3179-3187 reported that EBV-specific CTLs engineered to express DN-TGF-βR2 were resistant to anti-proliferative and anti-cytotoxic effects of TGF-β, and continued to secrete cytokines in response to antigenic stimulation; such CTLs would have a selective functional and survival advantage (Bollard et al. 2002, Blood 99(9):3179-3187). T cells engineered to express soluble TGFβ-2R or a DN-TGF-βR2 have been shown to be resistant to TGF-β induced Smad2 phosphorylation in vitro, and expression of DN-TGF-βR2 in antigen-specific CD8+ and CD4+ T cells has been shown to be improve tumour treatment efficiency in a melanoma tumour model (Zhang et al. 2013, Gene Therapy 20, 575-580)

TGF-β has also been shown to repress the activity of natural killer (NK) cells; inhibition of TGF-β using neutralizing anti-TGF-β antibodies increased NK cell activity and thus resulted in suppression of metastasis formation of an inoculated breast carcinoma cell line (Arteaga et al. 1993, J Clin Invest 92: 2569-2576). TGF-β inhibits NK-mediated cytotoxicity (and antitumour activity) through transcriptional repression of NKG2D and NKp30 (Castriconi et al. 2003, Proc Natl Acad Sci USA 100: 4120-4125; Kopp et al. 2009 Cancer Res 69:7775-7783).

TGF-β mediated signalling is also important in in the pathology of other disease/conditions, as described for example in Nacif and Shaker 2014 (supra) and Akhurst and Hata 2012 (supra). TGF-β signalling is implicated in the pathology of diseases characterised by fibrosis, such as idiopathic pulmonary fibrosis (IPF), renal fibrosis, cardiac fibrosis (e.g. fibrosis associated with myocardial infarction, ischaemic, dilated and hypertrophic cardiomyopathies and congestive heart failure), scleroderma, myelodysplastic syndrome (MDS), restenosis following coronary artery bypass or angioplasty, Marfan syndrome, post-operative scarring in ocular conditions (e.g. following trabeculectomy for treatment of glaucoma, or following corneal surgery), diabetes and obesity.

### TGF-β Decoy Receptors

The present invention provides a TGF-β decoy receptor. The TGF-β decoy receptor is a peptide/polypeptide or plurality (e.g. non-covalent complex) thereof which is capable of binding to TGF-β (e.g. TGF-β1, TGF-β2 and/or TGF-β3). By ‘receptor’ we include fragments and derivatives thereof. A TGF-β decoy receptor' is a peptide/polypeptide or plurality thereof capable of binding to TGF-β in the manner of a cytokine receptor binding to its ligand, and which is unable to signal. The decoy receptor therefore acts as an inhibitor of TGF-β mediated signalling, through binding to TGF-β and making ligand unavailable for binding to signalling-competent TGF-β receptor.

TGF-β decoy receptors according to the present invention may be provided in isolated form.

A TGF-β decoy receptor is not an antibody or an antigen-binding fragment of an antibody which is specific for TGF-β. A TGF-β decoy receptor lacks sequences encoding heavy and light chain complementarity determining regions (CDRs) and/or heavy chain and light chain variable regions of an antibody or antigen binding fragment capable of specific binding to TGF-β.

As used herein, a “peptide” is a chain of two or more amino acid monomers linked by peptide bonds. A peptide typically has a length in the region of about 2 to 50 amino acids. A “polypeptide” is a polymer chain of two or more peptides. Polypeptides typically have a length greater than about 50 amino acids.

TGF-β decoy receptors according to the present invention bind to TGF-β (e.g. TGF-β1, TGF-β2 and/or TGF-β3). In some embodiments, the TGF-β decoy receptor binds to human TGF-β (e.g. human TGF-β1, TGF-β2 and/or TGF-β3). In some embodiments, the TGF-β decoy receptor binds to non-human primate TGF-β1, TGF-β2 and/or TGF-β3. In some embodiments, the TGF-β decoy receptor binds to murine TGF-β1, TGF-β2 and/or TGF-β3.

In some embodiments, the TGF-β decoy receptors bind to TGF-β containing molecules/complexes, such as a TGF-β:TGF-β receptor complex. For example, the TGF-β decoy receptor may bind e.g. to a complex of a TGF-β (e.g. TGF-β1, TGF-β2 or TGF-β3) and a TGF-β receptor (e.g. TGF-βR1, TGF-βR2 or TGF-βR3).

TGF-β decoy receptors according to the present invention may bind TGF-β and TGF-β containing complexes, and thereby make these species unavailable for binding to TGF-β receptors (e.g. endogenous TGF-β receptors).

The TGF-β decoy receptors according to the present invention are signalling incompetent. The TGF-β decoy receptors preferably lack an amino acid sequence required for signal transduction by a TGF-β receptor. In some embodiments, the TGF-β decoy receptors lack amino acid sequence encoding a functional catalytic domain of a TGF-β receptor; for example, the TGF-β decoy receptor may lack amino acid sequence encoding a functional serine/threonine kinase domain. In some embodiments, the TGF-β decoy receptors lack amino acid sequence necessary for recruitment or activation of factors necessary for signal transduction by a TGF-β receptor.

As such, the present TGF-β decoy receptors act as ‘decoy’ receptors for TGF-β and TGF-β containing complexes, much in the same way that the dominant negative TGF-βR2 (i.e. DN-TGF-βR2) described e.g. in Bollard et al. 2002, Blood 99(9): 3179-3187 and Zhang et al. 2013, Gene Therapy 20, 575-580 acts as a decoy receptor for TGF-β.

As used herein ‘dominant negative TGF-βR2’ and ‘DN-TGF-βR2’ refer to species comprising the TGF-β binding region of TGF-βR2 and the transmembrane domain of TGF-βR2, and lacking a functional kinase domain.

TGF-β mediated signalling by cells expressing the TGF-β decoy receptors of the present invention is reduced as compared to the level of signalling by cells not expressing TGF-β decoy receptors of the present invention, e.g. following treatment with TGF-β.

The TGF-β decoy receptors according to the present invention preferably bind to TGF-β through one or more TGF-β binding domains. The TGF-β binding domains are, or are derived from or homologous to, the TGF-β binding domains of naturally occurring receptor molecules for TGF-β. For example, the TGF-β decoy receptors of the present invention may comprise, or consist of, one or more TGF-β binding domains which are derived from or homologous to the TGF-β binding domains of TGF-βR1, TGF-βR2 and/or TGF-βR3.

As explained herein, TGF-β and complexes containing TGF-β are capable of activating TGF-β mediated signalling through binding to TGF-β receptors expressed on the surface of cells. The TGF-β decoy receptors of the present invention are able to bind to TGF-β and TGF-β containing species such as to inhibit the ability of those species to interact with cell membrane bound TGF-β receptor, and thereby inhibit TGF-β mediated signalling.

This is preferably achieved through binding of the TGF-β decoy receptor to the region of TGF-β which is required for binding to TGF-β receptor (e.g. TGF-βR1, TGF-βR2 and/or TGF-βR3), e.g. endogenously expressed TGF-β receptor.

That is, TGF-β decoy receptors reduce the amount of TGF-β and TGF-β containing species available to bind to and activate signalling through functionally-competent TGF-β receptors expressed by cells (e.g. TGF-βR1, TGF-βR2 and/or TGF-βR3).

In some embodiments, the TGF-β decoy receptor is capable of binding to TGF-β in the region of TGF-β which is bound by TGF-βR1, TGF-βR2 and/or TGF-βR3. In some embodiments, the TGF-β decoy receptor is capable of binding to TGF-β in the same region of TGF13, or an overlapping region of TGFβ, as the region of TGF-β which is bound by TGF-βR1, TGF-βR2 and/or TGF-βR3. Ability to bind to TGF-β in the same region or an overlapping region of as the region bound by TGF-βR1, TGF-βR2 and/or TGF-βR3 can be analysed using a competitive binding assay, such as a competition ELISA. In such assay, observation of a reduction/decrease in the level of interaction between TGF-β and TGF-β receptor (e.g. TGF-βR1, TGF-βR2 and/or TGF-βR3) in the presence of or following incubation of one or both of the interaction partners with the TGF-β decoy receptor, as compared to the level of interaction in the absence of the TGF-β decoy receptor indicates that the TGF-β decoy receptor binds to the same region or overlapping region of TGF-β as the region bound by the TGF-β receptor. Whether a TGF-β decoy receptor according to the present invention binds to TGF-β in the same or overlapping region of TGF-β as the region bound by TGF-βR1, TGF-βR2 and/or TGF-βR3 can also be determined by analysis of interaction using various methods well known in the art, including X-ray co-crystallography analysis of receptor-ligand complexes, peptide scanning, mutagenesis mapping, hydrogen-deuterium exchange analysis by mass spectrometry, phage display and proteolysis-based ‘protection’ methods. Such methods are described, for example, in Gershoni et al., BioDrugs, 2007, 21(3):145-156, which is hereby incorporated by reference in its entirety.

In some embodiments, the TGF-β decoy receptor comprises an amino acid sequence corresponding to the TGF-β binding region of a TGF-β receptor. Herein, “an amino acid sequence corresponding to the TGF-β binding region of a TGF-β receptor” may be the amino acid sequence of the TGF-β binding region of a TGF-β receptor, or an amino acid sequence which is capable of binding to TGF-β and having at least 60%, e.g. one of at least 65%, 70%, 75%, 80%, 85%, 90%, 91%, 92%, 93%, 94%, 95%, 96%, 97%, 98% or 99% sequence identity to the amino acid sequence of the TGF-β binding region of a TGF-β receptor. The TGF-β receptor and TGF-β may be from any species, and includes isoforms, fragments, variants or homologues from any species. In some embodiments, the TGF-β receptor is TGF-βR1, TGF-βR2 or TGF-βR3. In some embodiments, the TGF-β is TGF-β1, TGF-β2 or TGF-β3.

In some embodiments, the TGF-β decoy receptor comprises the TGF-β binding region of a TGF-β receptor. In some embodiments, the TGF-β decoy receptor comprises an amino acid sequence having at least 70% sequence identity to the TGF-β binding region of a TGF-β receptor.

The TGF-β binding region of a given TGF-β receptor can be identified by routine methods which are well known to the skilled person, e.g. by comparison with known TGF-β binding regions, and/or by analysis of the ability of peptides/polypeptide fragments of the TGF-β receptor to bind to TGF-β, e.g. by ELISA assay.

The TGF-β binding region of TGF-βR2 according to Hart et al. 2002, Nat Struct Biol. 9(3):203-8 (pfam08917: ecTbetaR2) corresponds to positions 74 to 177 of human TGF-βR2 (NCBI Reference Sequence: NP 001020018.1) shown in SEQ ID NO:2, that is:

(SEQ ID NO: 4)

QLCKFCDVRFSTCDNQKSCMSNCSITSICEKPQEVCVAVWRKNDENITLE
TVCHDPKLPYHDFILEDAASPKCIMKEKKKPGETFFMCSCSSDECNDNII
FSEE

The skilled person is well able to identify the TGF-β binding region for a given homologue of human TGF-βR2 by routine methods.

In some embodiments the TGF-β decoy receptor comprises an amino acid sequence having at least 60%, 65%, 70%, 75%, 80%, 85%, 90%, 91%, 92%, 93%, 94%, 95%, 96%, 97%, 98%, 99% or 100% sequence identity to the TGF-β binding region of TGF-βR1, TGF-βR2 or TGF-βR3. In some embodiments the TGF-β decoy receptor comprises an amino acid sequence having greater than 70%, 75%, 80%, 85%, 90%, 91%, 92%, 93%, 94%, 95%, 96%, 97%, 98%, 99% or 100% sequence identity to the TGF-β binding region of TGF-βR1, TGF-βR2 or TGF-βR3.

In some embodiments the TGF-β decoy receptor comprises an amino acid sequence having at least 60%, 65%, 70%, 75%, 80%, 85%, 90%, 91%, 92%, 93%, 94%, 95%, 96%, 97%, 98%, 99% or 100% sequence identity to the TGF-β binding region of TGF-βR2. In some embodiments the TGF-β decoy receptor comprises an amino acid sequence having greater than 70%, 75%, 80%, 85%, 90%, 91%, 92%, 93%, 94%, 95%, 96%, 97%, 98%, 99% or 100% sequence identity to the TGF-β binding region of TGF-βR2.

In some embodiments the TGF-β decoy receptor comprises an amino acid sequence having at least 60%, 65%, 70%, 75%, 80%, 85%, 90%, 91%, 92%, 93%, 94%, 95%, 96%, 97%, 98%, 99% or 100% sequence identity to SEQ ID NO:4. In some embodiments the TGF-β decoy receptor comprises an amino acid sequence having greater than 70%, 75%, 80%, 85%, 90%, 91%, 92%, 93%, 94%, 95%, 96%, 97%, 98%, 99% or 100% sequence identity to SEQ ID NO:4.

Sequence identity between amino acid sequences can be determined by methods known to the person skilled in the art. For example, to determine the percent identity of two amino acid sequences, the sequences to be compared can be aligned (e.g., gaps can be introduced in one or both of a first and a second amino acid sequence for optimal alignment), and the amino acids at corresponding positions can be compared. When a position in the first sequence is occupied by the same amino acid as the corresponding position in the second sequence, the sequences have “identity” at that position. The percent identity between two sequences is a function of the number of identical positions, taking into account the number of gaps, and the length of each gap, which need to be introduced for optimal alignment of the sequences. Sequence identity is preferably determined over the full length of the amino acid sequences being compared.

In some embodiments, the TGF-β decoy receptor comprises an amino acid sequence having at least 70% sequence identity to the TGF-β binding region of a TGF-β decoy receptor as described herein, e.g. a TGF-β decoy receptor as shown in FIG. 1.

The TGF-β decoy receptors shown in FIG. 1 comprise a region derived from TGF-βR2, which comprises a TGF-β binding region. The TGF-βR2-derived region of the TGF-β decoy receptors of FIG. 1 is shown below:

TGF-βR2-derived region comprising the TGF-β binding region:

(SEQ ID NO: 9)

MGRGLLRGLWPLHIVLWTRIASTIPPHVQKSDVEMEAQKDEIICPSCNRT
AHPLRHINNDMIVTDNNGAVKFPQLCKFCDVRFSTCDNQKSCMSNCSITS
ICEKPQEVCVAVWRKNDENITLETVCHDPKLPYHDFILEDAASPKCIMKE
KKKPGETFFMCSCSSDECNDNIIFSEEYNTSNP

Residues 1 to 22 thereof represent the signal peptide for TGF-βR2, and so the mature sequence of SEQ ID NO:9 is:

(SEQ ID NO: 10)

TIPPHVQKSDVEMEAQKDEIICPSCNRTAHPLRHINNDMIVTDNNGAVKF
PQLCKFCDVRFSTCDNQKSCMSNCSITSICEKPQEVCVAVWRKNDENITL
ETVCHDPKLPYHDFILEDAASPKCIMKEKKKPGETFFMCSCSSDECNDNI
IFSEEYNTSNP

In some embodiments the TGF-β decoy receptor comprises an amino acid sequence having at least 60%, 65%, 70%, 75%, 80%, 85%, 90%, 91%, 92%, 93%, 94%, 95%, 96%, 97%, 98%, 99% or 100% sequence identity to SEQ ID NO:9 or 10. In some embodiments the TGF-β decoy receptor comprises an amino acid sequence having greater than 70%, 75%, 80%, 85%, 90%, 91%, 92%, 93%, 94%, 95%, 96%, 97%, 98%, 99% or 100% sequence identity to SEQ ID NO:9 or 10.

The TGF-β decoy receptor of the present invention comprises a cell membrane anchor region. As used herein, a ‘cell membrane anchor region’ is a region providing for anchoring the TGF-β decoy receptor to the cell membrane of a cell expressing the TGF-β decoy receptor. ‘Anchoring’ may be reversible or irreversible.

In some embodiments, the cell membrane anchor region may comprise or consist of an amino acid sequence. In some embodiments, the cell membrane anchor region may comprise or consist of a lipid moiety.

Anchoring may be achieved directly through non-covalent or covalent association of the cell membrane anchor region with the cell membrane or a component thereof (e.g. phospholipid). In some embodiments, anchoring may be achieved indirectly through covalent or non-covalent association of the cell membrane anchor region with a membrane-bound factor or a factor localised to the cell membrane (e.g. a peripheral or integral cell membrane-associated protein).

The cell membrane anchor region may provide for anchoring directly through covalent or non-covalent association of the region with the cell membrane/component thereof/factor.

In some embodiments, the cell membrane anchor region may provide for anchoring by encoding a signal sequence directing processing (e.g. post-translational modification) of the receptor, resulting in covalent or non-covalent association with the cell membrane/component thereof/factor. For example, the cell membrane anchor region may comprise or consist of a glycosylphosphatidylinositol (GPI) signal sequence directing processing of the receptor to attach a GPI anchor.

The cell membrane anchor region preferably is not a transmembrane domain. The TGF-β decoy receptor of the present invention preferably lacks a transmembrane domain. That is, the TGF-β decoy receptor lacks an amino acid sequence encoding a transmembrane domain. Herein, a ‘transmembrane domain’ refers to a sequence of amino acids forming a three-dimensional structure which is thermodynamically stable in a biological membrane, e.g. a cell membrane. A transmembrane domain is typically capable of spanning the lipid bilayer of the cell membrane, e.g. as a hydrophobic alpha helix or beta barrel. Transmembrane domains are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as TMHMM (Krogh et al., 2001 J Mol Biol 305: 567-580).

In some embodiments, the cell membrane anchor region according of the TGF-β decoy receptor of the present invention may be a lipid anchor region. In some embodiments, a lipid anchor region comprises or consists of a lipid anchor (e.g. a GPI anchor). In some embodiments, a lipid anchor region comprises or consists of a lipid anchor signal sequence.

As used herein, a ‘lipid anchor’ refers to a moiety capable of associating (e.g. covalently) with the lipid component of a biological membrane (e.g. cell membrane). Through such association, a protein having a lipid anchor attached thereto is ‘anchored’ in the cell membrane. Examples of lipid anchors include isoprenyl, myristoyl, palmitoyl, fatty acyl, diacylglycerol, steroyl, phospholipid and glycosylphosphatidyl inositol (GPI) anchors.

In some embodiments, the lipid anchor region comprises or consists of a lipid anchor signal sequence. Herein, a ‘lipid anchor signal sequence’ refers to an amino acid sequence directing processing of a protein to attach a lipid anchor. Following such processing the TGF-β decoy receptor comprises a lipid anchor.

A lipid anchor typically comprises a lipophilic group. Lipid anchors, lipophilic groups thereof and modification of proteins to attach lipid anchors is described for example in Resh 2013, Curr Biol. 23(10): R431R435, which is hereby incorporated by reference in its entirety.

A lipid anchor may comprise or consist of an isoprenyl, myristoyl, palmitoyl, fatty acyl, diacylglycerol, steroyl, or phospholipid group, or a glycosylphosphatidyl inositol (GPI) anchor.

An isoprenyl group may comprise 15 (farnesyl) or 20 (geranylgeranyl) carbon atoms attached to a cysteine residue of the protein at the C-terminus, via a thioether bond. The carboxyl group of the cysteine may also be methylated. The protein having an isoprenyl (e.g. farnesyl or geranylgeranyl) group covalently attached thereto may be referred to as a prenylated protein.

A myristoyl group is a 14 carbon, saturated fatty acyl group which may be attached to a glycine residue via an amide bond. The protein having a myristol group covalently attached thereto may be referred to as a myristoylated protein.

A palmitoyl group is a 16 carbon, saturated fatty acyl group which may be attached to a cysteine or residue of the protein via a thioether bond, or which may be attached to a serine or threonine residue via an ester bond. The protein having a palmitoyl group covalently attached thereto may be referred to as a palmitoylated protein.

Fatty acyl and 1,2-diacylglyceryl groups may be attached to a cysteine residue of the protein via amide and thioether linkages, respectively.

In some embodiments, the TGF-β decoy receptor may be a GPI-anchored, prenylated, myristoylated, palmitoylated, fatty acylated, diacylglycerylated, stearoylated or phospholipidated protein.

In some embodiments, the lipid anchor signal sequence may provide for modification of the TGF-β decoy receptor to attach a isoprenyl, myristoyl, palmitoyl, fatty acyl, diacylglycerol, stearoyl, or phospholipid group, or glycosylphosphatidyl inositol (GPI) anchor to the receptor. Following processing the TGF-β decoy receptor directed by the lipid anchor signal sequence the receptor may comprise a lipid or lipid-containing moiety.

In some embodiments, the ‘lipid anchor signal sequence’ is a GPI anchor signal sequence. Accordingly, in some embodiments the lipid anchor of the present invention is a GPI anchor.

A ‘GPI signal sequence’ is an amino acid sequence directing modification of a protein to attach a GPI anchor to the protein. GPI signal sequences direct post-translational modification of proteins for the covalent attachment of a GPI anchor to the C terminus of the protein. GPI anchors and their attachment to proteins is described, for example, in Vidal C. J. (Ed.) Protein Reviews 13, Springer Science 2011, at Chapter 2 entitled “GPI-Anchored Proteins in Health and Disease”, which is hereby incorporated by reference in its entirety.

Herein, a ‘GPI anchor’ refers to a moiety comprising a phosphatidylinositol group linked via a glycosidic bond to a tetrasaccharide glycan core, which in turn is attached to phosphoethanolamine.

The tetrasaccharide glycan core may be a trimannosyl-nonacetylated glucosamine (Mani-GlcN) core. The lipid tails of the phosphatidylinositol group can be e.g. acyl or alkyl fatty acids, or ceramide.

GPI anchors may be covalently attached to the alpha-carboxyl group at the C-terminus of the protein through a phosphoethanolamine bridge to the core glycan: -6mannose(α1-2)mannose(α 1-6)mannose(α1-4)glucosamine(α1-6)myo-inositol, which is in turn linked through the inositol ring by a phosphodiester bridge to a lipid moiety. The lipid moiety can vary, but in most mammals this is 1-alkyl-2-acylglycerol. The core glycan can be modified by the addition of ethanolamine and sugar side chains, the inositol ring can be modified with lipid groups, and the fatty acids can also be remodelled.

GPI signal sequences which direct addition of GPI anchors to proteins lie at the C-terminus of the amino acid chain. The sequences typically comprise 15-25 amino acid residues, with a stretch of hydrophobic residues at the C-terminal end. The C-terminal hydrophobic sequence is preceded by a consensus sequence that directs GPI anchor addition: ω, ω+1 and ω+2, wherein ω=an amino acid possessing a small side chain (e.g. Ala, Asn, Asp, Cys, Gly or Ser), ω+1=any residue except Pro or Trp, and wherein ω+2=Gly or Ala (or occasionally Ser or Thr).

The preassembled GPI anchor is attached in the ER to the C-terminal side of the w residue by the transamidase enzyme following cleavage of the polypeptide between ω and ω+1 residues. Transamidase is a multiprotein complex comprising the catalytic component Gpi8P, and the other components Gaa1p, Gpi16p (PIG-T) and Gpi17p (PIG-S) (Fraering et al. 2001 Molecular Biology of the Cell, 12, 3295-3306).

Following attachment of the GPI anchor, the GPI anchored protein lacks the sequence of amino acids C-terminal to the ω residue. Prediction tools for GPI modification sites are available, including e.g. big-PI Prediction Server (Eisenhaber et al. Protein Engineering (1998) 11, No.12, 1155-1161; Sunyaev et al. Protein Engineering (1999) 12, No.5, 387-394; Eisenhaber et al. JMB (1999) 292 (3), 741-758; and Eisenhaber et al. TIBS (2000) 25 (7), 340-341). Such tools can be used to identify the ω residue.

In FIG. 1, the ω residue as predicted using the big-PI Prediction Server for the different GPI signal sequences is indicated. FIG. 2 shows the structure of the mature GPI-anchored proteins following processing of the amino acid sequences shown in FIG. 1 to attach a GPI anchor.

In some embodiments, the TGF-β decoy receptor of the present invention comprises a GPI signal sequence, e.g. prior to processing to attach a GPI anchor. A GPI signal sequence is any sequence capable of directing post-transitional processing to attach a GPI anchor.

In some embodiments, the TGF-β decoy receptor comprises a GPI signal sequence up to and including the ω residue, e.g. following processing to attach a GPI anchor.

GPI signal sequences and the w residues thereof are known for many GPI-anchored proteins, and recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as big-PI Prediction Server (supra).

In some embodiments, the TGF-β decoy receptor of the present invention comprises a GPI signal sequence (e.g. prior to processing to attach a GPI anchor), or comprises a GPI signal sequence up to and including the w residue (e.g. following processing to attach a GPI anchor), corresponding to the GPI signal sequence of a GPI-anchored protein. In some embodiments, the GPI-anchored protein is one of: homing cell adhesion molecule (HCAM; CD44) neural cell adhesion molecule (NCAM; CD56), 5′Nucleotidase (CD73), decay accelerating factor (DAF; CD55), CD90 (Thyl), acetylcholinesterase (AchE), intestinal alkaline phosphatase, placental alkaline phosphatase, MAC-inhibitory protein (CD59), CD14, CD16, CD24, CD28, CD48, CDw52, CD58, CD66a, CD66c, CD66d, CD66E, CD67, CD87, CD108, CD157, acetylcholinesterase (AchE), Urokinase type plasminogen activating receptor (uPAR), JMH protein, GDNFR, CNTFR, TAG-1, PrP, Glypican protein, Semaphorin 7, CEA, GFR, Ly6G, transferrin receptor, Contactin (F3) and T-cadherin. The protein may be from any species, and includes isoforms, fragments, variants or homologues from any species.

In some embodiments, the GPI-anchored protein is one of CD48 (BLAST-1), CD44 (HCAM), CD55 (DAF), placental alkaline phosphatase and CD90 (Thy1).

Herein, a ‘GPI signal sequence corresponding to a reference GPI signal sequence’ is an amino acid sequence which is capable of directing attachment of a GPI anchor to the protein comprising the sequence, and which has at least 60%, e.g. one of at least 65%, 70%, 75%, 80%, 85%, 90%, 91%, 92%, 93%, 94%, 95%, 96%, 97%, 98%, 99% or 100% sequence identity to the reference GPI signal sequence.

In some embodiments, the TGF-β decoy receptor of the present invention comprises a GPI signal sequence (e.g. prior to processing to attach a GPI anchor), or comprises a GPI signal sequence up to an including the ω residue (e.g. following processing to attach a GPI anchor), corresponding to the GPI signal sequence of one of SEQ ID NOs:15 to 18 (ω residue is underlined):

CD48 GPI:

(SEQ ID NO: 15)

DVCSPPCTLARSFGVEWIASWLVVTVPTILGLLLT
Sequence following processing to attach GPI:

(SEQ ID NO: 19)

DVCSPPCTLARS
CD55 GPI:

(SEQ ID NO: 16)

HETTPNKGSGTTSGTTRLLSGHTCFTLTGLLGTLVTMGLLT
Sequence following processing to attach GPI:

(SEQ ID NO: 20)

HETTPNKGSGTTS
PLAC ALKPHOS GPI:

(SEQ ID NO: 17)

TACDLAPPAGTTDAAHPGPSVVPALLPLLAGTLLLLGTATAP
Sequence following processing to attach GPI:

(SEQ ID NO: 21)

TACDLAPPAGTTD
CD90 GPI:

(SEQ ID NO: 18)

NVTVLRDKLVKCEGISLLAQNTSWLLLLLLSLSLLQATDFMSL
Sequence following processing to attach GPI:

(SEQ ID NO: 22)

NVTVLRDKLVKC

The CD48-derived sequence of SEQ ID NO:15 corresponds to positions 4 to 35 of SEQ ID NO:15. The CD48-derived sequence of SEQ ID NO:19 corresponds to positions 4 to 12 of SEQ ID NO:19.

In some embodiments, the TGF-β decoy receptor of the present invention comprises a GPI signal sequence (e.g. prior to processing to attach a GPI anchor) having at least 60%, e.g. one of at least 65%, 70%, 75%, 80%, 85%, 90%, 91%, 92%, 93%, 94%, 95%, 96%, 97%, 98%, 99% or 100% sequence identity to one of SEQ ID NOs:15 to 18, or positions 4 to 35 of SEQ ID NO:15.

In some embodiments, the TGF-β decoy receptor of the present invention comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to one of SEQ ID NOs:19 to 22 or positions 4 to 12 of SEQ ID NO:19, and a GPI anchor (e.g. following processing to attach a GPI anchor).

In some embodiments, the TGF-β decoy receptor of the present invention may comprise or consist of a structure as shown in FIG. 1 or 2.

In some embodiments the TGF-β decoy receptor comprises a TGF-β binding region comprising an amino acid sequence having at least 60%, 65%, 70%, 75%, 80%, 85%, 90%, 91%, 92%, 93%, 94%, 95%, 96%, 97%, 98%, 99% or 100% sequence identity to SEQ ID NO:9 or 10; and a GPI signal sequence (e.g. prior to processing to attach a GPI anchor) having at least 60%, e.g. one of at least 65%, 70%, 75%, 80%, 85%, 90%, 91%, 92%, 93%, 94%, 95%, 96%, 97%, 98%, 99% or 100% sequence identity to one of SEQ ID NOs:15 to 18, or positions 4 to 35 of SEQ ID NO:15.

In some embodiments the TGF-β decoy receptor comprises a TGF-β binding region comprising an amino acid sequence having at least 60%, 65%, 70%, 75%, 80%, 85%, 90%, 91%, 92%, 93%, 94%, 95%, 96%, 97%, 98%, 99% or 100% sequence identity to SEQ ID NO:9 or 10; and an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to one of SEQ ID NOs:19 to 22 or positions 4 to 12 of SEQ ID NO:19 and a GPI anchor (e.g. following processing to attach a GPI anchor).

In some embodiments, the TGF-β decoy receptor of the present invention comprises a TGF-β binding region, and a GPI signal sequence, and comprises or consists of an amino acid sequence (e.g. prior to processing to attach a GPI anchor) having at least 60%, e.g. one of at least 65%, 70%, 75%, 80%, 85%, 90%, 91%, 92%, 93%, 94%, 95%, 96%, 97%, 98%, 99% or 100% sequence identity to one of SEQ ID NOs:5 to 8.

In some embodiments, the TGF-β decoy receptor of the present invention comprises or consists of a TGF-β binding region and a GPI anchor, and comprises an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to one of SEQ ID NOs:11 to 14.

GPI anchored proteins are orientated in the cell membrane with the amino acid sequence N-terminal to the w residue in the extracellular space (i.e. the apical surface). TGF-β decoy receptors according to the present invention are provided with the TGF-β binding region N-terminal to the GPI signal sequence.

In some embodiments, the TGF-β decoy receptor according to the present invention may comprise a leader sequence (also known as a signal peptide or signal sequence). Leader sequences normally consist of a sequence of 5-30 hydrophobic amino acids, which form a single alpha helix. Secreted proteins and proteins expressed at the cell surface often comprise leader sequences. The leader sequence may be present at the N-terminus of the TGF-β decoy receptor, and may be present in the newly synthesized receptor (e.g. prior to processing to remove the leader sequence). The leader sequence provides for efficient intracellular trafficking of the TGF-β decoy receptor. Leader sequences are often removed by cleavage, and thus are not comprised in the mature TGF-β decoy receptor.

Leader sequences are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8: 785-786) or Signal-BLAST (Frank and Sippl, 2008 Bioinformatics 24: 2172-2176).

In some embodiments, the leader sequence of the TGF-β decoy receptor of the present invention comprises, or consists of, an amino acid sequence having at least 80%, 85% 86%, 87%, 88%, 89%, 90%, 91%, 92%, 93%, 94%, 95%, 96%, 97%, 98%, 99%, or 100% sequence identity to the amino acid sequence of SEQ ID NO:23:

TGF-βR2 leader sequence:

(SEQ ID NO: 23)

MGRGLLRGLWPLHIVLWTRIAS

In some embodiments, the TGF-β decoy receptor may comprises one or more linkers between the TGF-β binding region and lipid anchor region. The linker may comprise or consist of an amino acid sequence, and may be covalently bonded (e.g. by peptide bonds) to ends of amino acid sequences of the TGF-β binding region and lipid anchor region.

The linker may be a peptide or polypeptide linker. The linker may be a flexible linker. Amino acid sequences of flexible linkers are known to the skilled person, and are described, for example in Chen et al., Adv Drug

Deliv Rev (2013) 65(10): 1357-1369, which is hereby incorporated by reference in its entirety. In some embodiments the flexible linker sequence comprises serine and glycine residues. In some embodiments the linker is a peptide/polypeptide consisting of an amino acid sequence of 1-100, 1-50, 1-20, 1-10 or 1-5 amino acid residues.

TGF-β decoy receptors according to the present invention are distinct from naturally-occurring TGF-β binding molecules, e.g. a naturally occurring receptor for TGF-β.

TGF-β decoy receptors according to the present invention are also distinct from DN-TGF-βR2; DN-TGF-βR2 comprises the transmembrane domain of TGF-βR2, whereas TGF-β decoy receptors according to the present invention lack a transmembrane domain.

TGF-β decoy receptors according to the present invention are also distinct from soluble TGF-βR2 species disclosed in the prior art, which lack a cell membrane anchor region. By contrast, the TGF-β decoy receptors according to the present invention comprise a cell membrane anchor region, such as a lipid anchor region.

In some embodiments, the TGF-β decoy receptor lacks amino acid sequence corresponding to the transmembrane and/or intracellular domain of a TGF-β receptor (e.g. TGF-βR1, TGF-βR2 or TGF-βR3).

Herein, an amino acid sequence which ‘corresponds’ to a reference region or sequence of a given peptide/polypeptide has at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of the reference region/sequence.

In some embodiments, the TGF-β decoy receptor lacks amino acid sequence corresponding to residues 127-147 of SEQ ID NO:1. In some embodiments, the TGF-β decoy receptor lacks amino acid sequence corresponding to residues 192-212 of SEQ ID NO:2. In some embodiments, the TGF-β decoy receptor lacks amino acid sequence corresponding to residues 788-809 of SEQ ID NO:3.

In some embodiments, the TGF-β decoy receptor lacks amino acid sequence corresponding to residues 127-147 of SEQ ID NO:1. In some embodiments, the TGF-β decoy receptor lacks amino acid sequence corresponding to residues 192-212 of SEQ ID NO:2. In some embodiments, the TGF-β decoy receptor lacks amino acid sequence corresponding to residues 788-809 of SEQ ID NO:3.

In some embodiments, the TGF-β decoy receptor lacks amino acid sequence corresponding to residues 148-503 of SEQ ID NO:1. In some embodiments, the TGF-β decoy receptor lacks amino acid sequence corresponding to residues 213-592 of SEQ ID NO:2. In some embodiments, the TGF-β decoy receptor lacks amino acid sequence corresponding to residues 810-851 of SEQ ID NO:3.

In some embodiments, the TGF-β decoy receptor lacks amino acid sequence corresponding to residues 127-503 of SEQ ID NO:1. In some embodiments, the TGF-β decoy receptor lacks amino acid sequence corresponding to residues 192-592 of SEQ ID NO:2. In some embodiments, the TGF-β decoy receptor lacks amino acid sequence corresponding to residues 788-851 of SEQ ID NO:3.

In some embodiments, the TGF-β decoy receptor may comprise further functional amino acid sequences. For example, the TGF-β decoy receptor may comprise amino acid sequence(s) to facilitate expression, folding, trafficking, processing, purification or detection of the TGF-β decoy receptor. For example, the TGF-β decoy receptor may comprise a sequence encoding a protein tag, e.g. a FLAG, His, (e.g. 6XHis), Myc, GST, MBP, HA, E, or Biotin tag, optionally at the N- or C-terminus.

TGF-β decoy receptors according to the present invention may be detectably labelled or, at least, capable of detection. For example, the TGF-β decoy receptor may be labelled with a radioactive atom or a coloured molecule or a fluorescent molecule or a molecule which can be readily detected in any other way. Suitable detectable molecules include fluorescent proteins, luciferase, enzyme substrates, radiolabels and binding moieties. Labelling may be by conjugation to the TGF-β decoy receptor. The TGF-β decoy receptor may be directly labelled with a detectable label or it may be indirectly labelled. In some embodiments, the label may be selected from: a radio-nucleotide, positron-emitting radionuclide (e.g. for positron emission tomography (PET)), MRI contrast agent or fluorescent label.

TGF-β decoy receptors according to the present invention may be conjugated to a drug moiety, e.g. a cytotoxic small molecule. Such conjugates are useful for the targeted killing of cells expressing the antigen molecule.

### Chimeric Antigen Receptors

The present invention also provides a chimeric antigen receptor (CAR). The CAR comprises a TGF-β binding region as described herein.

Chimeric Antigen Receptors (CARs) are recombinant receptors that provide both antigen-binding and T cell activating functions. CAR structure and engineering is reviewed, for example, in Dotti et al., Immunol Rev (2014) 257(1), hereby incorporated by reference in its entirety.

CARs comprise an antigen-binding region linked to a cell membrane anchor region and a signaling region. An optional hinge region may provide separation between the antigen-binding region and cell membrane anchor region, and may act as a flexible linker.

The antigen-binding region of a CAR may be based on the antigen-binding region of an antibody which is specific for the antigen to which the CAR is targeted, or other agent capable of binding to the target. For example, the antigen-binding domain of a CAR may comprise amino acid sequences for the complementarity-determining regions (CDRs) or complete light chain and heavy chain variable region amino acid sequences of an antibody which binds specifically to the target protein. Antigen-binding domains of CARs may target antigen based on other protein:protein interaction, such as ligand:receptor binding; for example an IL-13Rα2-targeted CAR has been developed using an antigen-binding domain based on IL-13 (see e.g. Kahlon et al. 2004 Cancer Res 64(24): 9160-9166).

In some embodiments, the CAR of the present invention comprises a TGF-β binding region as described herein. In some embodiments, the TGF-β binding region comprises or consists of an amino acid sequence corresponding to the TGF-β binding region of a TGF-β receptor, e.g. the type II TGF-β receptor (TGF-βR2).

The cell membrane anchor region is provided between the antigen-binding region and the signalling region of the CAR. The cell membrane anchor region provides for anchoring the CAR to the cell membrane of a cell expressing a CAR, with the antigen-binding region in the extracellular space, and signalling region inside the cell. Cell membrane anchor regions of CARs have been described which are derived from transmembrane region sequences for CD3-ζ, CD4, CD8 or CD28.

In some embodiments, the CAR of the present invention comprises a cell membrane anchor region as described herein. The cell membrane anchor region may e.g. by a lipid anchor region.

In some embodiments, the CAR of the present invention comprises a TGF-β binding region as described herein and a cell membrane anchor region as described herein. In some embodiments, the CAR comprises or consists of a TGF-β decoy receptor as described herein.

The signalling region allows for activation of the T cell. The CAR signalling regions may comprise the amino acid sequence of the intracellular domain of CD3-ζ, which provides immunoreceptor tyrosine-based activation motifs (ITAMs) for phosphorylation and activation of the CAR-expressing T cell. Signalling regions comprising sequences of other ITAM-containing proteins have also been employed in CARs, such as domains comprising the ITAM containing region of FcγRI (Haynes et al., 2001 J Immunol 166(1):182-187). CARs comprising a signalling region derived from the intracellular domain of CD3-ζ are often referred to as first generation CARs.

Signalling regions of CARs may also comprise co-stimulatory sequences derived from the signalling region of co-stimulatory molecules, to facilitate activation of CAR-expressing T cells upon binding to the target protein. Suitable co-stimulatory molecules include CD28, OX40, 4-1BB, ICOS and CD27. CARs having a signalling region including additional co-stimulatory sequences are often referred to as second generation CARs.

In some cases CARs are engineered to provide for co-stimulation of different intracellular signalling pathways. For example, signalling associated with CD28 costimulation preferentially activates the phosphatidylinositol 3-kinase (P13K) pathway, whereas the 4-1 BB-mediated signalling is through TNF receptor associated factor (TRAF) adaptor proteins. Signalling regions of CARs therefore sometimes contain co-stimulatory sequences derived from signalling regions of more than one co-stimulatory molecule. CARs comprising a signalling region with multiple co-stimulatory sequences are often referred to as third generation CARs.

An optional hinge region may provide separation between the antigen-binding domain and the transmembrane domain, and may act as a flexible linker. Hinge regions may be flexible domains allowing the binding moiety to orient in different directions. Hinge regions may be derived from IgG1 or the CH₂CH₃ region of immunoglobulin.

CARs may be combined with costimulatory ligands, chimeric costimulatory receptors or cytokines to further enhance T cell potency, specificity and safety (Sadelain et al., The basic principles of chimeric antigen receptor (CAR) design. Cancer Discov. 2013 April; 3(4): 388-398. doi:10.1158/2159-8290.CD-12-0548, specifically incorporated herein by reference).

Also provided is a cell comprising a CAR according to the invention. The CAR according to the present invention may be used to generate T cells. Engineering of CARs into T cells may be performed during culture, in vitro, for transduction and expansion, such as happens during expansion of T cells for adoptive T cell therapy.

### Nucleic Acids Encoding the TGF-β Decoy Receptors and CARs

The present invention provides a nucleic acid encoding a TGF-β decoy receptor or CAR according to the present invention. In some embodiments, the nucleic acid is purified or isolated, e.g. from other nucleic acid, or naturally-occurring biological material.

The present invention also provides a vector comprising nucleic acid encoding a TGF-β decoy receptor or CAR according to the present invention.

A “vector” as used herein is a nucleic acid (DNA or RNA) used as a vehicle to transfer exogenous nucleic acid into a cell. The vector may be an expression vector for expression of the nucleic acid in the cell. Such vectors may include a promoter sequence operably linked to the nucleic acid encoding the sequence to be expressed. A vector may also include a termination codon and expression enhancers. Any suitable vectors, promoters, enhancers and termination codons known in the art may be used to express a TGF-β decoy receptor/CAR according to the invention from a vector according to the invention.

In this specification the term “operably linked” may include the situation where a selected nucleic acid sequence and regulatory nucleic acid sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. Where appropriate, the resulting transcript may then be translated into a desired polypeptide.

The nucleic acid and/or vector according to the present invention is preferably provided for introduction into a cell, e.g. a primary human immune cell. Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 or Morgan and Boyerinas, Biomedicines 2016 4, 9, which are both hereby incorporated by reference in its entirety. In some embodiments, the viral vector may be a lentiviral, retroviral, adenoviral, or Herpes Simplex Virus vector. In some embodiments, the lentiviral vector may be pELNS, or may be derived from pELNS. In some embodiments, the vector may be a vector encoding CRISPR/Cas9.

### Cells Comprising/Expressing the TGF-β Decoy Receptors and CARs

The present invention also provides a cell comprising or expressing a TGF-β decoy receptor or CAR according to the present invention. Also provided is a cell comprising or expressing a nucleic acid or vector according to the invention.

The cell may be a eukaryotic cell, e.g. a mammalian cell. The mammal may be a human, or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell may be from, or may have been obtained from, a human subject.

In some embodiments, the cell is a cell which is normally responsive to TGF-β (e.g. TGF-β1, TGF-β2 or TGF-β3), e.g. a cell which expresses a TGF-β receptor (e.g. TGF-βR1, TGF-βR2 or TGF-βR3).

The cell may be an immune cell. The cell may be a cell of hematopoietic origin, e.g. a neutrophil, eosinophil, basophil, dendritic cell, lymphocyte, or monocyte. The lymphocyte may be e.g. a T cell, B cell, NK cell, NKT cell or innate lymphoid cell (ILC), or a precursor thereof. The cell may express e.g. CD3 polypeptides (e.g. CD3γ CD3ϵ CD3ζ or CD3δ), TCR polypeptides (TCRα or TCRβ, CD27, CD28, CD4 or CD8.

In some embodiments, the cell is a T cell. In some embodiments, the T cell is a CD3+ T cell. In some embodiments, the T cell is a CD3+, CD8+ T cell. In some embodiments, the T cell is a cytotoxic T cell (e.g. a cytotoxic T lymphocyte (CTL)).

In some embodiments, the cell may be a cell comprising/expressing a chimeric antigen receptor (CAR), e.g. a CAR-T cell. In some embodiments, the cell may be a cell comprising/expressing a nucleic acid or vector encoding a CAR. Chimeric Antigen Receptors (CARs) are recombinant receptors that provide both antigen-binding and T cell activating functions. CAR structure and engineering is reviewed, for example, in Dotti et al., Immunol Rev (2014) 257(1), hereby incorporated by reference in its entirety.

Also provided is a cell comprising/expressing a CAR, and comprising/expressing a TGF-β decoy receptor or CAR according to the present invention. The cell may be a CAR-T cell. Engineering of CARs into T cells may be performed during culture, in vitro, for transduction and expansion, such as happens during expansion of T cells for adoptive T cell therapy.

In some embodiments, the cell is an antigen-specific T cell. In embodiments herein, an “antigen-specific” T cell is a cell which displays certain functional properties of a T cell in response to the antigen for which the T cell is specific, or a cell expressing said antigen. In some embodiments, the properties are functional properties associated with effector T cells, e.g. cytotoxic T cells.

In some embodiments, an antigen-specific T cell may display one or more of the following properties: cytotoxicity, e.g. to a cell comprising/expressing antigen for which the T cell is specific; proliferation, IFNγ expression, CD107a expression, IL-2 expression, TNFα expression, perforin expression, granzyme expression, granulysin expression, and/or FAS ligand (FASL) expression, e.g. in response to antigen for which the T cell is specific or a cell comprising/expressing antigen for which the T cell is specific.

Herein, “expression” of IFNγ, CD107a, IL-2, TNFα, perforin, granzyme and/or FASL may refer to gene expression or protein expression. Gene expression can be measured by various means known to those skilled in the art, for example by measuring levels of mRNA by quantitative real-time PCR (qRT-PCR), or by reporter-based methods. Similarly, protein expression can be measured by various methods well known in the art, e.g. by antibody-based methods, for example by western blot, immunohistochemistry, immunocytochemistry, flow cytometry, ELISA, ELISPOT, or reporter-based methods. “Increased expression” refers to a level of expression which is greater than the level of expression of the gene/protein by a T cell which has not been contacted with the antigen for which the T cell is specific, or a cell comprising or expressing the antigen for which the T cell is specific, or the level of expression by a T cell in response to a cell not comprising or expressing the antigen for which the T cell is specific.

In some embodiments, the antigen for which the T cell is specific may be a peptide or polypeptide of a virus, e.g. Epstein-Barr virus (EBV), influenza virus, measles virus, hepatitis B virus (HBV), hepatitis C virus (HCV), human immunodeficiency virus (HIV), lymphocytic choriomeningitis virus (LCMV), Herpes simplex virus (HSV) or human papilloma virus (HPV).

The present invention also provides a method for producing a cell comprising a nucleic acid or vector according to the present invention, comprising introducing a nucleic acid or vector according to the present invention into a cell. The present invention also provides a method for producing a cell expressing a TGF-β decoy receptor or CAR according to the present invention, comprising introducing a nucleic acid or vector according to the present invention in a cell. In some embodiments, the methods additionally comprise culturing the cell under conditions suitable for expression of the nucleic acid or vector by the cell. In some embodiments, the methods are performed in vitro.

In some embodiments, introducing an isolated nucleic acid or vector according to the invention into a cell comprises transduction, e.g. retroviral transduction. Accordingly, in some embodiments the isolated nucleic acid or vector is comprised in a viral vector, or the vector is a viral vector. In some embodiments, the method comprises introducing a nucleic acid or vector according to the invention by electroporation, e.g. as described in Koh et al., Molecular Therapy—Nucleic Acids (2013) 2, e114, which is hereby incorporated by reference in its entirety.

The present invention also provides cells obtained or obtainable by the methods for producing a cell according to the present invention.

### Compositions

The present invention also provides compositions comprising a TGF-β decoy receptor, CAR, nucleic acid, vector or cell according to the invention.

TGF-β decoy receptors, CARs, nucleic acids, vectors and cells according to the present invention may be formulated as pharmaceutical compositions for clinical use and may comprise a pharmaceutically acceptable carrier, diluent, excipient or adjuvant.

In accordance with the present invention methods are also provided for the production of pharmaceutically useful compositions, such methods of production may comprise one or more steps selected from: isolating a TGF-β decoy receptor, CAR, cell, nucleic acid or vector as described herein; and/or mixing a TGF-β decoy receptor, CAR, cell, nucleic acid or vector as described herein with a pharmaceutically acceptable carrier, adjuvant, excipient or diluent.

For example, a further aspect of the present invention relates to a method of formulating or producing a medicament or pharmaceutical composition for use in the treatment of a cancer, the method comprising formulating a pharmaceutical composition or medicament by mixing a TGF-β decoy receptor, CAR, cell, nucleic acid or vector as described herein with a pharmaceutically acceptable carrier, adjuvant, excipient or diluent.

### Properties of the TGF-β Decoy Receptors and Cells Expressing the TGF-β Decoy Receptors

The TGF-β decoy receptors and CARs of the present invention and cells expressing such receptors may be characterised by reference to certain properties.

In particular, a TGF-β decoy receptor according to the present invention may possess one or more of the following properties:
Binding to TGF-β (e.g. TGF-β1, TGF-β2 or TGF-β3);
Inhibition of interaction between TGF-β and a TGF-β receptor (e.g. TGF-βR1, TGF-βR2 or TGF-βR3);
Inhibition of signalling mediated by TGF-β;

Inhibition of signalling mediated by binding of TGF-β to a TGF-β receptor.
Attachment to the cell membrane;
Localisation to lipid rafts;
Ability to induce lipid raft aggregation;
Ability to induce lipid associated signalling.

A CAR according the present invention may also be characterised by reference to one or more of such properties.

A TGF-β decoy receptor which is capable of binding to TGF-β preferably binds TGF-β (e.g. TGF-β1, TGF-β2 and/or TGF-β3) with greater affinity, and/or with greater duration than it binds to proteins other than TGF-β. In some embodiments the present TGF-β decoy receptors may bind with greater affinity to TGF-β than to one or more other members of the TGF-β cytokine superfamily. In some embodiments, the present TGF-β decoy receptors may bind with greater affinity to TGF-β (e.g. TGF-β1, TGF-β2 and/or TGF-β3) than to TGF-α.

In some embodiments, the extent of binding to a protein other than TGF-β is less than about 10% of the binding of the TGF-β decoy receptor to TGF-β as measured, e.g., by ELISA, SPR, Bio-Layer Interferometry (BLI), MicroScale Thermophoresis (MST), or by a radioimmunoassay (RIA). Alternatively, the binding specificity for TGF-β may be reflected in terms of binding affinity, where the TGF-β decoy receptor of the present invention binds to TGF-β with a KD that is at least 0.1 order of magnitude (i.e. 0.1×10n, where n is an integer representing the order of magnitude) greater than the KD towards another, non-target molecule. This may optionally be one of at least 0.2, 0.3, 0.4, 0.5, 0.6, 0.7, 0.8, 0.9, 1.0, 1.5, or 2.0.

Binding affinity may be expressed in terms of dissociation constant (KD). Binding affinity can be measured by methods known in the art, such as by ELISA (for example, as described in Antibody Engineering, vol. 1 (2nd Edn), Springer Protocols, Springer (2010), Part V, pp657-665.), Surface Plasmon Resonance (SPR; see e.g. Hearty et al., Methods Mol Biol (2012) 907:411-442; or Rich et al., Anal Biochem. 2008 Feb. 1; 373(1):112-20), Bio-Layer Interferometry (see e.g. Lad et al., (2015) J Biomol Screen 20(4): 498-507; or Concepcion et al., Comb Chem High Throughput Screen. 2009 September; 12(8):791-800), MicroScale Thermophoresis (MST) analysis (see e.g. Jerabek-Willemsen et al., Assay Drug Dev Technol. 2011 August; 9(4): 342-353), or by a radiolabelled antigen binding assay (RIA).

In some embodiments, the TGF-β decoy receptor according to the present invention binds to TGF-β with a KD of 5 μM or less, preferably one of ≤1 μM, ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM ≤3 nM, ≤2 nM, ≤1 nM, 500 pM.

In some embodiments, the TGF-β decoy receptor according to the present invention binds to TGF-β with an affinity of binding (e.g. as determined by ELISA) of EC50=1000 ng/ml or less, preferably one of ≤900 ng/ml, ≤800 ng/ml, ≤700 ng/ml, ≤600 ng/ml, ≤500 ng/ml, ≤400 ng/ml, ≤300 ng/ml, ≤200 ng/ml, ≤100 ng/ml, ≤90 ng/ml, ≤80 ng/ml, ≤70 ng/ml, ≤60 ng/ml, ≤50 ng/ml, ≤40 ng/ml, ≤30 ng/ml, ≤20 ng/ml, ≤15 ng/ml, ≤10 ng/ml, ≤7.5 ng/ml, ≤5 ng/ml, ≤2.5 ng/ml, or ≤1 ng/ml.

The TGF-β decoy receptors according to the present invention inhibit TGF-β mediated signalling. Herein, ‘inhibition’ refers to a reduction, decrease or lessening relative to a control condition. For example, inhibition of a process by a TGF-β decoy receptor refers to a reduction, decrease or lessening of the extent/degree of that process in the absence of the TGF-β decoy receptor, and/or in the presence of an appropriate control receptor.

The skilled person is able to identify an appropriate control condition for a given assay. For example, a control receptor may be a receptor directed against a target protein which is known not to have a role involved in the property being investigated in the assay.

Inhibition may herein also be referred to as neutralisation or antagonism. That is, a TGF-β decoy receptor which is capable of inhibiting a function or process (e.g. interaction, signalling or other activity mediated by TGF-β) may be said to be a ‘neutralising’ or ‘antagonist’ TGF-β decoy receptor with respect to the relevant function or process. For example, a TGF-β decoy receptor which is capable of inhibiting TGF-β mediated signalling may be referred to as a TGF-β decoy receptor which is capable of neutralising TGF-β mediated signalling, or may be referred to as an antagonist of TGF-β mediated signalling.

TGF-β decoy receptors according to the present invention preferably compete for binding to TGF-β with one or more naturally occurring receptors for TGF-β, e.g. TGF-βR1, TGF-βR2, TGF-βR3 or a variant/isoform/homologue or TGF-β binding fragment of TGF-βR1, TGF-βR2 or TGF-βR3. In some embodiments, a TGF-β decoy receptor according to the present invention is a competitive inhibitor of binding of one of more of TGF-βR1, TGF-βR2, TGF-βR3, and variants/isoforms/homologues and TGF-β binding fragments of TGF-βR1, TGF-βR2 and/or TGF-βR3. That is, in some embodiments, the TGF-β decoy receptor is capable of inhibiting interaction between TGF-β and one or more naturally occurring receptors for TGF-β.

As used herein, a ‘naturally occurring receptor’ is a receptor which is found in nature. A naturally occurring receptor and/or the constituent peptides/polypeptides thereof may be the product of transcription, mRNA processing (e.g. splicing), translation, and post-translational processing (e.g. proteolytic cleavage, glycosylation) from endogenous nucleic acid of a given host species.

In some embodiments, the TGF-β decoy receptor according to the present invention binds to TGF-β in the region bound by one or more naturally occurring receptors for TGF-β. In some embodiments, the TGF-β decoy receptor binds to the same region, or overlapping region, of TGF-β as the region bound by one or more naturally occurring receptors for TGF-β. In some embodiments, the naturally occurring receptor for TGF-β is TGF-βR1, TGF-βR2, TGF-βR3. In some embodiments, the naturally occurring receptor for TGF-β is TGF-βR2.

The ability of a TGF-β decoy receptor to compete with a naturally occurring receptor for TGF-β (e.g. TGF-βR1, TGF-βR2, TGF-βR3, or variants/isoforms/homologues or TGF-β binding fragments of TGF-βR1, TGF-βR2 or TGF-βR3) for binding to TGF-β (i.e. inhibit interaction between TGF-β and the naturally occurring receptor), can be determined for example by analysis of interaction between TGF-β and the naturally occurring receptor in the presence of, or following incubation of TGF-β, or TGF-β and the naturally occurring receptor, with the TGF-β decoy receptor, or cells expressing the TGF-β decoy receptor.

An example of a suitable assay to determine whether a given TGF-β decoy receptor competes with TGF-βR1, TGF-βR2, TGF-βR3, or variants/isoforms/homologues or TGF-β binding fragments of TGF-βR1, TGF-βR2 or TGF-βR3 for binding to TGF-β is a competitive binding assay, such as a competition ELISA assay.

A TGF-β decoy receptor which is capable of inhibiting a given interaction (e.g. between TGF-β and one of TGF-βR1, TGF-βR2 or TGF-βR3) is identified by the observation of a reduction/decrease in the level of interaction between the interaction partners in the presence of or following incubation of one or both of the interaction partners with the TGF-β decoy receptor, or cells expressing the TGF-β decoy receptor, as compared to the level of interaction in the absence of the TGF-β decoy receptor or cells expressing the TGF-β decoy receptor (or in the presence of an appropriate control receptor/cells). Suitable analysis can be performed in vitro, e.g. using recombinant interaction partners or using cells expressing the interaction partners. Cells expressing interaction partners may do so endogenously, or may do so from nucleic acid introduced into the cell. For the purposes of such assays, one or both of the interaction partners and/or the TGF-β decoy receptor may be labelled or used in conjunction with a detectable entity for the purposes of detecting and/or measuring the level of interaction.

Such assays are also useful to determine whether a TGF-β decoy receptor binds to the same region or overlapping region of TGF-β as the region bound by one or more naturally occurring receptors for TGF-β. That is, observation of a reduction/decrease in the level of interaction between TGF-β (e.g. TGF-β1, TGF-β2 or TGF-β3) and e.g. TGF-βR1, TGF-βR2 or TGF-βR3 in the presence of or following incubation of one or both of the interaction partners with the TGF-β decoy receptor or cells expressing the TGF-β decoy receptor, as compared to the level of interaction in the absence of the TGF-β decoy receptor/cells suggests that TGF-β decoy receptor binds to the same region or overlapping region of TGF-β as the region bound by one or more naturally occurring receptors for TGF-β.

Whether a TGF-β decoy receptor according to the present invention binds to TGF-β in the same or same region or overlapping region of TGF-β as the region bound by a naturally occurring receptor for TGF-β can also be determined by analysis of interaction using various methods well known in the art, including X-ray co-crystallography analysis of receptor-ligand complexes, peptide scanning, mutagenesis mapping, hydrogen-deuterium exchange analysis by mass spectrometry, phage display and proteolysis-based ‘protection’ methods. Such methods are described, for example, in Gershoni et al., BioDrugs, 2007, 21(3):145-156, incorporated by reference hereinabove.

Ability of a TGF-β decoy receptor to inhibit interaction between two interaction partners can also be determined by analysis of the downstream functional consequences of the interaction, e.g. TGF-β receptor signalling.

Assays for TGF-β signalling are known in the art, and include e.g. gene expression assays for genes whose transcription is upregulated/downregulated in response to stimulation with TGF-β, assays analysing activation (e.g. as determined by analysis of phosphorylation of relevant factors) of intracellular mediators of TGF-β signalling, assays for analysing changes in expression/processing of proteins associated TGF-β signalling, and assays for analysing phenotypic changes in cells associated with TGF-β signalling.

For example, assays of TGF-β signalling may involve treating cells with TGF-β and analysing phosphorylation of one or more of SMAD2, SMAD3, SMAD1, SMADS, SMAD9 and p38, and/or analysing expression of one or more of CREB and NFκB. Assays of TGF-β signalling can be performed e.g. as described in Example 8 of the present disclosure.

Such assays are also useful for analysis of signalling mediated by TGF-β, e.g. signalling mediated by binding of TGF-β (e.g. TGF-β1, TGF-β2 or TGF-β3) to a TGF-β receptor (e.g. TGF-βR1, TGF-βR2 or TGF-βR3).

As used herein, ‘TGF-β mediated signalling’ and/or processes mediated by TGF-β include signalling mediated by fragments of TGF-β and polypeptide complexes comprising TGF-β or fragments thereof. TGF-β mediated signalling may be signalling mediated by human TGF-β and/or mouse TGF-β. Signalling mediated by TGF-β may occur following binding of TGF-β or a TGF-β containing complex to a receptor to which TGF-β or said complex binds.

Gene expression can be measured by various means known to those skilled in the art, for example by measuring levels of mRNA by quantitative real-time PCR (qRT-PCR), or by reporter-based methods. Similarly, protein expression can be measured by various methods well known in the art, e.g. by antibody-based methods, for example by western blot, immunohistochemistry, immunocytochemistry, flow cytometry, ELISA, ELISPOT, or reporter-based methods.

In some embodiments, the TGF-β decoy receptor according to the present invention, or cells expressing the receptor, are capable of inhibiting interaction between TGF-β (e.g. TGF-β1, TGF-β2 or TGF-β3) and TGF-β receptor (e.g. TGF-βR1, TGF-βR2 or TGF-βR3) to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between the TGF-β and TGF-β receptor in the absence of the TGF-β decoy receptor or cells expressing the TGF-β decoy receptor (or in the presence of an appropriate control receptor/cells). In some embodiments, the TGF-β decoy receptor, or cells expressing the receptor, are capable of inhibiting interaction between the TGF-β and TGF-β receptor to less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between the TGF-β and TGF-β receptor in the absence of the TGF-β decoy receptor or cells expressing the TGF-β decoy receptor (or in the presence of an appropriate control receptor/cells).

In some embodiments, TGF-β decoy receptors according to the present invention are capable of inhibiting the biological activity of TGF-β. In some embodiments, the TGF-β decoy receptor binds to TGF-β in a region which is important for binding to a receptor for TGF-β (e.g. TGF-βR1, TGF-βR2 or TGF-βR3), and thereby disrupts binding to and/or signalling through the receptor.

In some embodiments, the TGF-β decoy receptor according to the present invention is an antagonist of one or more signalling pathways which are activated by signal transduction through a receptor for TGF-β, e.g. TGF-βR1, TGF-βR2 or TGF-βR3. In some embodiments, the TGF-β decoy receptor is capable of inhibiting signalling through one or more immune receptor complexes comprising a TGF-β receptor.

In some embodiments, the TGF-β decoy receptor according to the present invention, or cells expressing the receptor, are capable of inhibiting TGF-β mediated signalling to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of signalling in the absence of the TGF-β decoy receptor or cells expressing the TGF-β decoy receptor (or in the presence of an appropriate control receptor/cells). In some embodiments, the TGF-β decoy receptor, or cells expressing the receptor, are capable of inhibiting TGF-β mediated signalling to less than 1 times, e.g. one of ≤0.99 times, ≤0.95 times, ≤0.9 times, ≤0.85 times, ≤0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of signalling in the absence of the TGF-β decoy receptor or cells expressing the TGF-β decoy receptor (or in the presence of an appropriate control receptor/cells).

Such signalling can be analysed e.g. by treating cells expressing TGF-β receptor (e.g. TGF-βR1, TGF-βR2 or TGF-βR3) and with TGF-β.

The TGF-β decoy receptor according to the present invention may be attached to the cell membrane in cells expressing the TGF-β decoy receptor, e.g. through a cell membrane anchor region. In some embodiments, the cell membrane anchor region may be a lipid anchor region, which may comprise or consist of a lipid anchor, e.g. a GPI anchor.

In some embodiments, the TGF-β decoy receptor may attach to the cell membrane of cells expressing the TGF-β decoy receptor, through association (e.g. covalent association) of the receptor with the cell membrane or a component thereof (e.g. phospholipid). In some embodiments, the TGF-β decoy receptor may attach to the outer layer of the cell membrane bilayer. In some embodiments, the TGF-β decoy receptor may attach to the cell membrane of cells expressing the TGF-β decoy receptor, through association of a GPI anchor with the cell membrane or a component thereof. In some embodiments, the TGF-β decoy receptor may attach to the cell membrane of cells expressing the TGF-β decoy receptor, through interaction between lipid component of the GPI anchor with lipid component of the cell membrane.

Release of GPI anchored proteins can be achieved through treatment with phospholipase C (PLC), which hydrolyzes the phosphodiester bond of phosphatidylinositol.

In some embodiments, the TGF-β decoy receptor may localise to lipid rafts within the cell membrane of cells expressing the TGF-β decoy receptor. Localisation to lipid rafts may be through the lipid anchor region (e.g. GPI anchor).

Lipid rafts are areas of the cell membrane characterised by being enriched in cholesterol, glycosphingolipids, sphingomyelin, phospholipids with acyl chains, and GPI-linked proteins, as well as other membrane proteins such as innate immune receptors (Lingwood and Simmons 2010, Science 327:46-50).

Localisation of a given factor to lipid rafts can be analysed by means well known in the art. For example, lipid rafts of live cells can be labelled e.g. with a fluorescent label, localisation of the factor to the lipid rafts can be analysed and quantified. One example of a lipid raft labelling kit is the Vybrant® Alexa Fluor® 555 Lipid Raft Labeling Kit (ThermoFisher Scientfic). Lipid raft localisation of a given factor can also by analysed by isolation of the lipid raft component from a cell expressing the factor, and subsequent analysis of the lipid raft for the factor (e.g. by antibody-based methods, for example by western blot, ELISA, immunostaining, etc. or reporter based methods). Isolation and analysis of lipid rafts is described, for example, in Lee, Methods Mol Biol. 2013;1066:131-45 and Lemaire-Vieille et al. (2013) Bio-protocol 3(16): e854, both of which are hereby incorporated by reference in their entirety.

In some embodiments, the TGF-β decoy receptor of the present invention may induce lipid raft aggregation in cells expressing the receptor. In some embodiments, the TGF-β decoy receptor of the present invention may induce lipid associated signalling.

Lipid raft aggregation can be analysed e.g. as described in Janes et al. 1999, The Journal of Cell Biology, 147(2): 447-461. Lipid associated signalling may be signalling mediated by a lipid signalling molecule, e.g. sphingolipid (ceramide, sphingosine, sphingosine-1-phosphate, glucosylceramide, ceramide-1-phosphate, phosphatidylinositol bisphosphate, lysophosphatidic acid (LPA), platelet activating factor (PAF), endocannabinoids, prostaglandins, fatty acid esters of hydroxy fatty acids (FAHFA), retinol derivatives, steroid hormones, retinoic acid or prostaglandins. Lipid associated signalling may involve downstream activation e.g. of src, ras or rac. Lipid associated signalling can be analysed e.g. using gene expression assays for genes whose transcription is upregulated/downregulated by lipid associated signalling, assays analysing activation (e.g. as determined by analysis of phosphorylation of relevant factors) of intracellular mediators of lipid associated signalling, assays for analysing changes in expression/processing of proteins associated lipid associated signalling, and assays for analysing phenotypic changes in cells associated with lipid associated signalling.

Lipid raft aggregation and lipid associated signalling may provide for enhanced T cell receptor signalling, and may also be able to reverse the effect of the TGF-β mediated signalling. Lipid raft aggregation and lipid associated signalling may lead to downstream activation of one or more of src, ras, rac or sox, and so the TGF-β decoy receptors of the present invention may be capable of converting the inhibitory signal from TGF-β into a signal for T cell activation. In this way, the effect of TGF-β treatment on cells comprising/expressing TGF-β decoy receptors according to the present invention may be stimulatory, rather than inhibitory.

In some embodiments, the TGF-β decoy receptor of the present invention may localise to lipid rafts of cells expressing the TGF-β decoy receptor to a greater extent than another TGF-β binding protein. In some embodiments, the TGF-β decoy receptor of the present invention localises to lipid rafts of cells expressing the TGF-β decoy receptor to a greater extent than a naturally occurring TGF-β binding protein such as TGF-β receptor (e.g. TGF-βR1, TGF-βR2 or TGF-βR3), BAMBI (as described in Onichtchouk et al. supra), or a TGF-β binding protein described in Bollard et al., Russo et al., Zhang et al. or Penafuerte et al. (supra), such as DN-TGF-βR2.

The extent of localisation of a given molecule to lipid rafts can be determined e.g. by analysing lipid raft and non-lipid raft fractions of cells expressing the relevant molecule, and determining the proportion of the molecule associated with the lipid raft and/or non-lipid raft fractions.

In some embodiments, the TGF-β decoy receptor of the present invention may induce lipid raft aggregation in cells expressing the receptor to a greater extent as compared to another TGF-β binding protein. In some embodiments, the TGF-β decoy receptor of the present invention may induce lipid associated signalling to a greater extent as compared to another TGF-β binding protein.

Herein, localisation to lipid rafts, ability to induce lipid raft aggregation, ability to induce lipid associated signalling to a ‘greater extent’ may be greater than 1 times, e.g. ≥1.01 times, ≥1.02 times, ≥1.03 times, ≥1.04 times, ≥1.05 times, ≥1.06 times, ≥1.07 times, ≥1.08 times, ≥1.09 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥2.1 times, ≥2.2 times, ≥2.3 times, ≥2.4 times, ≥2.5 times, ≥2.6 times, ≥2.7 times, ≥2.8 times, ≥2.9 times, ≥3 times, ≥3.5 times, ≥4times, ≥4.5 times, ≥5 times, ≥6 times, ≥7 times, ≥8 times, ≥9 times, ≥10 times, ≥15 times, ≥20 times, ≥25 times, ≥30 times, ≥35 times, ≥40 times, ≥45 times, ≥50 times, ≥60 times, ≥70 times, ≥80 times, ≥90 times, ≥100 times, ≥200 times, ≥300 times, ≥400 times, ≥500 times, ≥600 times, ≥700 times, ≥800 times, ≥900 times, ≥1000 times the extent of localisation, induction of lipid raft aggregation or induction of lipid associated signalling displayed by another TGF-β binding protein, e.g. TGF-β receptor (e.g. TGF-βR1, TGF-βR2 or TGF-βR3), BAMBI (as described in Onichtchouk et al. supra), or a TGF-β binding protein described in Bollard et al., Russo et al., Zhang et al. or Penafuerte et al. (supra), such as DN-TGF-βR2, in cells expressing said TGF-β binding protein.

In some embodiments, the TGF-β decoy receptor of the present invention may display an increased level of expression at the surface of cells expressing the TGF-β decoy receptor as compared to the level of expression of another TGF-β binding protein at the surface of cells expressing said TGF-β binding protein. For example, the TGF-β decoy receptor may exhibit improved trafficking to the cell surface, may be more stably associated with the cell membrane, may exhibit reduced internalisation from the cell surface, or may exhibit reduced release from the cell surface.

The increased level of surface expression of the TGF-β decoy receptor is associated with the advantage that the amount of TGF-β available for binding to signalling-competent TGF-β receptor is reduced, resulting in greater inhibition of TGF-β mediated signalling.

In some embodiments, the TGF-β decoy receptor of the present invention may display an increased level of surface expression on cells expressing the TGF-β decoy receptor as compared to the level of surface expression of a naturally occurring TGF-β binding protein such as TGF-β receptor (e.g. TGF-βR1, TGF-βR2 or TGF-βR3), BAMBI (as described in Onichtchouk et al. supra), or a TGF-β binding protein described in

Bollard et al., Russo et al., Zhang et al. or Penafuerte et al. (supra), such as DN-TGF-βR2, on cells expressing such TGF-β binding protein. Cell surface expression of a given protein can be measured by various methods well known in the art, e.g. by antibody-based methods, for example by western blot, immunohistochemistry, immunocytochemistry, flow cytometry, ELISA, ELISPOT, or reporter-based methods.

For example, assays of cell surface expression of a TGF-β decoy receptor may involve analysis using an antibody capable of recognising the TGF-β binding region of the decoy receptor. Assays can be performed e.g. as described in Example 7 of the present disclosure.

An increased level of cell surface expression may be greater than 1 times, e.g. ≥1.01 times, ≥1.02 times, ≥1.03 times, ≥1.04 times, ≥1.05 times, ≥1.06 times, ≥1.07 times, ≥1.08 times, ≥1.09 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥2.1 times, ≥2.2 times, ≥2.3 times, ≥2.4 times, ≥2.5 times, ≥2.6 times, ≥2.7 times, ≥2.8 times, ≥2.9 times, ≥3 times, ≥3.5 times, ≥4 times, ≥4.5 times, ≥5 times, ≥5 times, ≥6 times, ≥7 times, ≥8 times, ≥9 times, ≥10 times, ≥15 times, ≥20 times, ≥25 times, ≥30 times, ≥35 times, ≥40 times, ≥45 times, ≥50 times, ≥60 times, ≥70 times, ≥80 times, ≥90 times, ≥100 times, ≥200 times, ≥300 times, ≥400 times, ≥500 times, ≥600 times, ≥700 times, ≥800 times, ≥900 times, ≥1000 times the level of surface expression displayed by another TGF-β binding protein, e.g. TGF-β receptor (e.g. TGF-βR1, TGF-βR2 or TGF-βR3), BAMBI (as described in Onichtchouk et al. supra), or a TGF-β binding protein described in Bollard et al., Russo et al., Zhang et al. or Penafuerte et al. (supra), such as DN-TGF-βR2, in cells expressing said TGF-β binding protein.

In some embodiments, the TGF-β decoy receptor of the present invention may be capable of inhibiting TGF-β mediated signalling in cells expressing the TGF-β decoy receptor to a greater extent than another TGF-β binding protein. For example, the TGF-β decoy receptor may exhibit improved (e.g. greater affinity and/or greater avidity) binding to TGF-β (e.g. TGF-β1, TGF-β32 or TGF-β3), or improved ability to inhibit interaction of TGF-β with signalling-competent TGF-β receptor (e.g. TGF-βR1, TGF-βR2 or TGF-βR3). TGF-β mediated signalling by cells expressing a given TGF-β binding protein can be analysed as described herein, e.g. following treatment of the cells with TGF-β.

Inhibition of TGF-β mediated signalling to a ‘greater extent’ may be greater than 1 times, e.g. ≥1.01 times, ≥1.02 times, ≥1.03 times, ≥1.04 times, ≥1.05 times, ≥1.06 times, ≥1.07 times, ≥1.08 times, ≥1.09 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥2.1 times, ≥2.2 times, ≥2.3 times, ≥2.4 times, ≥2.5 times, ≥2.6 times, ≥2.7 times, ≥2.8 times, ≥2.9 times, ≥3 times, ≥3.5 times, ≥4 times, ≥4.5 times, ≥5 times, ≥6 times, ≥7 times, ≥8 times, ≥9 times, ≥10 times, ≥15 times, ≥20 times, ≥25 times, ≥30 times, ≥35 times, ≥40 times, ≥45 times, ≥50 times, ≥60 times, ≥70 times, ≥80 times, ≥90 times, ≥100 times, ≥200 times, ≥300 times, ≥400 times, ≥500 times, ≥600 times, ≥700 times, ≥800 times, ≥900 times, ≥1000 times the extent inhibition of TGF-β mediated signalling displayed by another TGF-β binding protein, e.g. TGF-β receptor (e.g. TGF-βR1, TGF-βR2 or TGF-βR3), BAMBI (as described in Onichtchouk et al. supra), or a TGF-β binding protein described in Bollard et al., Russo et al., Zhang et al. or Penafuerte et al. (supra), such as DN-TGF-βR2.

In some embodiments, the TGF-β decoy receptor of the present invention may be secreted from cells expressing the decoy receptor to a greater extent than the level of secretion of another TGF-β binding protein from cells expressing that TGF-β binding protein. Advantageously, secreted TGF-β decoy receptors are capable of binding to soluble TGF-β, thereby reducing the level of soluble TGF-β available to engage cell signalling-competent TGF-β receptors.

The level of secretion of a given protein from a cell can be analysed by measuring the amount of the receptor in cell culture supernatant of cells expressing the protein, e.g. by antibody-based methods, for example by western blot, immunohistochemistry, immunocytochemistry, flow cytometry, ELISA, ELISPOT, or reporter-based methods.

Secretion to a ‘greater extent’ may be greater than 1 times, e.g. ≥1.01 times, ≥1.02 times, ≥1.03 times, ≥1.04 times, ≥1.05 times, ≥1.06 times, ≥1.07 times, ≥1.08 times, ≥1.09 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥2.1 times, ≥2.2 times, ≥2.3 times, ≥2.4 times, ≥2.5 times, ≥2.6 times, ≥2.7 times, ≥2.8 times, ≥2.9 times, ≥3 times, ≥3.5 times, ≥4 times, ≥4.5 times, ≥5 times, ≥6 times, ≥7 times, ≥8 times, ≥9 times, ≥10 times, ≥15 times, ≥20 times, ≥25 times, ≥30 times, ≥35 times, ≥40 times, ≥45 times, ≥50 times, ≥60 times, ≥70 times, ≥80 times, ≥90 times, ≥100 times, ≥200 times, ≥300 times, ≥400 times, ≥500 times, ≥600 times, ≥700 times, ≥800 times, ≥900 times, ≥1000 times the extent of secretion of another TGF-β binding protein, e.g. TGF-β receptor (e.g. TGF-βR1, TGF-βR2 or TGF-βR3), BAMBI (as described in Onichtchouk et al. supra), or a TGF-β binding protein described in Bollard et al., Russo et al., Zhang et al. or Penafuerte et al. (supra), such as DN-TGF-βR2, from cells expressing said TGF-β binding protein.

Cells expressing a TGF-β decoy receptor according to the present invention may display a reduced level of TGF-β mediated signalling, e.g. following treatment with TGF-β, as compared to the level of TGF-β mediated signalling by a comparable cell not expressing the TGF-β decoy receptor. TGF-β mediated signalling by cells can be analysed as described herein.

In some embodiments, cells expressing a TGF-β decoy receptor according to the present invention display a level of TGF-β mediated signalling (e.g. in response to treatment with TGF-β) which is less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of signalling by comparable cells not expressing the TGF-β decoy receptor. In some embodiments, cells expressing a TGF-β decoy receptor according to the present invention display a level of TGF-β mediated signalling (e.g. in response to treatment with TGF-β) which is less than 1 times, e.g. one of ≤0.99 times, ≤0.95 times, ≤0.9 times, ≤0.85 times, ≥0.8 times, ≤0.85 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of signalling by comparable cells not expressing the TGF-β decoy receptor.

Similarly, cells expressing a TGF-β decoy receptor according to the present invention may exhibit a reduced level of a response associated with TGF-β mediated signalling, e.g. following treatment with TGF-β, as compared to the level of the response by a comparable cell not expressing the TGF-β decoy receptor. That is, a cell expressing a TGF-β decoy receptor according to the present invention may display reduced sensitivity to treatment with TGF-β as compared to a comparable cell not expressing the TGF-β decoy receptor.

In some embodiments, the cells expressing a TGF-β decoy receptor may exhibit reduced sensitivity to TGF-β mediated suppression of an activity of the cell. A cell expressing the TGF-β decoy receptor of the present invention may display one or more of the following properties as compared to a comparable cell not expressing the TGF-β decoy receptor, e.g. following treatment with TGF-β:
Increased rate of proliferation;
Increased expression of one or more growth factors (e.g. IL-2);
Increased survival;
Increased expression of one or more cytotoxic/effector factors (e.g. IFNβ, granzyme, perforin, granulysin, CD107a, TNFα, FASL);
Increased cytotoxicity;
Improved anticancer effect.

In some embodiments, cells expressing a TGF-β decoy receptor according to the present invention may exhibit one or more of the properties described in the preceding paragraph following treatment with TGF-β as compared to the level of the property by the same cells in the absence of treatment with TGF-β.

The cell may be a cell of hematopoietic origin, e.g. a neutrophil, eosinophil, basophil, dendritic cell, lymphocyte, or monocyte. The lymphocyte may be e.g. a T cell, B cell, NK cell, NKT cell or innate lymphoid cell (ILC), or a precursor thereof. The cell may express e.g. CD3 polypeptides (e.g. CD3γ CD3ϵ CD3ζ or CD3δ), TCR polypeptides (TCRα or TCRβ), CD27, CD28, CD4 or CD8. In some embodiments, the cell may comprise or express a CAR (or may comprise/express nucleic acid/vector encoding a CAR); e.g. the cell may be a CAR-T cell.

These properties can be analyzed by methods well known to the skilled person.

The rate of proliferation or expansion of a cell or population of cells can be analysed e.g. by measuring the number of cells at different time points, or by analysis of incorporation of ³H-thymidine or CFSE dilution assay, e.g. as described in Fulcher and Wong, Immunol Cell Biol (1999) 77(6): 559-564. Proliferation and/or expansion may be measured in vivo, or in culture in vitro or ex vivo.

Gene or protein expression of growth factors and cytotoxic/effector factors can be measured e.g. by qPCR analysis of mRNA levels, and/or by immunoassay based methods for detecting the relevant protein, such as ELISA, flow cytometry, immunoblot, etc.

Survival/persistence of cells (e.g. survival/persistence in vivo, or in culture in vitro or ex vivo) may be determined by monitoring cell number over time, by detecting cells labelled with a detectable label.

Cytotoxicity can be investigated, for example, using any of the methods reviewed in Zaritskaya et al., Expert Rev Vaccines (2011), 9(6):601-616, hereby incorporated by reference in its entirety, e.g. by ⁵¹Cr release assay.

An anti-cancer effect may be investigated e.g. by analysing the ability of a cell expressing a TGF-β decoy receptor to kill cells of a cancer, e.g. in vitro. The cancer may express an antigen for which the cell is specific. An anti-cancer effect may also be investigated in vivo in an animal model of a cancer, e.g. by analysing development, progression or severity of symptoms of a cancer, and/or survival of the animals, following administration of cells expressing a TGF-β decoy receptor according to the invention, and comparison to an appropriate control condition.

Increased gene or protein expression, proliferation, expansion, survival, cytotoxicity or anti-cancer effect for a cell expressing a TGF-β decoy receptor according to the present invention may be one of greater than 1 times, e.g. ≥≥1.01 times, ≥1.02 times, ≥1.03 times, ≥1.04 times, ≥1.05 times, ≥1.06 times, ≥1.07 times, ≥1.08 times, ≥1.09 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥2.1 times, ≥2.2 times, ≥2.3 times, ≥2.4 times, ≥2.5 times, ≥2.6 times, ≥2.7 times, ≥2.8 times, ≥2.9 times, ≥3 times, ≥3.5 times, ≥4 times, ≥4.5 times, ≥5 times, ≥6 times, ≥7 times, ≥8 times, ≥9 times, ≥10 times, ≥15 times, ≥20 times, ≥25 times, ≥30 times, ≥35 times, ≥40 times, ≥45 times, ≥50 times, ≥60 times, ≥70 times, ≥80 times, ≥90 times, ≥100 times, ≥200 times, ≥300 times, ≥400 times, ≥500 times, ≥600 times, ≥700 times, ≥800 times, ≥900 times, ≥1000 times the level of expression, proliferation, expansion, survival, cytotoxicity or anti-cancer effect displayed by a comparable cell not expressing the TGF-β decoy receptor.

In some embodiments, cells expressing a TGF-β decoy receptor according to the present invention may exhibit reduced sensitivity to TGF-β mediated suppression of an activity of the cell as compared to a comparable cell expressing another TGF-β binding protein, e.g. TGF-β receptor (e.g. TGF-βR1, TGF-βR2 or TGF-βR3), BAMBI (as described in Onichtchouk et al. supra), or a TGF-β binding protein described in Bollard et al., Russo et al., Zhang et al. or Penafuerte et al. (supra), such as DN-TGF-βR2.

In some embodiments, a cell expressing a TGF-β decoy receptor according to the present invention may exhibit one or more of the following properties as compared to a comparable cell expressing another TGF-β binding protein, e.g. TGF-β receptor (e.g. TGF-βR1, TGF-βR2 or TGF-βR3), BAMBI (as described in Onichtchouk et al. supra), or a TGF-β binding protein described in Bollard et al., Russo et al., Zhang et al. or Penafuerte et al. (supra), such as DN-TGF-βR2, e.g. following treatment with TGF-β:
Increased rate of proliferation or expansion, e.g. in vitro and/or in vivo;
Increased expression of one or more growth factors (e.g IL-2);
Increased survival;
Increased expression of one or more cytotoxic/effector factors (e.g. IFNγ, granzyme, perforin, granulysin, CD107a, TNFα, FASL);
Increased cytotoxicity;
Improved anticancer effect.

Increased gene or protein expression, proliferation, expansion, survival, cytotoxicity or anti-cancer effect for a cell expressing a TGF-β decoy receptor according to the present invention may be one of greater than 1 times, e.g. ≥1.01 times, ≥1.02 times, ≥1.03 times, ≥1.04 times, ≥1.05 times, ≥1.06 times, ≥1.07 times, ≥1.08 times, ≥1.09 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥2.1 times, ≥2.2 times, ≥2.3 times, ≥2.4 times, ≥2.5 times, ≥2.6 times, ≥2.7 times, ≥2.8 times, ≥2.9 times, ≥3 times, ≥3.5 times, ≥4 times, ≥4.5 times, ≥5 times, ≥6 times, ≥7 times, ≥8 times, ≥9 times, ≥10 times, ≥15 times, ≥20 times, ≥25 times, ≥30 times, ≥35 times, ≥40 times, ≥45 times, ≥50 times, ≥60 times, ≥70 times, ≥80 times, ≥90 times, ≥100 times, ≥200 times, ≥300 times, ≥400 times, ≥500 times, ≥600 times, ≥700 times, ≥800 times, ≥900 times, ≥1000 times the level of expression, proliferation, expansion, survival, cytotoxicity or anti-cancer effect displayed by a comparable cell expressing another TGF-β binding protein, e.g. TGF-β receptor (e.g. TGF-βR1, TGF-βR2 or TGF-βR3), BAMBI (as described in Onichtchouk et al. supra), or a TGF-β binding protein described in Bollard et al., Russo et al., Zhang et al. or Penafuerte et al. (supra), such as DN-TGF-βR2.

### Therapeutic Applications

The TGF-β decoy receptors, CARs, nucleic acids, vectors, cells and pharmaceutical compositions according to the present invention find use in therapeutic and prophylactic methods.

The present invention provides a TGF-β decoy receptor, CAR, nucleic acid, vector, cell or pharmaceutical composition according to the present invention for use in a method of medical treatment or prophylaxis. The present invention also provides the use of a TGF-β decoy receptor, CAR, nucleic acid, vector, cell or pharmaceutical composition according to the present invention in the manufacture of a medicament for treating or preventing a disease or condition. The present invention also provides a method of treating or preventing a disease or condition, comprising administering to a subject a therapeutically or prophylactically effective amount of a TGF-β decoy receptor, CAR, nucleic acid, vector, cell or pharmaceutical composition according to the present invention.

In particular, the TGF-β decoy receptors, CARs, nucleic acids, vectors, cells and pharmaceutical compositions according to the present invention find use to treat or prevent diseases/conditions associated with TGF-β mediated signalling. As explained herein, the TGF-β decoy receptors, CARs, nucleic acids, cells and compositions of the present invention find use to reduce the level of TGF-β mediated signalling. Accordingly, the TGF-β decoy receptors, CARs, nucleic acids, vectors, cells and compositions of the present invention are useful for the treatment or prevention of diseases/conditions for which a reduction in the level of TGF-β mediated signalling would provide a therapeutic or prophylactic benefit.

‘Treatment’ may, for example, be reduction in the development or progression of a disease/condition, alleviation of the symptoms of a disease/condition or reduction in the pathology of a disease/condition. Treatment or alleviation of a disease/condition may be effective to prevent progression of the disease/condition, e.g. to prevent worsening of the condition or to slow the rate of development. In some embodiments treatment or alleviation may lead to an improvement in the disease/condition, e.g. a reduction in the symptoms of the disease/condition or reduction in some other correlate of the severity/activity of the disease/condition. Prevention of a disease/condition may refer to prevention of a worsening of the condition or prevention of the development of the disease/condition, e.g. preventing an early stage disease/condition developing to a later, chronic, stage.

In some embodiments, the disease or condition to be treated or prevented may be a disease/condition associated with TGF-β mediated signalling (e.g. a disease/condition in which TGF-β mediated signalling is pathologically implicated), and/or for which TGF-β mediated signalling is a risk factor. In some embodiments, the disease/condition may be associated with an increased level of TGF-β mediated signalling as compared to the control state.

The treatment may be aimed at reducing the level of TGF-β mediated signalling, and/or reducing the level of a response associated with TGF-β mediated signalling.

In some embodiments, the treatment may be aimed at reducing the level of TGF-β mediated signalling. Administration of the TGF-β decoy receptors, CARs, nucleic acids, vectors, cells and compositions of the present invention may cause a reduction in the level of TGF-β mediated signalling through sequestration of TGF-β.

In some embodiments, the treatment may be aimed at reducing the level of TGF-β mediated signalling in a subject, or a cell, population of cells, tissue or organ of a subject.

In some embodiments, the treatment may comprise modifying a cell or population of cells to comprise/express a TGF-β decoy receptor, CAR, nucleic acid or vector of the present invention, which may then be less responsive to TGF-β. In some embodiments, the treatment may comprise administering to a subject a cell or population of cells modified to comprise/express a TGF-β decoy receptors, CARs, nucleic acids or vectors of the present invention.

In some embodiments, the treatment is aimed at providing the subject with an immune cell or population of immune cells which are less sensitive to TGF-β mediated inhibition of an activity of the cell/population, e.g. by administering a cell according to the present invention, or generating a cell according to the present invention.

The subject to be treated may be any animal or human. The subject is preferably mammalian, more preferably human. The subject may be a non-human mammal, but is more preferably human. The subject may be male or female. The subject may be a patient. A subject may have been diagnosed with a disease or condition requiring treatment, or be suspected of having such a disease or condition.

The subject to be treated may display an elevated level of TGF-β mediated signalling, e.g. as determined by analysis of the subject, or a sample (e.g. a cell, tissue, blood sample) obtained from the subject, using an appropriate assay.

The subject may have an increased level of expression or activity of a positive regulator/effector of TGF-β mediated signalling such as TGF-β (e.g. TGF-β1, TGF-β2 and/or TGF-β3) or a TGF-β receptor (e.g. TGF-βR1, TGF-↑R2 and/or TGF-βR3), or may have an increased level of expression or activity of a factor upregulated by TGF-β mediated signalling. The subject may have an increased level of an activity upregulated by TGF-β mediated signalling.

The subject may have a reduced level of expression or activity of a negative regulator of TGF-β mediated signalling, or may have a reduced level of expression or activity of a factor downregulated by TGF-β mediated signalling. The subject may have a reduced level of an activity downregulated by TGF-β mediated signalling.

The increase/reduction may be relative to the level of expression/activity in the absence of the relevant disease/condition, e.g. the level of expression/activity in a healthy control subject or sample obtained from a healthy control subject.

In some embodiments the subject may be at risk of developing/contracting a disease or condition.

In some embodiments, the disease or condition to be treated or prevented may be a cancer. TGF-β mediated signalling is implicated in the development/progression of a variety of cancers. TGFβ signalling in cancer is reviewed, for example in Nacif and Shaker 2014, Akhurst and Hata 2012, and Meulmeester and ten Dijke 2011 (supra).

The cancer may be any unwanted cell proliferation (or any disease manifesting itself by unwanted cell proliferation), neoplasm or tumor or increased risk of or predisposition to the unwanted cell proliferation, neoplasm or tumor. The cancer may be benign or malignant and may be primary or secondary (metastatic). A neoplasm or tumor may be any abnormal growth or proliferation of cells and may be located in any tissue. Examples of tissues include the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, haematologic cancer and sarcoma.

In some embodiments, the cancer to be treated is one or more of nasopharyngeal carcinoma (NPC; e.g. Epstein-Barr Virus (EBV)-positive NPC), cervical carcinoma (CC; e.g. human papillomavirus (HPV)-positive CC), oropharyngeal carcinoma (OPC; e.g. HPV-positive OPC), gastric carcinoma (GC; e.g. EBV-positive GC), hepatocellular carcinoma (HCC; e.g. Hepatitis B Virus (HBV)-positive HCC), lung cancer (e.g. non-small cell lung cancer (NSCLC)) and head and neck cancer (e.g. cancer originating from tissues of the lip, mouth, nose, sinuses, pharynx or larynx, e.g. head and neck squamous cell carcinoma (HNSCC)).

In some embodiments the cancer is associated with, or caused by, a virus. In some embodiments the cancer is an EBV-positive cancer. In some embodiments the cancer is an HPV-positive cancer.

In some embodiments, the cancer is one of a head and neck cancer, nasopharyngeal carcinoma (NPC), oropharyngeal cancer (OPC), cervical cancer (CC), gastric/stomach cancer, gastric carcinoma or lung cancer.

In some embodiments the cancer is a cancer expressing TGF-β (e.g. TGF-β1, TGF-β2 and/or TGF-β3) or a TGF-β receptor (e.g. TGF-βR1, TGF-βR2 and/or TGF-βR3). A cancer may be determined to express TGF-β or a TGF-β receptor by any suitable means, which are well known to the skilled person. A cancer expressing TGF-β or a TGF-β receptor may be identified by detection of expression of TGF-β or a TGF-β receptor.

In some embodiments, the cancer over-expresses TGF-β (e.g. TGF-β1, TGF-β2 and/or TGF-β3) or a TGF-β receptor (e.g. TGF-βR1, TGF-βR2 and/or TGF-βR3). Overexpression of TGF-β or a TGF-β receptor can be determined by detection of a level of expression of TGF-β or a TGF-β receptor which is greater than the level of expression of TGF-β or a TGF-β receptor by equivalent non-cancerous cells/non-tumor tissue.

In some embodiments, a patient may be selected for treatment according to the present invention based on the detection of a cancer expressing TGF-β or a TGF-β receptor, or overexpressing TGF-β or a TGF-β receptor, e.g. in a sample obtained from the subject.

Expression may be gene expression or protein expression. Gene expression can be determined e.g. by detection of mRNA encoding TGF-β or a TGF-β receptor, for example by quantitative real-time PCR (qRT-PCR). Protein expression can be determined e.g. by detection of TGF-β or a TGF-β receptor protein, for example by antibody-based methods, for example by western blot, immunohistochemistry, immunocytochemistry, flow cytometry, or ELISA.

The cancer may have an increased level of expression or activity of a positive regulator/effector of TGF-β mediated signalling such as TGF-β (e.g. TGF-β1, TGF-β2 and/or TGF-β3) or a TGF-β receptor (e.g. TGF-βR1, TGF-βR2 and/or TGF-βR3), or may have an increased level of expression or activity of a factor upregulated by TGF-β mediated signalling. The cancer may have a reduced level of expression or activity of a negative regulator of TGF-β mediated signalling, or may have a reduced level of expression or activity of a factor downregulated by TGF-β mediated signalling. The increase/reduction may be relative to the level of expression or activity in the equivalent non-cancerous cell/tissue.

In some embodiments, the cancer may be associated with a mutation in TGF-β (e.g. TGF-β1, TGF-β2 and/or TGF-β3) or a TGF-β receptor (e.g. TGF-βR1, TGF-βR2 and/or TGF-βR3). In some embodiments, such mutation may be associated with increased level of gene or protein expression, or may be associated with an increased level of TGF-β mediated signalling relative to the level of expression/signalling observed in the absence of the mutation.

In some embodiments, the disease/condition to be treated may be one of: a disease characterised by fibrosis such as idiopathic pulmonary fibrosis (IPF), renal fibrosis, cardiac fibrosis (e.g. fibrosis associated with myocardial infarction, ischaemic, dilated and hypertrophic cardiomyopathies and congestive heart failure), scleroderma, myelodysplastic syndrome (MDS), restenosis following coronary artery bypass or angioplasty, Marfan syndrome, post-operative scarring in ocular conditions (e.g. following trabeculectomy for treatment of glaucoma, or following corneal surgery), diabetes and obesity.

In some embodiments, the disease/condition may be a T cell dysfunctional disorder. A T cell dysfunctional disorder may be a disease or condition in which normal T cell function is impaired causing downregulation of the subject's immune response to pathogenic antigens, e.g. generated by infection by exogenous agents such as microorganisms, bacteria and viruses, or generated by the host in some disease states such as in some forms of cancer (e.g. in the form of tumor associated antigens). The impaired function of T cell dysfunctional disorder may be one or more of: cytotoxicity, e.g. to a cell comprising/expressing antigen for which the T cell is specific; proliferation, IFNγ expression, CD107a expression, IL-2 expression, TNFα expression, perforin expression, granzyme expression, granulysin expression, and/or FAS ligand (FASL) expression, e.g. in response to antigen for which the T cell is specific or a cell comprising/expressing antigen for which the T cell is specific.

In some embodiments, the treatment may be aimed at increasing/enhancing one or more of the following properties of a T cell: cytotoxicity, e.g. to a cell comprising/expressing antigen for which the T cell is specific; proliferation, IFNγ expression, CD107a expression, IL-2 expression, TNFα expression, perforin expression, granzyme expression, granulysin expression, and/or FAS ligand (FASL) expression, e.g. in response to antigen for which the T cell is specific or a cell comprising/expressing antigen for which the T cell is specific. In some embodiments, the treatment may be aimed at providing or generating a T cell or population of T cells having an increased/enhanced level of one of more of the following properties: cytotoxicity, e.g. to a cell comprising/expressing antigen for which the T cell is specific; proliferation, IFNγ expression, CD107a expression, IL-2 expression, TNFα expression, perforin expression, granzyme expression, granulysin expression, and/or FASL expression, e.g. in response to antigen for which the T cell is specific or a cell comprising/expressing antigen for which the T cell is specific.

The T cell dysfunctional disorder may be manifest as an infection, or inability to mount an effective immune response against an infection. The infection may be chronic, persistent, latent or slow, and may be the result of bacterial, viral, fungal or parasitic infection. As such, treatment may be provided to patients having a bacterial, viral or fungal infection. Examples of bacterial infections include infection with Helicobacter pylori. Examples of viral infections include infection with HPV, EBV, HIV, HBV, and HCV. The T cell dysfunctional disorder may be associated with a cancer.

The treatment may be aimed at prevention of the T cell dysfunctional disorder, e.g. prevention of infection, prevention of the development or progression of a cancer. As such, the TGF-β decoy receptors, CARs, nucleic acids, vectors, cells and compositions of the present invention may be used for prophylaxis against development/progression of a disease or disease state. This may take place before the onset of symptoms of the disease or disease state, and/or may be given to subjects considered to be at greater risk development/progression of a disease or disease state.

Methods of medical treatment may also involve in vivo, ex vivo, and adoptive immunotherapies, including those using autologous and/or heterologous cells or immortalized cell lines.

### Administration

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the condition to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

TGF-β decoy receptors, CARs, nucleic acids, vectors and cells according to the present invention may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion. Suitable formulations may comprise the TGF-β decoy receptor, CAR, nucleic acid, vector, or cell in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In accordance with the present invention methods are also provided for the production of pharmaceutically useful compositions, such methods of production may comprise one or more steps selected from: isolating a TGF-β decoy receptor, CAR, nucleic acid, vector, or cell as described herein; and/or mixing a TGF-β decoy receptor, CAR, nucleic acid, vector, or cell as described herein with a pharmaceutically acceptable carrier, adjuvant, excipient or diluent.

For example, a further aspect of the present invention relates to a method of formulating or producing a medicament or pharmaceutical composition for use in a method of medical treatment, the method comprising formulating a pharmaceutical composition or medicament by mixing TGF-β decoy receptor, CAR, nucleic acid, vector, or cell as described herein with a pharmaceutically acceptable carrier, adjuvant, excipient or diluent.

Administration of a TGF-β decoy receptor, CAR, nucleic acid, vector, cell or composition according to the invention is preferably in a “therapeutically effective” or “prophylactically effective” amount, this being sufficient to show benefit to the subject. The actual amount administered, and rate and time-course of administration, will depend on the nature and severity of the disease or disorder. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Administration may be alone or in combination with other treatments, either simultaneously or sequentially dependent upon the condition to be treated. The TGF-β decoy receptor, CAR, nucleic acid, vector, cell or composition according to the present invention and a therapeutic agent may be administered simultaneously or sequentially.

In some embodiments, treatment with a TGF-β decoy receptor, CAR, nucleic acid, vector, cell or composition of the present invention may be accompanied by other therapeutic or prophylactic intervention, e.g. chemotherapy, immunotherapy, radiotherapy, surgery, vaccination and/or hormone therapy.

Simultaneous administration refers to administration of the TGF-β decoy receptor, CAR, nucleic acid, vector, cell or composition and therapeutic agent together, for example as a pharmaceutical composition containing both agents (combined preparation), or immediately after each other and optionally via the same route of administration, e.g. to the same artery, vein or other blood vessel. Sequential administration refers to administration of one of the TGF-β decoy receptor, CAR, nucleic acid, vector, cell or composition or therapeutic agent followed after a given time interval by separate administration of the other agent. It is not required that the two agents are administered by the same route, although this is the case in some embodiments. The time interval may be any time interval.

Chemotherapy and radiotherapy respectively refer to treatment of a cancer with a drug or with ionising radiation (e.g. radiotherapy using X-rays or γ-rays). The drug may be a chemical entity, e.g. small molecule pharmaceutical, antibiotic, DNA intercalator, protein inhibitor (e.g. kinase inhibitor), or a biological agent, e.g. antibody, antibody fragment, nucleic acid or peptide aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the cancer.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs and biologics may be selected from: alkylating agents such as cisplatin, carboplatin, mechlorethamine, cyclophosphamide, chlorambucil, ifosfamide; purine or pyrimidine anti-metabolites such as azathiopurine or mercaptopurine; alkaloids and terpenoids, such as vinca alkaloids (e.g. vincristine, vinblastine, vinorelbine, vindesine), podophyllotoxin, etoposide, teniposide, taxanes such as paclitaxel (Taxol™), docetaxel; topoisomerase inhibitors such as the type I topoisomerase inhibitors camptothecins irinotecan and topotecan, or the type II topoisomerase inhibitors amsacrine, etoposide, etoposide phosphate, teniposide; antitumor antibiotics (e.g. anthracyline antibiotics) such as dactinomycin, doxorubicin (Adriamycin™), epirubicin, bleomycin, rapamycin; antibody based agents, such as anti-PD-1 antibodies, anti-PD-L1 antibodies, anti-TIM-3 antibodies, anti-CTLA-4, anti-4-1 BB, anti-GITR, anti-CD27, anti-BLTA, anti-OX43, anti-VEGF, anti-TNFα, anti-IL-2, antiGpIIb/IIIa, anti-CD-52, anti-CD20, anti-RSV, anti-H ER2/neu(erbB2), anti-TNF receptor, anti-EGFR antibodies, monoclonal antibodies or antibody fragments, examples include: cetuximab, panitumumab, infliximab, basiliximab, bevacizumab (Avastin®), abciximab, daclizumab, gemtuzumab, alemtuzumab, rituximab (Mabthera®), palivizumab, trastuzumab, etanercept, adalimumab, nimotuzumab; EGFR inihibitors such as erlotinib, cetuximab and gefitinib; anti-angiogenic agents such as bevacizumab (Avastin®); cancer vaccines such as Sipuleucel-T (Provenge®).

Further chemotherapeutic drugs may be selected from: 13-cis-Retinoic Acid, 2-Chlorodeoxyadenosine, 5-Azacitidine 5-Fluorouracil, 6-Mercaptopurine, 6-Thioguanine, Abraxane, Accutane®, Actinomycin-D Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Aranesp®, Aredia®, Arimidex®, Aromasin®, Arranon®, Arsenic Trioxide, Asparaginase, ATRA Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, Calcium Leucovorin, Campath®, Camptosar®, Camptothecin-11, Capecitabine, Carac™, Carboplatin, Carmustine, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine Cytosar-U®, Cytoxan®, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Eligard™, Ellence™, Eloxatin™, Elspar®, Emcyt®, Epirubicin, Epoetin Alfa, Erbitux, Erlotinib, Erwinia L-asparaginase, Estramustine, Ethyol Etopophos®, Etoposide, Etoposide Phosphate, Eulexin®, Everolimus, Evista®, Exemestane, Faslodex®, Femara®, Filgrastim, Floxuridine, Fludara®, Fludarabine, Fluoroplex®, Fluorouracil, Fluoxymesterone, Flutamide, Folinic Acid, FUDR®, Fulvestrant, Gefitinib, Gemcitabine, Gemtuzumab ozogamicin, Gleevec™, Gliadel® Wafer, Goserelin, Granulocyte-Colony Stimulating Factor, Granulocyte Macrophage Colony Stimulating Factor, Herceptin ®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Fred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant Purinethol®, Raloxifene, Revlimid®, Rheumatrex®, Rituxan®, Rituximab, Roferon-A® (Interferon Alfa-2a), Rubex®, Rubidomycin hydrochloride, Sandostatin® Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™, Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®.

Multiple doses of the TGF-β decoy receptor, CAR, nucleic acid, vector, cell or composition may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

### Adoptive Transfer

In embodiments of the present invention, a method of treatment or prophylaxis may comprise adoptive transfer of immune cells.

Adoptive cell transfer (ACT) generally refers to a process by which cells (e.g. immune cells) are obtained from a subject, typically by drawing a blood sample from which the cells are isolated. The cells are then typically treated or altered in some way, and then administered either to the same subject or to a different subject. The treatment is typically aimed at providing population of cells with certain desired characteristics to a subject, or increasing the frequency of cells with such characteristics in that subject.

In the present invention, adoptive transfer may be performed with the aim of introducing a cell or population of cells into a subject, and/or increasing the frequency of a cell or population of cells in a subject.

Adoptive transfer of T cells is described, for example, in Kalos and June 2013, Immunity 39(1): 49-60, which is hereby incorporated by reference in its entirety. Adoptive transfer of NK cells is described, for example, in Davis et al. 2015, Cancer J. 21(6): 486-491, which is hereby incorporated by reference in its entirety.

The cell may e.g. be a neutrophil, eosinophil, basophil, dendritic cell, lymphocyte, or monocyte. The lymphocyte may be e.g. a T cell, B cell, NK cell, NKT cell or innate lymphoid cell (ILC), or a precursor thereof. In some embodiments, the cell is a T cell. In some embodiments, the T cell is a CD3+ T cell. In some embodiments, the T cell is a CD3+, CD8+ T cell. In some embodiments, the T cell is a cytotoxic T cell (e.g. a cytotoxic T lymphocyte (CTL)). In some embodiments, the T cell is a virus-specific T cell. In some embodiments, the T cell is specific for EBV, HPV, HBV, HCV or HIV.

The present invention provides a method of treating or presenting a disease or condition in a subject, the method comprising modifying at least one cell obtained from a subject to express or comprise a TGF-β decoy receptor, CAR, nucleic acid or vector according to the present invention, optionally expanding the modified at least one cell, and administering the modified at least one cell to a subject.

In some embodiments, the method comprises:
(a) isolating at least one cell from a subject;
(b) modifying the at least one cell to express or comprise a TGF-β decoy receptor, CAR, nucleic acid or vector according to the present invention,
(c) optionally expanding the modified at least one cell, and;
(d) administering the modified at least one cell to a subject.

In some embodiments, the subject from which the cell is isolated is the subject administered with the modified cell (i.e., adoptive transfer is of autologous cells). In some embodiments, the subject from which the cell is isolated is a different subject to the subject to which the modified cell is administered (i.e., adoptive transfer is of allogenic cells).

The at least one cell modified according to the present invention can be modified according to methods well known to the skilled person. The modification may comprise nucleic acid transfer for permanent or transient expression of the transferred nucleic acid.

In some embodiments, the cell may additionally be modified to comprise or express a chimeric antigen receptor (CAR), or nucleic acid or vector encoding a CAR.

Any suitable genetic engineering platform may be used to modify a cell according to the present invention. Suitable methods for modifying a cell include the use of genetic engineering platforms such as gammaretroviral vectors, lentiviral vectors, adenovirus vectors, DNA transfection, transposon-based gene delivery and RNA transfection, for example as described in Maus et al., Annu Rev Immunol (2014) 32:189-225, incorporated by reference hereinabove.

In some embodiments the method may comprise one or more of the following steps: taking a blood sample from a subject; isolating and/or expanding at least one cell from the blood sample; culturing the at least one cell in in vitro or ex vivo cell culture; introducing into the at least one cell a TGF-β decoy receptor, CAR, nucleic acid, or vector according to the present invention, thereby modifying the at least one cell; expanding the at least one modified cell; collecting the at least one modified cell; mixing the modified cell with an adjuvant, diluent, or carrier; administering the modified cell to a subject.

In some embodiments, the methods may additionally comprise treating the cell to induce/enhance expression of the TGF-β decoy receptor, CAR, nucleic acid, or vector. For example, the nucleic acid/vector may comprise a control element for inducible upregulation of expression of the TGF-β decoy receptor or CAR from the nucleic acid/vector in response to treatment with a particular agent. In some embodiments, treatment may be in vivo by administration of the agent to a subject having been administered with a modified cell according to the invention. In some embodiments, treatment may be ex vivo or in vitro by administration of the agent to cells in culture ex vivo or in vitro.

The skilled person is able to determine appropriate reagents and procedures for adoptive transfer of cells according to the present invention, for example by reference to Dai et al., 2016 J Nat Cancer Inst 108(7): djv439, which is incorporated by reference in its entirety.

In a related aspect, the present invention provides a method of preparing a modified cell, the method comprising introducing into a cell a TGF-β decoy receptor, CAR, nucleic acid or vector according to the present invention, thereby modifying the at least one cell. The method is preferably performed in vitro or ex vivo.

In one aspect, the present invention provides a method of treating or preventing a disease or condition in a subject, comprising:
(a) isolating at least one cell from a subject;
(b) introducing into the at least one cell the nucleic acid or vector according to the present invention, thereby modifying the at least one cell; and
(c) administering the modified at least one cell to a subject.

In some embodiments, the cell may additionally be modified to introduce a nucleic acid or vector encoding a chimeric antigen receptor (CAR).

In some embodiments, the method additionally comprises therapeutic or prophylactic intervention, e.g. for the treatment or prevention of a cancer. In some embodiments, the therapeutic or prophylactic intervention is selected from chemotherapy, immunotherapy, radiotherapy, surgery, vaccination and/or hormone therapy.

### Methods of Detection

TGF-β decoy receptors and CARs described herein may be used in methods that involve the binding of the TGF-β decoy receptor or CAR to TGF-β (e.g. TGF-β1, TGF-β2 or TGF-β3). Such methods may involve detection of the bound complex of TGF-β decoy receptor or CAR and TGF-β. As such, in one embodiment a method is provided, the method comprising contacting a sample containing, or suspected to contain, TGF-β with a TGF-β decoy receptor or CAR as described herein and detecting the formation of a complex of the TGF-β decoy receptor/CAR, and TGF-β.

Suitable method formats are well known in the art, including immunoassays such as sandwich assays, e.g. ELISA. The method may involve labelling the TGF-β decoy receptor/CAR, or TGF-β, or both, with a detectable label, e.g. fluorescent, luminescent or radio-label. TGF-β expression may be measured by immunohistochemistry (IHC), for example of a tissue sample obtained by biopsy. In some embodiments, the label may be selected from: a radio-nucleotide, positron-emitting radionuclide (e.g. for positron emission tomography (PET)), MRI contrast agent or fluorescent label.

Analysis in vitro or in vivo of processes mediated by TGF-β may involve analysis by positron emission tomography (PET), magnetic resonance imaging (MRI), or fluorescence imaging, e.g. by detection of appropriately labelled species.

Methods of this kind may provide the basis of a method of diagnosis of a disease or condition requiring detection and or quantitation of TGF-β. Such methods may be performed in vitro on a patient sample, or following processing of a patient sample. Once the sample is collected, the patient is not required to be present for the in vitro method of diagnosis to be performed and therefore the method may be one which is not practised on the human or animal body.

Such methods may involve determining the amount of TGF-β present in a patient sample. The method may further comprise comparing the determined amount against a standard or reference value as part of the process of reaching a diagnosis. Other diagnostic tests may be used in conjunction with those described here to enhance the accuracy of the diagnosis or prognosis or to confirm a result obtained by using the tests described here.

The level of TGF-β present in a patient sample may be indicative that a patient may respond to treatment with a TGF-β decoy receptor, CAR, nucleic acid, vector, cell or composition according to the present invention. The presence of a high level of TGF-β in a sample may be used to select a patient for treatment as described herein, e.g. with a TGF-β decoy receptor, CAR, nucleic acid, vector, cell or composition described herein. The TGF-β decoy receptors and CARs of the present invention may therefore be used to select a patient for treatment as described herein.

Detection in a sample of TGF-β may be used for the purpose of diagnosis of a disease or condition, predisposition to a disease or condition, or for providing a prognosis (prognosticating) of disease or condition. The diagnosis or prognosis may relate to an existing (previously diagnosed) disease/condition.

A sample may be taken from any tissue or bodily fluid. The sample may comprise or may be derived from: a quantity of blood; a quantity of serum derived from the individual's blood which may comprise the fluid portion of the blood obtained after removal of the fibrin clot and blood cells; a tissue sample or biopsy; pleural fluid; cerebrospinal fluid (CSF); or cells isolated from said individual. In some embodiments, the sample may be obtained or derived from a tissue or tissues which are affected by the disease/condition (e.g. tissue or tissues in which symptoms of the disease manifest, or which are involved in the pathogenesis of the disease/condition).

Methods according to the present invention may preferably be performed in vitro. The term “in vitro” is intended to encompass experiments with cells in culture whereas the term “in vivo” is intended to encompass experiments with and/or treatment of intact multi-cellular organisms.

### Kits

In some aspects of the present invention a kit of parts is provided. In some embodiments the kit may have at least one container having a predetermined quantity of a TGF-β decoy receptor, CAR, nucleic acid, vector, cell, or composition according to the present invention.

The kit may provide the TGF-β decoy receptor, CAR, nucleic acid, vector, cell or composition together with instructions for administration to a patient in order to treat a specified disease/condition. The TGF-β decoy receptor, CAR, nucleic acid, vector, cell or composition may be formulated so as to be suitable for injection or infusion to a tumor or to the blood.

In some embodiments the kit may comprise materials for producing a cell according to the present invention. For example, the kit may comprise materials for modifying a cell to express or comprise a TGF-β decoy receptor, CAR, nucleic acid or vector according to the present invention, or materials for introducing into a cell the nucleic acid or vector according to the present invention.

In some embodiments the kit may further comprise at least one container having a predetermined quantity of another therapeutic agent (e.g. anti-infective agent or chemotherapy agent). In such embodiments, the kit may also comprise a second medicament or pharmaceutical composition such that the two medicaments or pharmaceutical compositions may be administered simultaneously or separately such that they provide a combined treatment for the specific disease or condition. The therapeutic agent may also be formulated so as to be suitable for injection or infusion to a tumor or to the blood.

### Protein Expression

Molecular biology techniques suitable for producing the proteins (e.g. the TGF-β decoy receptors and CARs) according to the invention in cells are well known in the art, such as those set out in Sambrook et al., Molecular Cloning: A Laboratory Manual, New York: Cold Spring Harbor Press, 1989. Polypeptides may be expressed from a nucleic acid sequence. The nucleic acid sequence may be contained in a vector present in a cell, or may be incorporated into the genome of the cell.

Any cell suitable for the expression of polypeptides may be used for producing proteins according to the invention. The cell may be a prokaryote or eukaryote. Suitable prokaryotic cells include E. coli. Examples of eukaryotic cells include a yeast cell, a plant cell, insect cell or a mammalian cell (e.g. Chinese Hamster Ovary (CHO) cells). In some cases the cell is not a prokaryotic cell because some prokaryotic cells do not allow for the same post-translational modifications as eukaryotes. In addition, very high expression levels are possible in eukaryotes and proteins can be easier to purify from eukaryotes using appropriate tags. Specific plasmids may also be utilised which enhance secretion of the protein into the media.

Methods of producing a polypeptide of interest may involve culture or fermentation of a cell modified to express the polypeptide. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide. Culture, fermentation and separation techniques are well known to those of skill in the art.

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culture of cells that express the polypeptide of interest, that polypeptide is preferably isolated. Any suitable method for separating polypeptides from cell culture known in the art may be used. In order to isolate a polypeptide of interest from a culture, it may be necessary to first separate the cultured cells from media containing the polypeptide of interest. If the polypeptide of interest is secreted from the cells, the cells may be separated from the culture media that contains the secreted polypeptide by centrifugation. If the polypeptide of interest collects within the cell, it will be necessary to disrupt the cells prior to centrifugation, for example using sonification, rapid freeze-thaw or osmotic lysis. Centrifugation will produce a pellet containing the cultured cells, or cell debris of the cultured cells, and a supernatant containing culture medium and the polypeptide of interest.

It may then be desirable to isolate the polypeptide of interest from the supernatant or culture medium, which may contain other protein and non-protein components. A common approach to separating polypeptide components from a supernatant or culture medium is by precipitation. Polypeptides/proteins of different solubility are precipitated at different concentrations of precipitating agent such as ammonium sulfate. For example, at low concentrations of precipitating agent, water soluble proteins are extracted. Thus, by adding increasing concentrations of precipitating agent, proteins of different solubility may be distinguished. Dialysis may be subsequently used to remove ammonium sulfate from the separated proteins.

Other methods for distinguishing different polypeptides/proteins are known in the art, for example ion exchange chromatography and size chromatography. These may be used as an alternative to precipitation, or may be performed subsequently to precipitation.

Once the polypeptide of interest has been isolated from culture it may be necessary to concentrate the protein. A number of methods for concentrating a protein of interest are known in the art, such as ultrafiltration or lyophilisation.

### Sequence Identity

Pairwise and multiple sequence alignment for the purposes of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

### BRIEF DESCRIPTION OF THE FIGURES

Embodiments and experiments illustrating the principles of the invention will now be discussed with reference to the accompanying figures, in which:

FIG. 1. Amino acid sequences encoded by the TGF-β decoy receptor constructs of the present invention.

FIG. 2. Schematic representation of the mature TGF-β decoy receptors of the present invention.

FIGS. 3A to 3H. Scatterplots showing expression of TGF-β decoy receptors on the surface of transfected HeLa cells, as detected by flow cytometry using anti-human TGF-β RII antibody clone REA903. 3A to 3E show the percentage of cells expressing TGF-βR2 ectodomain at the cell surface following transfection with vector encoding DN-TGF-βR2 (3A), TGF-βRII-PLAC ALKPHOS GPI anchor (3B), TGF-βRII-CD48 GPI anchor (3C), TGF-βRII-CD55 GPI anchor (3D) or TGF-βRII-CD90 GPI anchor (3E). 3F and 3G show the percentage of cells expressing TGF-βR2 ectodomain at the cell surface of wildtype, non-transfected HeLa cells stained with anti-human TGF-β RII antibody (3F) or unstained (3G). 3H shows the percentage of cells expressing GFP following transfection with vector encoding GFP.

FIG. 4. Scatterplots showing expression of TGF-β decoy receptors on the surface of transduced activated T cells, as detected by flow cytometry using anti-human TGF-β RII antibody clone REA903. 4A to 4E show the percentage of cells expressing TGF-βR2 ectodomain at the cell surface following transduction with lentivirus encoding DN-TGF-βR2 (4A), TGF-βRII-PLAC ALKPHOS GPI anchor (4B), TGF-βRII-CD48 GPI anchor (4C), TGF-βRII-CD55 GPI anchor (4D) or TGF-βRII-CD90 GPI anchor (4E). 4F shows the percentage of cells expressing TGF-βR2 ectodomain at the cell surface of wildtype, non-transduced activated T cells stained with anti-human TGF-β RII antibody. 4G and 4H shows the percentage of cells expressing GFP following transduction with lentivirus encoding GFP, of activated T cells stained with anti-human TGF-β RII antibody (4G) or unstained (4H).

FIGS. 5A to 5C. Graphs showing the percentages of leukocytes expressing different proteins in non-transduced activated T cells (wt), or activated T cells transduced with lentivirus encoding GFP (GFP) or TGF-βRII-CD90 GPI anchor (CD90), as determined by flow cytometry. 5A shows the total number of leukocytes, 5B shows the percentage of GFP+cells, and 5C shows the percentage of cells expressing TGF-βR2 ectodomain at the cell surface.

FIGS. 6A and 6B. Graphs showing the percentages of CD3+ T cells expressing different proteins as a proportion of the total leukocyte population in non-transduced activated T cells (wt), or activated T cells transduced with lentivirus encoding GFP (GFP), DN-TGF-βR2 (DN), TGF-βRII-CD48 GPI anchor (CD48) or TGF-βRII-CD90 GPI anchor (CD90), as determined by flow cytometry. 5A shows the percentages of CD3+ T cells and 6B shows the percentage of GFP+ CD3+ T cells.

FIGS. 7A to 7D. Graphs showing the percentages of CD4 and CD8 T cell subsets expressing different proteins as a proportion of the CD3+ cell population in non-transduced activated T cells (wt), or activated T cells transduced with lentivirus encoding GFP (GFP) or TGF-βRII-CD90 GPI anchor (CD90), as determined by flow cytometry. 7A shows the percentage of CD4+, GFP+ cells, 7B shows the percentage of CD8+, GFP+ cells, 7C shows the percentage of CD4+ cells expressing TGF-βR2 ectodomain at the cell surface, and 7D shows the percentage of CD8+ cells expressing TGF-βR2 ectodomain at the cell surface.

FIGS. 8A to 8E. Graphs showing the percentages of HeLa cells expressing different proteins as a proportion of the total cell population in non-transduced HeLa cells (wt), or HeLa cells transduced with lentivirus encoding GFP (GFP) or TGF-βRII-CD90 GPI anchor (CD90), following stimulation for 30 min with TGF-β at the indicated concentrations, as determined by flow cytometry. 8A shows the percentage of cells staining positive for phosphorylated SMAD2/3, 8B shows the percentage of cells staining positive for phosphorylated SMAD1/5/9, 8C shows the percentage of cells staining positive for NFκB p65, 8D shows the percentage of cells staining positive for phosphorylated MAPKK, and 8E shows the percentage of cells staining positive for CREB.

FIGS. 9A to 9C. Graphs showing the percentages of CD8+ cells expressing different proteins as a proportion of the CD3+ cell population in non-transduced activated T cells (wt), or activated T cells transduced with lentivirus encoding GFP (GFP), DN-TGF-βR2 (DN), TGF-βRII-CD48 GPI anchor (CD48) or TGF-βRII-CD90 GPI anchor (CD90), following stimulation for 30 min with TGF-β at the indicated concentrations, as determined by flow cytometry. 9A shows the percentage of CD3+ cells staining positive for CD8 and phosphorylated SMAD2/3, 9B shows the percentage of CD3+ cells staining positive for CD8 and NFκB p65, and 9C shows the percentage of CD3+ cells staining positive for CD8 and CREB.

FIGS. 10A to 10C. Graphs showing the percentages of CD4+ cells expressing different proteins as a proportion of the CD3+ cell population in non-transduced activated T cells (wt), or activated T cells transduced with lentivirus encoding GFP (GFP), DN-TGF-βR2 (DN), TGF-βRII-CD48 GPI anchor (CD48) or TGF-βRII-CD90 GPI anchor (CD90), following stimulation for 30 min with TGF-β at the indicated concentrations, as determined by flow cytometry. 10A shows the percentage of CD3+ cells staining positive for CD4 and phosphorylated SMAD2/3, 10B shows the percentage of CD3+ cells staining positive for CD4 and NFκB p65, and 10C shows the percentage of CD3+ cells staining positive for CD4 and CREB.

FIG. 11. Images showing the results of western blot analysis of levels of phosphorylated SMAD2/3 in non-transduced activated T cells (wt), or activated T cells transduced with lentivirus encoding TGF-βRII-CD90 GPI anchor (CD90) following stimulation for 30 min with TGF-β at the indicated concentrations. Loading control is shown.

FIG. 12. Images showing the results of western blot analysis of levels of phosphorylated SMAD2/3 in non-transduced HeLa cells (wt), or HeLa cells transduced with lentivirus encoding GFP (GFP) or TGF-βRII-CD90 GPI anchor (CD90) following stimulation for 30 min with TGF-β at the indicated concentrations. Loading control is shown.

FIGS. 13A and 13B. Graphs showing the results of analysis by fluorescence microscopy of the level of (13A) phosphorylated p38 and (13B) phosphorylated SMAD2 localised to the nucleus of non-transduced HeLa cells (WT), and HeLa cells transduced with lentivirus encoding TGF-βRII-CD48 GPI anchor (48), TGF-βRII-CD90 GPI anchor (90), or DN-TGF-βR2 (DN), following stimulation with TGF-β.

### EXAMPLES

In the following Examples, the inventors describe the design of TGF-β binding decoy receptor molecules, and their functional characterisation.

### Example 1

### TGF-β Decoy Receptor Design

A lentiviral vector is used to prepare TGF-β decoy receptor constructs encoding the TGF-β binding domain of the type II receptor for TGF-β and different cell membrane anchor regions.

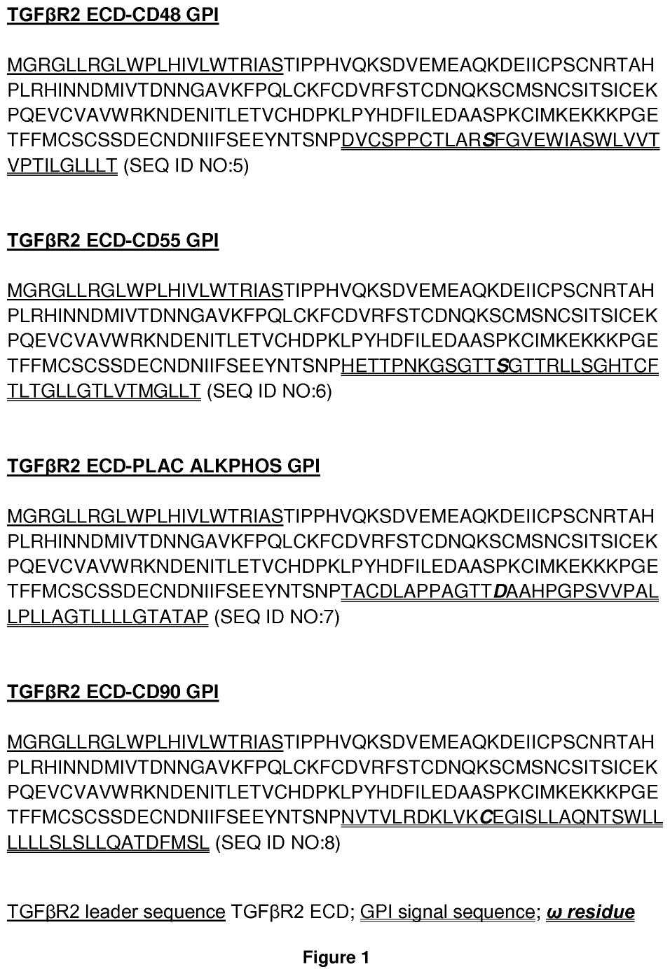

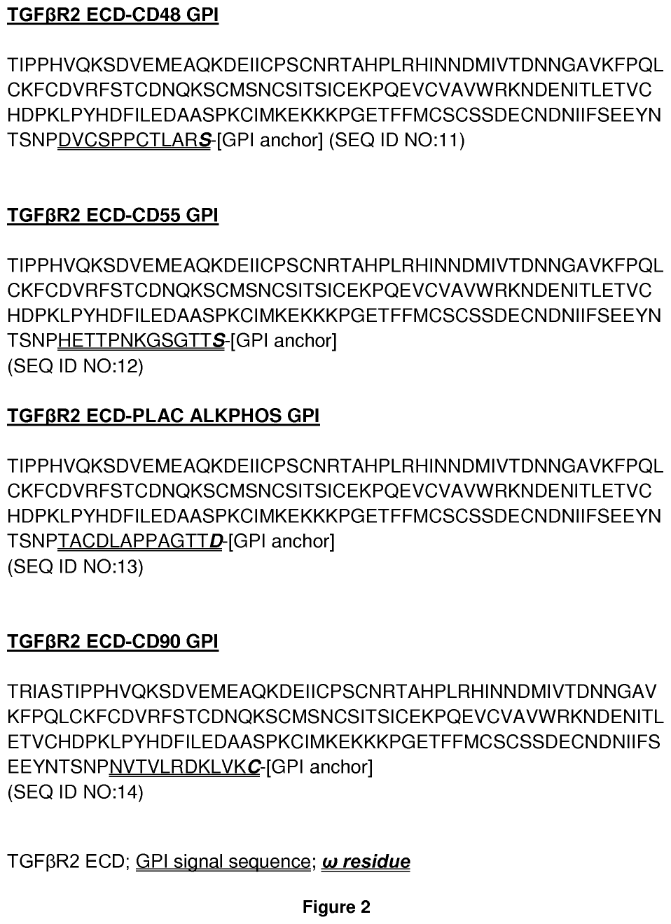

The cDNA for the extracellular domain of type II receptor for TGF-β is cloned in-frame with cDNA encoding the glycosylphosphatidylinositol (GPI) anchor sequences for CD48, CD44, CD55, CD90 or placental-type alkaline phosphatase (amino acid sequences encoded by the constructs are shown in FIG. 1).

### Example 2

### Generation of TGF-β Decoy Receptor Expressing Human T Lymphocytes

For lentiviral transduction, 5×10⁶ HEK 293T cells are plated on 10 cm² dish pre-coated with 0.002% poly-L-lysine (Sigma, St. Louis Mo.). The lentiviral vector is co-transfected with plasmids encoding packaging and envelope genes, and several days after co-transfection virus-containing supernatant is collected and passed through a 0.45 μm filter. The supernatant is then concentrated by ultracentrifugation at 25,000 rpm, titered, and then stored at −80° C. until use.

Primary human T lymphocytes isolated from healthy donors are acquired. T cells are cultured in complete medium (RPMI 1640 supplemented with 10% inactivated FBS, penicillin and streptomycin sulfate), and activated by stimulation with anti-CD3 and anti-CD28mAb-coated beads (Invitrogen). 12 hours after activation, the T cells are transduced with lentiviral vectors in presence of polybrene. Human T lymphocytes are expanded and maintained by addition of IL-2 every other day.

### Example 3

### Functional Characterisation of T Cells Expressing the TGF-β Decoy Receptors

### 3.1 Surface Expression

T cells modified to express TGF-β decoy receptors are analysed for surface expression of the decoy receptors.

Briefly, T cells are transduced with constructs encoding TGF-β decoy receptors according to the present invention, a negative control construct, or construct encoding DN-TGF-βR2, and expression at the cell surface is analysed by flow cytometry analysis using an antibody capable of specific binding to the extracellular domain of TGF-βR2.

T cells transduced with constructs encoding the TGF-β decoy receptors of the present disclosure display more surface staining with anti-TGF-βR2 antibody as compared to T cells transduced with the negative control construct, or construct encoding DN-TGF-βR2.

### 3.2 Soluble Expression

T cells modified to express TGF-β decoy receptors are analysed for soluble expression of the decoy receptors.

Briefly, T cells are transduced with constructs encoding TGF-β decoy receptors according to the present invention, a negative control construct, or construct encoding DN-TGF-βR2, and soluble expression of the receptor is measured by ELISA of the cell culture supernatant using antibody capable of specific binding to the extracellular domain of TGF-βR2.

T cells transduced with constructs encoding the TGF-β decoy receptors of the present disclosure secrete more soluble receptor as compared to T cells transduced with the negative control construct, or construct encoding DN-TGF-βR2.

### 3.3 TGF-β Mediated Signalling

T cells modified to express TGF-β decoy receptors are analysed for ability to respond to stimulation with TGF-β.

Briefly, T cells are transduced with constructs encoding TGF-β decoy receptors according to the present invention, a negative control construct, or construct encoding DN-TGF-βR2. The cells are then stimulated by treatment with TGF-β, and the level of TGF-β mediated signalling is analysed.

T cells transduced with constructs encoding the TGF-β decoy receptors of the present disclosure display less TGF-β mediated signalling as compared to T cells transduced with the negative control construct, or construct encoding DN-TGF-βR2.

### 3.4 Proliferation/Expansion

T cells modified to express TGF-β decoy receptors are analysed for ability to proliferate in vitro.

Briefly, T cells are transduced with constructs encoding TGF-β decoy receptors according to the present invention, a negative control construct, or construct encoding DN-TGF-βR2. The cells are then stimulated by treatment with TGF-β, and proliferation of the cells is analysed.

T cells transduced with constructs encoding the TGF-β decoy receptors of the present disclosure proliferate more as compared to T cells transduced with the negative control construct, or construct encoding DN-TGF-βR2.

### 3.5 Survival

T cells modified to express TGF-β decoy receptors are analysed for ability to persistence/survival.

Briefly, T cells are transduced with constructs encoding TGF-β decoy receptors according to the present invention, a negative control construct, or construct encoding DN-TGF-βR2. The cells are then stimulated by treatment with TGF-β, and cell numbers are monitored over time.

T cells transduced with constructs encoding the TGF-β decoy receptors of the present disclosure are found to have increased survival as compared to T cells transduced with the negative control construct, or construct encoding DN-TGF-βR2.

### 3.6 Cytotoxicity

T cells modified to express TGF-β decoy receptors are analysed for cytotoxicity in vitro.

Briefly, T cells are transduced with constructs encoding TGF-β decoy receptors according to the present invention, a negative control construct, or construct encoding DN-TGF-βR2. The cells are then stimulated by treatment with TGF-β, and the cytotoxicity to cells expressing antigen for which the T cells are specific is analysed. The cells are also analysed for gene or protein expression of cytotoxic/effectors factors.

T cells transduced with constructs encoding the TGF-β decoy receptors of the present disclosure display improved cytotoxicity and increased expression of cytotoxic/effectors factors as compared to T cells transduced with the negative control construct, or construct encoding DN-TGF-βR2.

### 3.7 Ability to Kill Tumor Cells In Vitro

T cells modified to express TGF-β decoy receptors are analysed for ability to kill tumour cell in vitro.

Briefly, T cells are transduced with constructs encoding TGF-β decoy receptors according to the present invention, a negative control construct, or construct encoding DN-TGF-βR2. The modified cells are then analysed for their ability to lyse tumour cells in vitro.

T cells expressing TGF-β decoy receptors of the present disclosure are found to kill tumour cells more quickly as compared with T cells transduced with the negative control construct, or construct encoding DN-TGF-βR2. This is observed even after normalising for effector cell:tumour cell ratios.

### 3.8 Treatment of Cancer In Vivo

T cells modified to express TGF-β decoy receptors are analysed for ability to treat cancer in vivo in a mouse model.

Briefly, T cells are transduced with constructs encoding TGF-β decoy receptors according to the present invention, a negative control construct, or construct encoding DN-TGF-βR2. The modified cells are then administered to mice having an established cancer model, and cancer progression and survival is monitored.

Mice treated with T cells transduced with constructs encoding the TGF-β decoy receptors of the present disclosure are found to have improved survival or reduced severity of disease progression as compared with T cells transduced with the negative control construct, or construct encoding DN-TGF-βR2.

### Example 4

### TGF-β Decoy Receptors Characterised

An overview of the TGF-β decoy receptor molecules characterised in the following Examples is provided below:

| Name | Receptor |
|---|---|
| DN TGFBR2 | TGF-βR2 ECD-TGF-βR2 transmembrane region |
| TGFBR2 ECTO CD48 GPI | TGFβR2 ECD-CD48 GPI (SEQ ID NO: 5) |
| TGFBR2 ECTO CD55 GPI | TGFβR2 ECD-CD55 GPI (SEQ ID NO: 6) |
| TGFBR2 ECTO ALKPHOS PLAC GPI | TGFβR2 ECD-PLAC ALKPHOS GPI (SEQ ID NO: 7) |
| TGFBR2 ECTO CD90 GPI | TGFβR2 ECD-CD90 GPI (SEQ ID NO: 8) |
| HA DN TGFBR2 | HA-tag-DN-TGF-βR2 |
| HA TGFBR2 ECTO CD48 GPI | HA-tag-TGFβR2 ECD-CD48 GPI |
| HA TGFBR2 ECTO CD55 GPI | HA-tag-TGFβR2 ECD-CD55 GPI |
| HA TGFBR2 ECTO ALKPHOS PLAC GPI | HA-tag-TGFβR2 ECD-PLAC ALKPHOS GPI |

The lentiviral vector pD2109-EFs: EF1a-ORF, Lenti-ElecD (ATUM, Newark, Calif., USA) was used to prepare constructs encoding the TGF-β decoy receptors, and a control construct encoding GFP.

### Example 5

### Preparation of Activated T Cells (ATCs)

PBMCs were purified by Ficoll gradient from blood samples obtained from different donors. CD3+ T cells were subsequently positively selected from the PBMCs using CD3 magnetic beads (Miltenyi).

The T cells were then stimulated. Briefly, 1×10⁶ cells T cells were seeded in 1 ml of CTL media (50% RPMI, 50% CLICK media, 10% FBS) containing IL-2 (40 U/ml) and CD3/CD28 Dynabeads (Gibco) for 24 to 72 hrs to obtain activate T cells (ATCs).

### Example 6

### Production of Lentivirus and Transduction of Target Cells

### 6.1 Production of Lentivirus

1.2-2.4×10⁶ 293T cells were plated in 10 cm² cell culture dishes in 10 ml of DMEM medium supplemented with 10% FBS and antibiotics.

The next day a DNA mix was prepared, comprising 3 μg of the lentiviral vector construct DNA, 1.5 μg of the envelope plasmid pMD2.G, 1.5 μg of the packaging plasmid pRSV-REV and 1.5 μg of the packaging plasmid pMDLg/pRRE. A separate mix of 18 μl of Fugene 6 (Roche) and 270 μl of FBS-free DMEM or Opti-MEM was prepared, mixed by vortexing for 10 seconds, centrifuged and incubated for 5 min at room temperature. The DNA and Fugene mixtures were then combined, mixed by vortexing for 10 sections, centrifuged and incubated for 15-45 min at room temperature. The mixture was then added to the cultures of 293T cells.

The next day, the cell culture medium was removed from the cells in culture, and 8-10 ml of fresh cell culture medium was added to the cells. The fresh cell culture medium was selected according to the cell type to be transduced with the virus. For example, RPMI-1640 medium was used where the virus was to be used to transduce T cells.

The next day, the lentivirus-containing cell culture medium was collected and stored at 4° C., and 8-10 ml fresh cell culture medium was added to the cells. On the following day, the lentivirus-containing cell culture medium was again collected and combined with the lentivirus-containing cell culture medium collected on the previous day. The mixture was then filtered using a 0.45 pm nitrocellulose membrane, and polybrene was added to a final concentration of 5-8 μg/ml. The lentivirus-containing medium was then either used for transductions immediately, or stored at −80° C.

In some cases the lentivirus-containing medium was concentrated 10-20 times using Amicon Ultra-100 tubes (Millipore). Briefly, tubes were sterilised by UV light treatment for 1 h or overnight and lentivirus containing medium was added to the tubes and centrifuged at 2,500-3,500rpm for 10-30 min.

### 6.2 Transduction of Target Cells

Briefly, target cells in culture were harvested and 0.25-1×10⁶ cells were resuspended in 0.5-1 ml of the lentivirus-containing cell culture medium (see Example 6.1). The mixture was then transferred to 24 well or 12 well plates, and centrifuged at 2,500 rpm for 1.5 hours at 30-35° C. (referred to as ‘spinfection’. The cells were then cultured overnight in an incubator (5% CO₂, 37° C.). The next day the cells were harvested and resuspended in 5-10 ml of fresh cell culture medium. In some cases puromycin was added to the cells in culture 36-48 hours after transduction, to select for transductants.

Activated T cells (ACTs) were transduced as follows. 2.5×10⁵ ATCs were resuspended in 500 μl of concentrated viral supernatant (10× concentrated, MOI up to 7000) containing 8 μg/ml polybrene, seeded in a 24 well plate and centrifuged at 800 g, 32° C. for 90 min. After spinfection, the cells were incubated at 37° C., 5% CO2 for up to 24 hrs before the supernatant was replaced by CTL medium containing IL-2 and CD3/CD28 Dynabeads (see Example 5).

For Hela cell transductions, 4×10⁵ HeLa cells were resuspended in 1 ml of concentrated viral supernatant (10× concentrated, MOI up to 7000) containing 8 μg/ml polybrene, seeded in a 24 well plate and centrifuged at 800 g, 32° C. for 90 min.

### Example 7

### Analysis of Expression of TGF-β Decoy Receptors at the Cell Surface

7.1 Expression by HeLa Cells Transfected with Constructs Encoding TGF-β Decoy Receptors

The inventors investigated surface expression of the TGF-β decoy receptors on HeLa cells transfected with different constructs.

Briefly, HeLa cells were transfected with constructs encoding TGF-β decoy receptors, a construct encoding GFP as a transfection control, or a construct encoding DN-TGF-βR2 using Lipofectamine according to the manufacturer's instructions.

72 hours after transfection cells were analysed by flow cytometry for expression of the receptor at the cell surface using anti-human TGF-β RII antibody clone REA903 (#130-115, Miltenyi), which binds to the extracellular domain of TGF-βR2. The transfection control was analysed for GFP expression.

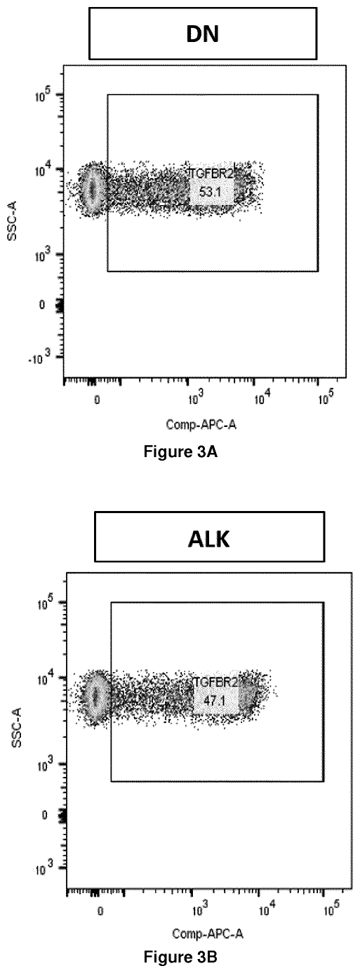

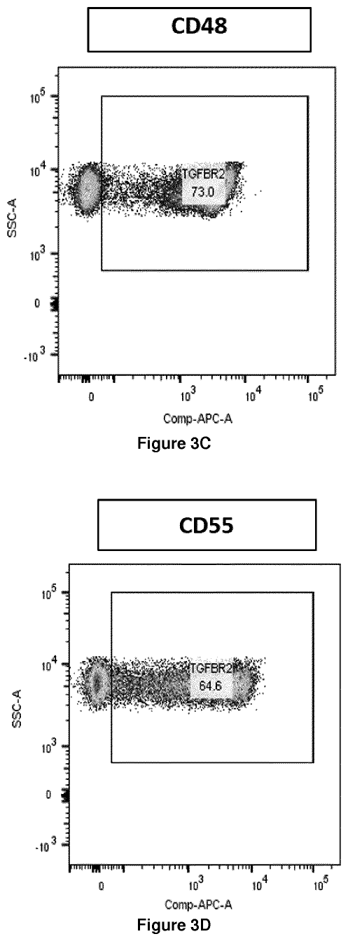

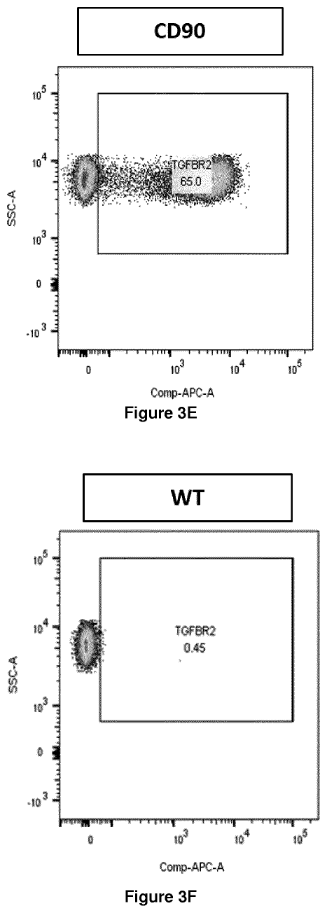

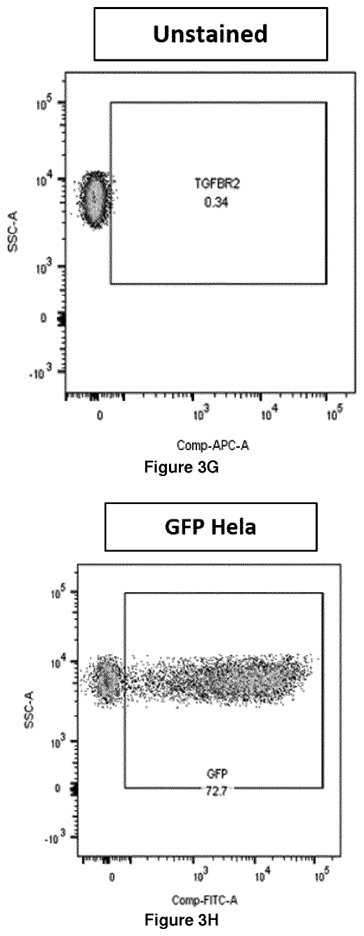

The results are shown in FIGS. 3A to 3H. High expression of the TGF-β decoy receptors was detected on the surface of HeLa cells 72 hours after transfection (˜60-70% of cells were positive for receptor expression).

7.2 Expression by Activated T Cells (ATCs) Transduced with Constructs Encoding TGF-β Decoy Receptors

The inventors next investigated surface expression of the TGF-β decoy receptors on ATCs transduced with different lentiviral constructs, or expression on non-transduced cells.

72 hours after transduction (see Example 6.2) cells were analysed by flow cytometry for expression of the receptor at the cell surface using anti-human TGF-β RII antibody clone REA903. Control conditions were analysed for GFP expression.

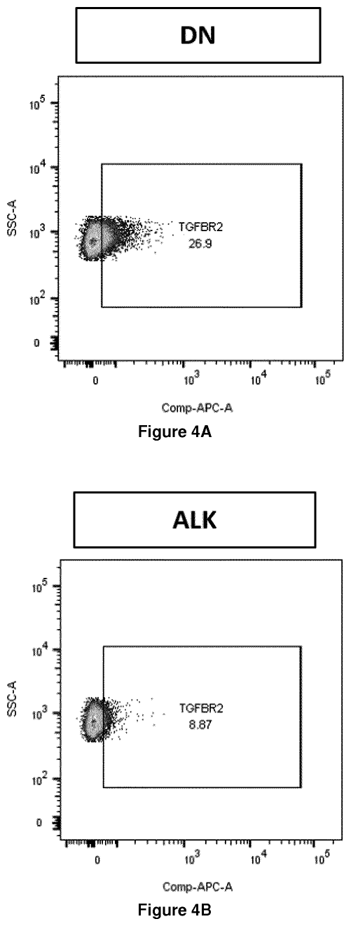

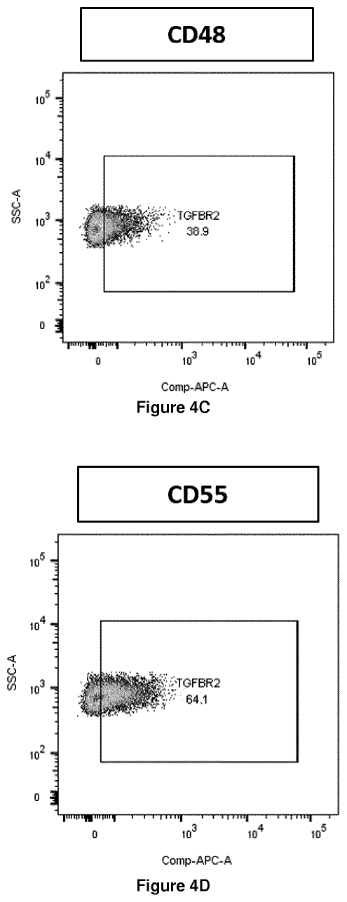

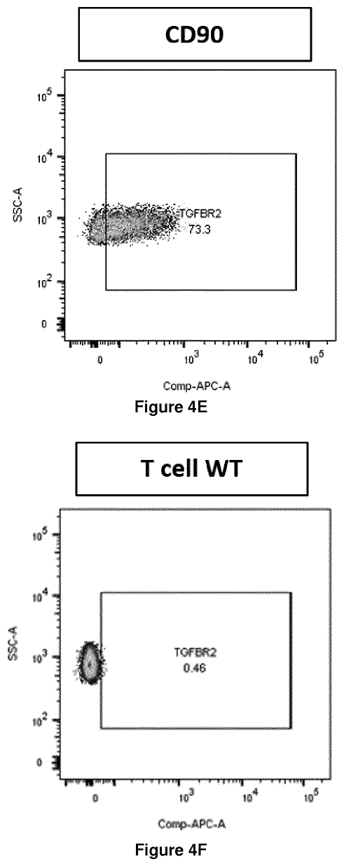

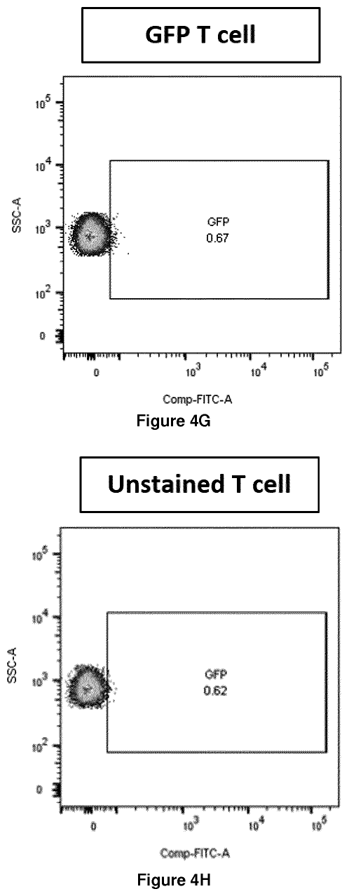

The results are shown in FIGS. 4A to 4H. ATCs transduced with the TGF-β decoy receptors encoding CD48, CD55 and CD90 GPI anchor sequences were found to display the highest level of TGF-β decoy receptor expression at the cell surface. The expression of these decoy receptors was higher than the level of expression detected for cells transduced with the construct encoding DN-TGF-βR2 (FIG. 4A).

The inventors next investigated expression of TGF-β decoy receptors having CD48 or CD90 GPI anchor sequences by transduced ATCs derived from three different donors, at 72 hours after transduction as described in Example 6.2.

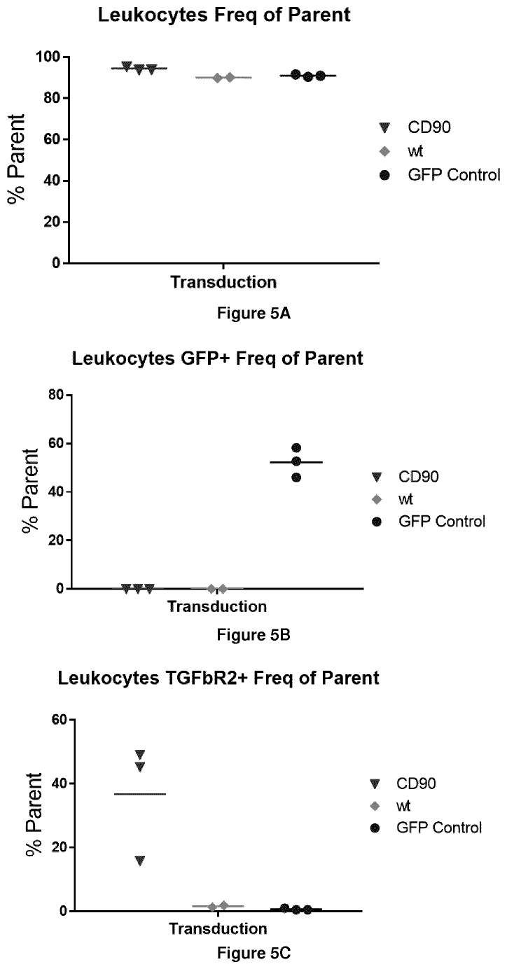

FIG. 5A shows the percentage of leukocytes in the transduced cell culture. FIG. 5B shows the percentage of GFP+ cells in transduced cell culture. FIG. 5C shows the percentage of TGF-β RII+ cells in the transduced cell culture.

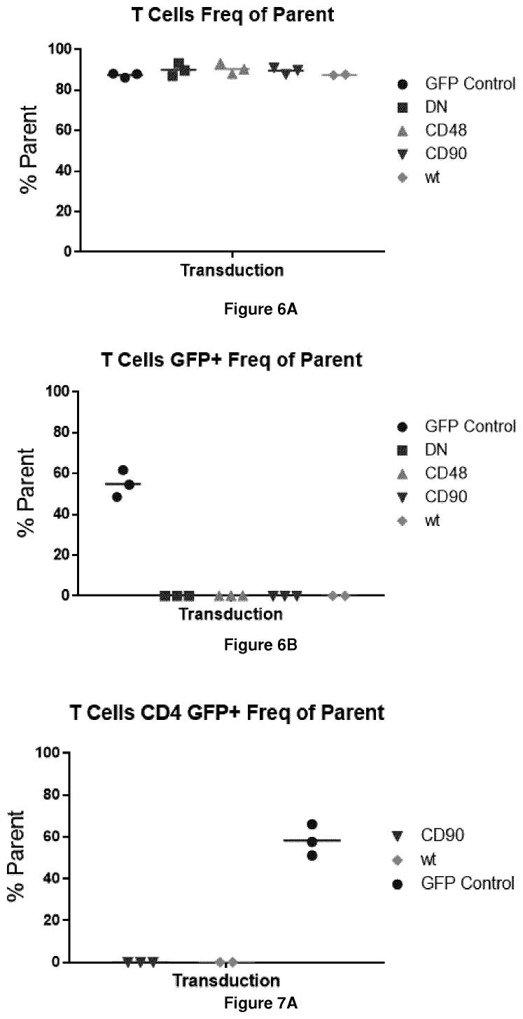

FIG. 6A shows the percentage of CD3+ cells as a proportion of the total number of leukocytes, and FIG. 6B shows the percentage of GFP+CD3+ cells as a proportion of the total number of leukocytes.

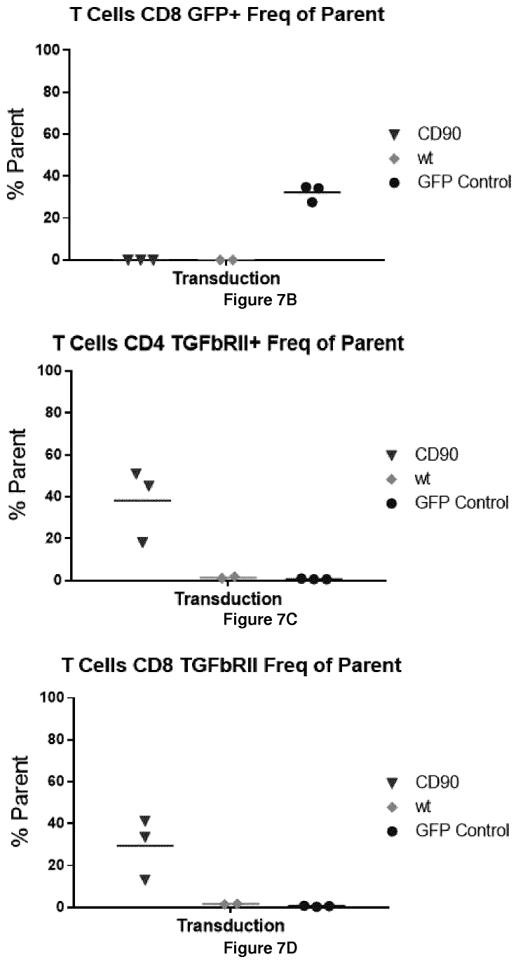

FIG. 7A shows the percentage of CD4+, GFP+ cells as a proportion of the CD3+ cell population. FIG. 7B shows the percentage of CD8+, GFP+ cells as a proportion of the CD3+ cell population. FIG. 7C shows the percentage of CD4+, TGF-β RII+ cells as a proportion of the CD3+ cell population. FIG. 7D shows the percentage of CD8+, TGF-β RII+ cells as a proportion of the CD3+ cell population.

A high proportion of cells expressing TGF-β RII was detected amongst cells transduced with lentivirus encoding TGF-βRII-CD90 GPI anchor (see FIGS. 5C, 7C and 7D).

### Example 8

### Analysis of Inhibition of TGF-β Mediated Signalling in Cells Expressing TGF-β Decoy Receptors

The inventors next investigated activation of downstream signalling in cells expressing TGF-β decoy receptors in response to stimulation with TGF-β.

Cells were transduced with lentivirus encoding TGF-βRII-CD90 GPI anchor, or construct encoding GFP (see Example 6.2).

72 hours after transduction cells puromycin was added to the cell culture at a final concentration of 2 μg/ml to select for transduced cells.

After 7 days of culture in the presence of puromycin, cells were resuspended in fresh cell culture medium and cultured for 24 hours prior to stimulation by treatment with different concentrations of TGF-β. Cells were stimulated for 30 min at 37° C., 5% CO₂. Cells were then harvested and analysed for phosphorylation of intracellular signalling proteins by flow cytometry (see Example 8.1) and by western blot (see Example 8.2). Preparation of samples for flow cytometry or western blot analysis was performed on ice.

### 8.1 Phosflow Analysis

The following antibodies were used in the flow cytometry analysis:

| | | Company- |
|---|---|---|
| Fluorochrome-Antigen | Clone | Catalog |
| PECF594 - CD56 | NCAM16.2 (also known | BD 564849 |
| | as NCAM 16) | |
| PC5-CD16 | 3G8 | BL 302010 |
| BV421- CD3 | SP34-2 | BD 562877 |
| BV786- CD4 | SK3 | BD 563877 |
| BV650- CD8 | RPA-T8 | BD 563821 |
| BV711- CD14 | M5E2 | BL 301838 |
| FITC-p38 | 36/p38 (pT180/pY182) | BD 612594 |
| PE-pERK1/2 | 20A | BD 612566 |
| PECy7-p65 NFkB | K10-895.12.50 | BD 560335 |
| AF647-pSAPK | (Thr183/Tyr185) (G9) | Cell Signaling |
| | | #9257 |
| APCy7-L/D | | ThermoFisher |
| | | L10119 |

100 μl of stimulated cells (200,000 cells) were transferred to wells of a 96 well plate. Cells were then centrifuged for 30 seconds, 3000 rpm at 4° C., the cell pellet was resuspended in 30 μL of 1:200 live/dead stain and cells were incubated for 30 seconds at room temperature. Cells were then washed by addition of 100 μL staining buffer and centrifugation for 30 seconds, 3000 rpm at 4° C. The cell pellet was then resuspended in 1:1 mix of cell culture media and BD phosflow Buffer I (50 μl per well) pre-warmed to 37° C. and incubated at 37° C. for 10 min. Cells were then harvested by centrifugation for 5 min at 1500 rpm, and washed twice with 150 μL staining buffer. The cell pellet was then resuspended in 50 μL Perm Buffer III, and cells were incubated on ice for 30 min. Cells were then harvested by centrifugation for 5 min at 1500 rpm, and washed twice with 150 μL staining buffer. The cell pellet was then resuspended in 25 μL antibody cocktail and incubated at room temperature for 1 hour in the dark. Cells were then harvested by centrifugation for 5 min at 1500 rpm, and washed twice with 150 μL staining buffer. Finally, the cell pellet was resuspended in 150 μL staining buffer, and cells were analysed by flow cytometry.

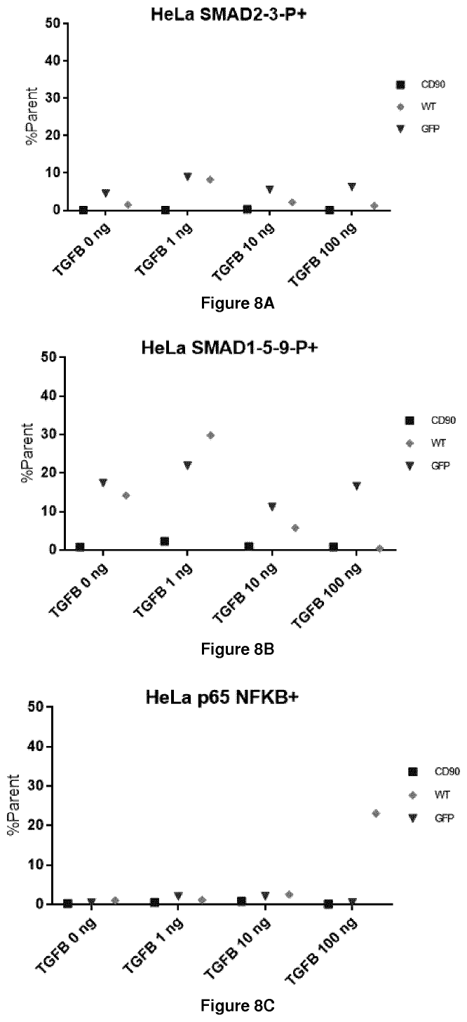

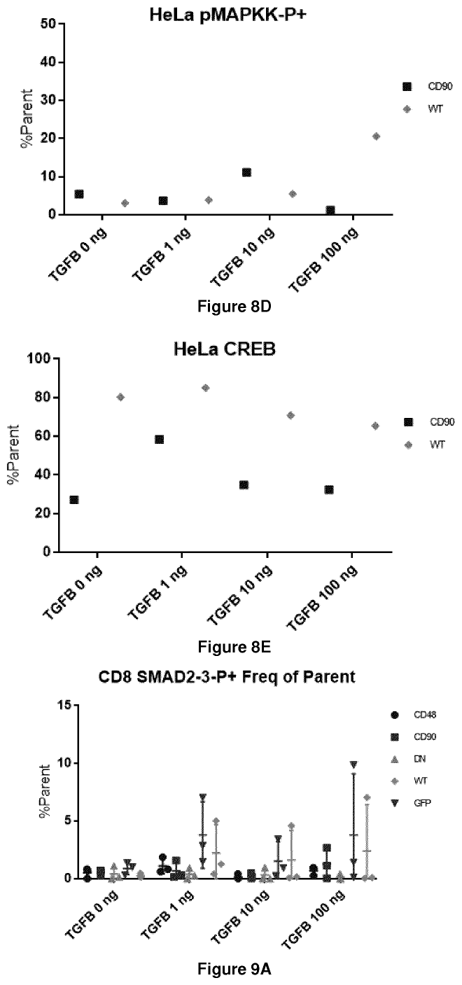

FIGS. 8A to 8E show the results of analysis of signalling in non-transduced HeLa cells (wt), HeLa cells transduced with lentivirus encoding TGF-βRII-CD90 GPI anchor (CD90) or HeLa cells transduced with lentivirus encoding GFP (GFP), following stimulation with different concentrations of TGF-β. Cells transduced with TGF-βRII-CD90 GPI anchor displayed less activation of TGF-β mediated signalling in response to stimulation with TGF-β as evidenced by reduced percentages of cells staining positive for phosphorylated signalling proteins (SMADs), and reduced percentages of cells staining positive for CREB transcription factor.

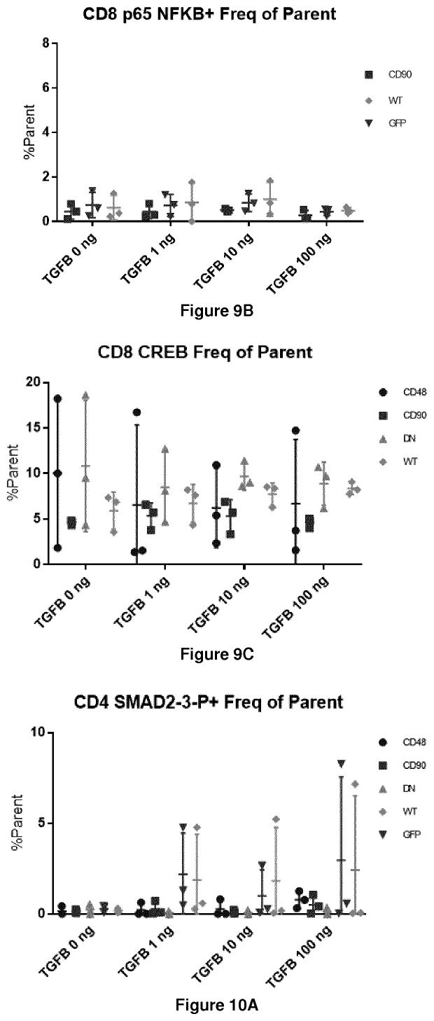

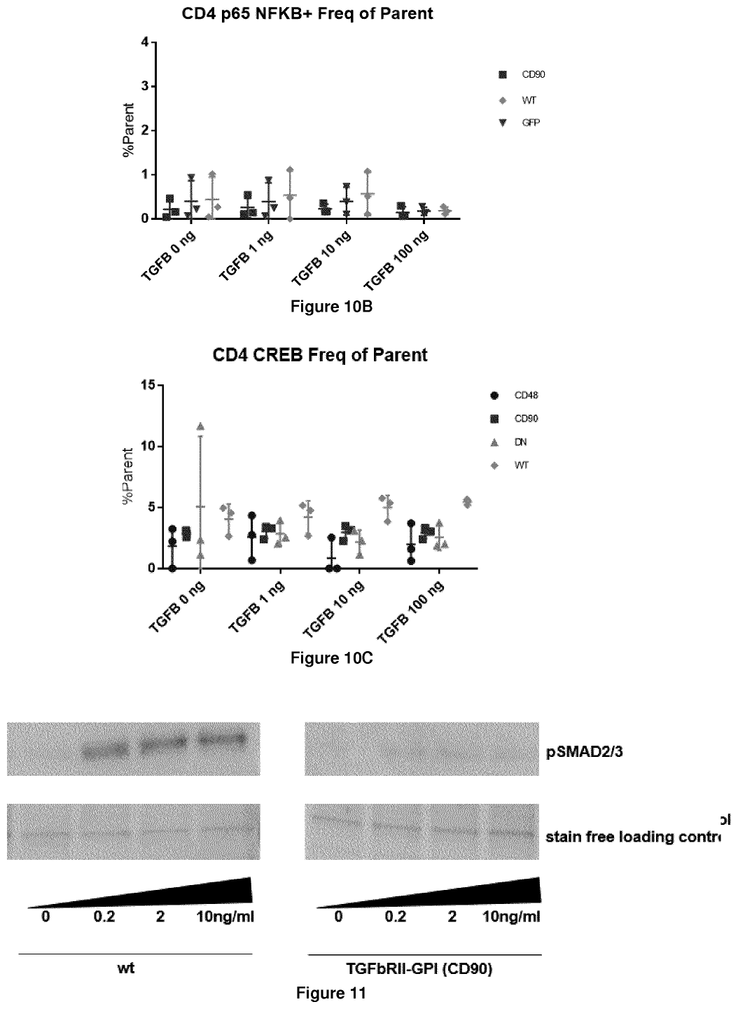

FIGS. 9 and 10 show the results of analysis of signalling in non-transduced ATCs (wt), ATCs transduced with lentivirus encoding TGF-βRII-CD90 GPI anchor (CD90), ATCs transduced with lentivirus encoding TGF-6RII-CD48 GPI anchor (CD48), ATCs transduced with lentivirus encoding DN-TGF-βR2 (DN) or ATCs transduced with lentivirus encoding GFP (GFP), following stimulation with different concentrations of TGF-β. FIGS. 9A to 9C show the percentage of CD8+ cells staining positive for the different markers within the CD3+ cell population, and FIGS. 10A to 10C show the percentage of CD4+ cells staining positive for the different markers within the CD3+ cell population.

Cells transduced with TGF-β decoy receptor having GPI anchor sequences displayed similar or reduced levels of TGF-β mediated signalling in response to stimulation with TGF-β as compared to cells transduced with lentivirus encoding DN-TGF-βR2.

### 8.2 Western Blot

The stimulated cells were resuspended in ice-cold PBS, pelleted by centrifugation at 4° C. and washed twice in ice-cold PBS. The cell pellet was then resuspended in ice-cold RIPA lysis buffer containing Phosphatase/Protease inhibitor cocktail (1:100, ThermoFisher, #78440) and incubated for 30 min on ice with occasional vortexing. The protein concentration was then measured using the BCA protein assay kit (Pierce) and concentrations were normalised between samples. 4× Laemmli buffer containing 10% MeEtOH was then added to the samples, which were then boiled at 95° C. for 10 min. Samples were then cooled on ice and then subjected to SDS-PAGE.

Nitrocellulose membranes were blocked in 5% milk solution for 2 hrs, followed by a 12 hrs incubation with primary antibody (a-Smad2/3 Antibody, Cell Signaling, #5678) at a dilution of 1:1000. Membranes were then washed 3 times in TBS-T solution, followed by incubation with secondary anti-rabbit-HRP antibody at a dilution of 1:10 000. Membranes were then washed 3 times in TBS-T solution and developed using Enhanced Chemi-luminescence (ECL, Biorad). Bands were visualised using the Chemidoc MP Imaging system (Bio-Rad).

FIG. 11 shows phosphorylated SMAD2/3 detected in primary human T cells activated by anti-CD3/CD28 stimulation and transduced with lentivirus encoding TGF-βRII-CD90 GPI anchor, or non-transduced cells, following stimulation with TGF-β.

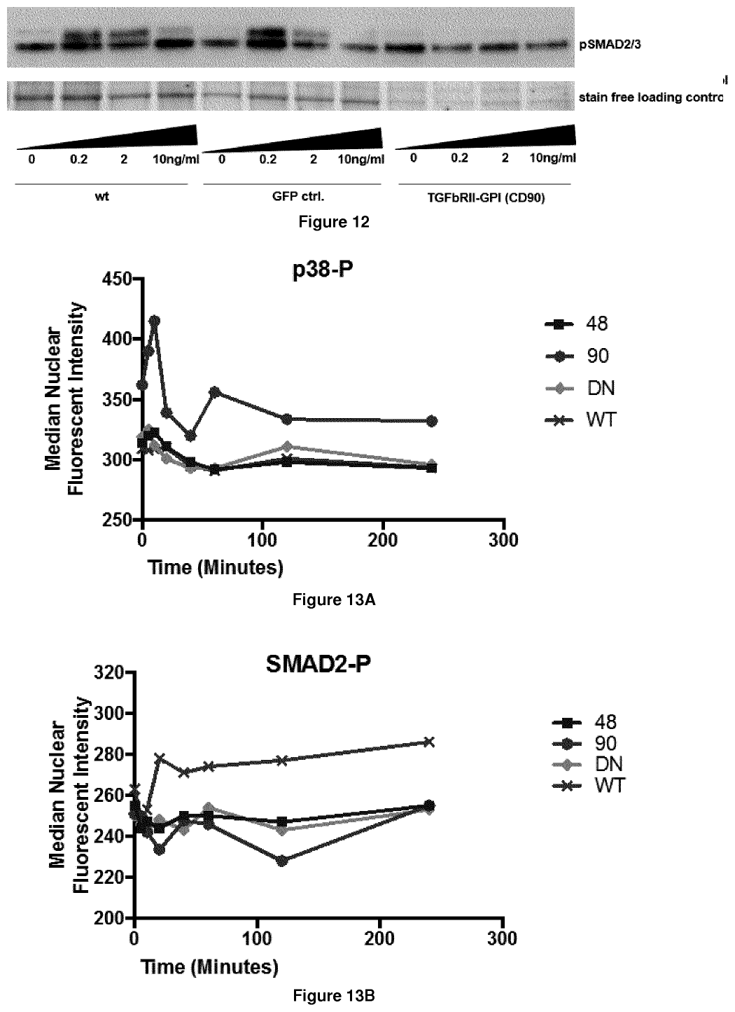

FIG. 12 shows phosphorylated SMAD2/3 detected in HeLa cells transduced with lentivirus encoding TGF-βRII-CD90 GPI anchor, lentivirus encoding GFP, or non-transduced cells, following stimulation with TGF-β.

Reduced levels of phosphorylated SMAD2/3 were detected in cells transduced with lentivirus encoding TGF-βRII-CD90 GPI anchor.

### 8.3 Fluorescence Microscopy

HeLa cells were transduced with lentivirus encoding TGF-βRII-CD90 GPI anchor, TGF-βRII-CD48 GPI anchor, DN-TGF-βR2 (see Example 6.2), or left untransduced (WT). The cells were then plated in wells of a 384 well plate and stimulated with 1 ng of TGF-β in a reverse time course experiment.

Phosphorylation and subcellular localisation of p38 and SMAD2 was analysed by fluorescent microscopy. Phosphorylation of p38 has been shown to be decreased in leukocytes stimulated with TGF-β, resulting in a decrease in immune function. SMAD2 phosphorylation has been shown to be a direct effector of TGF-β signalling and is responsible for decreased immune function.

The results of the experiment are shown in FIGS. 13A and 13B. Cells transduced with lentivirus encoding TGF-βRII-CD90 GPI anchor were found to display the highest level of phosphorylated p38 localised to the nucleus, and the lowest level of phosphorylated SMAD2 localised to the nucleus, in response to stimulation with TGF-β. This indicated that cells expressing TGF-βRII-CD90 decoy receptor were least responsive to TGF-β stimulation.

Cells transduced with lentivirus encoding TGF-βRII-CD48 GPI anchor displayed a similar level of phosphorylated p38 and phosphorylated SMAD2 localised to the nucleus following stimulation with TGF-β as compared to cells transduced with lentivirus encoding DN-TGF-βR2.

1. A TGF-β decoy receptor comprising a TGF-β binding region and a lipid anchor region.

2. The TGF-β decoy receptor according to claim 1, wherein the lipid anchor region comprises or consists of a lipid anchor.

3. The TGF-β decoy receptor according to claim 2, wherein the lipid anchor is a GPI anchor.

4. The TGF-β decoy receptor according to any one of claims 1 to 3, which lacks a transmembrane domain.

5. The TGF-β decoy receptor according to claim 1, wherein the lipid anchor region comprises or consists of an amino acid sequence which is a lipid anchor signal sequence.

6. The TGF-β decoy receptor according to claim 5, wherein the lipid anchor signal sequence is a glycosylphosphatidylinositol (GPI) signal sequence.

7. The TGF-β decoy receptor according to any one of claims 1 to 6, wherein the TGF-β binding region comprises or consists of an amino acid sequence corresponding to the TGF-β binding region of a TGF-β receptor.

8. The TGF-β decoy receptor according to claim 7, wherein the TGF-β receptor is the type II TGF-β receptor (TGF-βR2).

9. The TGF-β decoy receptor according to any one of claims 1 to 8, wherein the TGF-β binding region comprises or consists of an amino acid sequence having at least 80% amino acid sequence identity to SEQ ID NO:4.

10. The TGF-β decoy receptor according to any one of claims 1 to 9, wherein the cell membrane anchor region comprises or consists of an amino acid sequence corresponding to the GPI signal sequence of one of CD48, CD55, CD90 or placental-type alkaline phosphatase.

11. The TGF-β decoy receptor according to any one of claims 1 to 10, wherein the lipid anchor region comprises or consists of:
(i) an amino acid sequence having at least 80% amino acid sequence identity to any one of SEQ ID NOs:15 to 18; or
(ii) a GPI anchor, and an amino acid sequence having at least 80% amino acid sequence identity to any one of SEQ ID NOs:19 to 22.

12. The TGF-β decoy receptor according to any one of claims 1 to 11, comprising or consisting of:
(i) an amino acid sequence having at least 80% amino acid sequence identity to any one of SEQ ID NOs:5 to 8; or
(ii) an amino acid sequence having at least 80% sequence identity to one of SEQ ID NOs:11 to 14, and a GPI anchor.

13. A chimeric antigen receptor (CAR) comprising a TGF-β binding region which comprises or consists of an amino acid sequence corresponding to the TGF-β binding region of a TGF-β receptor.

14. The CAR according to claim 13, wherein the TGF-β receptor is the type II TGF-β receptor (TGF-βR2).

15. The CAR according to claim 13 or claim 14, wherein the TGF-β binding region comprises or consists of an amino acid sequence having at least 80% amino acid sequence identity to SEQ ID NO:4.

16. A nucleic acid encoding the TGF-β decoy receptor or CAR according to any one of claims 1 to 15.

17. A vector comprising the nucleic acid of claim 16.

18. A cell comprising the TGF-β decoy receptor or CAR according to any one of claims 1 to 15, the nucleic acid according to claim 16, or the vector according to claim 17.

19. A method for producing a cell expressing a TGF-β decoy receptor or CAR, comprising introducing into a cell a nucleic acid according to claim 16 or a vector according to claim 17, and culturing the cell under conditions suitable for expression of the nucleic acid or vector by the cell.

20. A cell which is obtained or obtainable by the method according to claim 19.

21. A pharmaceutical composition comprising the TGF-β decoy receptor or CAR according to any one of claims 1 to 15, the nucleic acid according to claim 16, the vector according to claim 17, or the cell according to claim 18 or claim 20, and a pharmaceutically acceptable carrier, adjuvant, excipient, or diluent.

22. The TGF-β decoy receptor or CAR according to any one of claims 1 to 15, the nucleic acid according to claim 16, the vector according to claim 17, the cell according to claim 18 or claim 20 or the pharmaceutical composition according to claim 21, for use in a method of treating or preventing a disease or condition.

23. Use of the TGF-β decoy receptor or CAR according to any one of claims 1 to 15, the nucleic acid according to claim 16, the vector according to claim 17, the cell according to claim 18 or claim 20 or the pharmaceutical composition according to claim 21, in the manufacture of a medicament for treating or preventing a disease or condition.

24. A method of treating or preventing a disease or condition, comprising administering to a subject a therapeutically or prophylactically effective amount of the TGF-β decoy receptor or CAR according to any one of claims 1 to 15, the nucleic acid according to claim 16, the vector according to claim 17, the cell according to claim 18 or claim 20 or the pharmaceutical composition according to claim 21.

25. A method of treating or preventing a disease or condition in a subject, comprising:
(a) isolating at least one cell from a subject;
(b) modifying the at least one cell to express or comprise the TGF-β decoy receptor or CAR according to any one of claims 1 to 15, the nucleic acid according to claim 16, or the vector according to claim 17, and;
(c) administering the modified at least one cell to a subject.

26. A method of treating or preventing a disease or condition in a subject, comprising:
(a) isolating at least one cell from a subject;
(b) introducing into the at least one cell the nucleic acid according to claim 16 or the vector according to claim 17, thereby modifying the at least one cell and;
(c) administering the modified at least one cell to a subject.

27. The TGF-β decoy receptor, CAR, nucleic acid, vector, cell, or pharmaceutical composition for use according to claim 22, the use according to claim 23, or the method according to any one of claims 24 to 26, wherein the disease or condition is a cancer.

28. A kit of parts comprising a predetermined quantity of the TGF-β decoy receptor or CAR according to any one of claims 1 to 15, the nucleic acid according to claim 16, the vector according to claim 17, the cell according to claim 18 or claim 20 or the pharmaceutical composition according to claim 21.


SEQUENCE LISTING (23 sequences)

SEQ ID NO: 1 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 2 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 3 - protein, 851 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 4 - protein, 104 aa, Homo sapiens
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 5 - protein, 218 aa, Artificial Sequence
  TGFbetaR2 ECD-CD48 GPI
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVCSPPCTLARSFGVEWIASWLVVTVPTILGLLLT

SEQ ID NO: 6 - protein, 224 aa, Artificial Sequence
  TGFbeta RS ECD-CD55 GPI
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPHETTPNKGSGTTSGTTRLLSGHTCFTLTGLLGTLVTMGLLT

SEQ ID NO: 7 - protein, 225 aa, Artificial Sequence
  TGFbetaR2 ECD-PLAC ALKPHOS GPI
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPTACDLAPPAGTTDAAHPGPSVVPALLPLLAGTLLLLGTATAP

SEQ ID NO: 8 - protein, 226 aa, Artificial Sequence
  TGFbetaR2 ECD-CD90 GPI
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPNVTVLRDKLVKCEGISLLAQNTSWLLLLLLSLSLLQATDFMSL

SEQ ID NO: 9 - protein, 183 aa, Artificial Sequence
  TGF-betaR2-derived region compirsing the TGF-beta binding region
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNP

SEQ ID NO: 10 - protein, 161 aa, Artificial Sequence
  TGF-betaR2 mature sequence
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNP

SEQ ID NO: 11 - protein, 173 aa, Artificial Sequence
  TGFbetaR2 ECD CD48 GPI
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVCSPPCTLARS

SEQ ID NO: 12 - protein, 174 aa, Artificial Sequence
  TGFbetaR2 ECD-CD55 GPI
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPHETTPNKGSGTTS

SEQ ID NO: 13 - protein, 174 aa, Artificial Sequence
  TGFbetaR2 ECD-PLAC ALKPHOS GPI
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPTACDLAPPAGTTD

SEQ ID NO: 14 - protein, 178 aa, Artificial Sequence
  TGFbetaR2 ECCD-CD90 GPI
TRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPNVTVLRDKLVKC

SEQ ID NO: 15 - protein, 35 aa, Artificial Sequence
  CD48 GPI
DVCSPPCTLARSFGVEWIASWLVVTVPTILGLLLT

SEQ ID NO: 16 - protein, 41 aa, Artificial Sequence
  CD55 GPI
HETTPNKGSGTTSGTTRLLSGHTCFTLTGLLGTLVTMGLLT

SEQ ID NO: 17 - protein, 42 aa, Artificial Sequence
  PLAC ALKPHOS GPI
TACDLAPPAGTTDAAHPGPSVVPALLPLLAGTLLLLGTATAP

SEQ ID NO: 18 - protein, 43 aa, Artificial Sequence
  CD90 GPI
NVTVLRDKLVKCEGISLLAQNTSWLLLLLLSLSLLQATDFMSL

SEQ ID NO: 19 - protein, 12 aa, Artificial Sequence
  CD48 GPI
DVCSPPCTLARS

SEQ ID NO: 20 - protein, 13 aa, Artificial Sequence
  CD55 GPI
HETTPNKGSGTTS

SEQ ID NO: 21 - protein, 13 aa, Artificial Sequence
  PLAC ALKPHOS GPI
TACDLAPPAGTTD

SEQ ID NO: 22 - protein, 12 aa, Artificial Sequence
  CD90 GPI
NVTVLRDKLVKC

SEQ ID NO: 23 - protein, 22 aa, Artificial Sequence
  TGF-betaR2 leader sequence
MGRGLLRGLWPLHIVLWTRIAS